(19) United States
(12) Patent Application Publication
(10) Pub. No.: US 2006/0123490 A1
(43) Pub. Date: Jun. 8, 2006

(54) Pluripotent cells with improved efficiency of homologous recombination and use of the same

(75) Inventors: Makoto Kakitani, Gunma (JP); Kazuma Tomizuka, Gunma (JP)

Correspondence Address: FOLEY AND LARDNER LLP;SUITE 500, 3000 K STREET NW, WASHINGTON, DC, 20007, US

(73) Assignee: Kirin Beer Kabushiki Kaisha

(21) Appl. No.: 11/212,101

(22) Filed: Aug. 26, 2005

(30) Foreign Application Priority Data
Aug. 30, 2004 (JP) 2004-250756
May 2, 2005 (JP) 2005-134380

(51) Int. Cl. A01K 67/00; C12N 5/06

(52) U.S. Cl. 800/8; 435/325

(57) ABSTRACT

The present invention provides a method of enhancing an efficiency of homologous recombination when a gene encoding a desired protein known or unknown in terms of function is introduced into a genome of a pluripotent cell such as ES cell. More particularly, the present invention relates to: a non-human animal-derived pluripotent cell comprising a foreign enhancer at a site downstream of an immunoglobulin gene on chromosome; a non-human animal pluripotent cell comprising a gene, which encodes a desired protein at a site downstream of the immunoglobulin gene and upstream of the foreign enhancer on the chromosome, said gene being in an overexpressible state; a method of establishing said pluripotent cell; and a chimeric non-human animal and its progeny produced by use of the pluripotent cells and a method of producing the same. The present invention further relates to a method of analyzing the function of a desired protein or a gene encoding the protein by comparing a phenotype of the chimeric non-human animal or its progeny with that of a control animal, and/or to a method of producing a useful protein by use of the chimeric non-human animal or its progeny.

### BACKGROUND OF THE INVENTION

1. Field of the Invention

The present invention relates to a pluripotent cell, such as ES cell, with improved efficiency of homologous recombination in a certain chromosomal region, to a method of establishing the pluripotent cell, and to a chimeric non-human animal produced using the pluripotent cell, and a progeny thereof.

The present invention also relates to a method of analyzing the function of a desired protein or a gene encoding the protein, and/or a method of producing a useful substance by use of the chimeric non-human animal and progeny thereof.

2. Background Art

Historical research outcomes of sequencing the entire human genome nucleotides (International Human Genome Sequencing Consortium, Nature, 409:860-921, 2001) have brought a new research subject of elucidating functions of a great number of novel genes. For example, in human chromosome 22, which is the second smallest of the 24 human chromosomes and whose entire nucleotide sequence was first determined (Dunham et al., Nature, 402:489-495, 1999), it was predicted that 545 genes (excluding pseudogenes) are present. Of them, 247 genes are known in terms of their nucleotide and amino acid sequences, 150 genes are novel ones that are homologous to known genes, and 148 genes are novel ones that are homologous to the sequences whose functions are unknown and which have been registered in the Expressed Sequence Tag (EST) database. In addition, through the analysis using the software (GENESCAN) which enables a direct prediction of a gene from the genomic sequences, it was predicted that there might exist further 325 novel genes whose transcriptional products have not been identified (Dunham et al., ibid.). Clarifying the in vivo functions of genes and proteins (as gene products) is important not only for understanding of a program of the life activity but also for development of a novel medicament to overcome a variety of human diseases. Thus, there is a big demand for development of techniques to efficiently elucidate the function of a novel gene in the post-genomic life science and medical researches.

The embryonic stem cell (or ES cell) refers to an undifferentiated cell line, which is established from an inner cell mass of the blastocyst and has an ability to differentiate into various types of somatic tissues including germ cells. In the case of mice, for example, when ES cell is injected into an early murine embryo (i.e., host embryo), a chimeric mouse is born having somatic cells which are a mixture of cells derived from the ES cell and the host embryo. In particular, a chimeric mouse having a germ cell derived from ES cell and capable of transmitting the genetic information of the ES cell to its progeny is called a germ-line chimera. When germ-line chimeras are mutually crossed, or when a germ-line chimera is crossed with an appropriate mouse line, F1 mice having the ES cell-derived genetic information is born. If ES cells are previously engineered in such techniques by modifying a certain gene in the cell or by inserting a certain gene into the cell, a knock-out (KO) mouse, transgenic (Tg) mouse, or knock-in (KI) mouse can be produced. From the analized outcomes of KO mice which have been so far produced by many researchers, important information and many human-disease animal models were provided or produced in a wide variety of fields from fundamental biology to clinical medicine. The KO mouse is still the most widely used tool for clarifying an in vivo biological function of a gene. On the other hand, the KI mouse is produced by inserting a certain foreign gene into a particular murine gene in the manner of homologous recombination (Le Mouellic et al., 2002; Japanese Patent No. 3,298,842) or of random insertion (Gossler et al., Science, 244: 463-465, 1989). Furthermore, mice produced by inserting an expression unit comprising a certain promoter, a foreign gene, and a poly A addition site, into a particular chromosomal region have been reported as suitable for analyzing the in vivo functions of many genes (Tomizuka et al., PCT International Application No. WO 03/041,495).

However, for the Tg mouse, KI mouse or KO mouse, a lot of time and labor are required for manipulating only a single gene. Usually, the efficiency of homologous recombination is about one per 100-10,000 random insertion clones. To improve the ratio of homologous recombinants to randomly inserted recombinants, various attempts have been hitherto made. For example, Deng & Capecchi (Mol. Cell. Biol., 12:3365-71, 1992) reported that the longer the length of a genomic DNA of the homologous region contained in a vector, the more preferable, and that it is preferable to use the isogenic DNA which is a genomic DNA from the same murine species as that from which ES cell for use in targeting is derived. Furthermore, the method most widely used at present is to employ KO vectors comprising a negative selection marker outside the homologous genomic DNA region, in addition to the said selection marker. The negative selection method utilizes a phenomenon where the cells having random inserts die because of expression of a virulent negative marker, whereas the homologous recombinants survive because such a virulent expression does not occur. Examples of the negative selection marker include HSV-tk gene (in this case, culture medium must contain a thymidine analogue such as ganciclovir or FIAU) reported by Mansour et al. (Nature, 336:348-352, 1988), and DT-A (diphtheria toxin A chain) reported by Yagi et al. (Anal. Biochem., 214:77-86, 1993). When the negative selection theoretically works, all colonies presumably become homologous recombinants. However, actually, the rate of homologous recombinants greatly varies in from report to report. The efficiency of obtaining homologous recombinants by the negative selection method (i.e., the concentration effect) is generally several folds higher than other methods.

Not only mice produced from mutant ES cell lines but also mammalian cell lines, which have a gene modified or destroyed by homologous recombination, are important materials in clarifying a function of the modified or destroyed gene. Furthermore, homologous recombination has been considered as an ultimate therapy for diseases (especially, hereditary diseases) caused by defect or mutation of a gene. Nevertheless, the ratio of homologous recombinants to randomly inserted recombinants in the mammalian cell lines or the primary culture cells is equal to or lower than in murine ES cells. In this context, it has been desired to improve said ratio in applying this method to gene-function analysis and gene therapy at a cellular level (Yanez & Poter, Gene Therapy, 5:149-159, 1998).

In the present stage where the entire human genome nucleotide sequence has been determined, what is desired next is a system capable of exhaustively analyzing in vivo functions for multiple novel genes. For this purpose, it is necessary to reliably, easily and simultaneously produce a plurality of types of animal individuals capable of highly expressing a transfer gene. Of the novel genes brought by the human genomic information, genes encoding secretory proteins homologous to cytokines, growth factors, and hormones are interested as research subjects since they directly act as medicaments. In other words, developing new efficient methods of analyzing in vivo functions of genes encoding secretory proteins or gene products presumably facilitates development of medicaments for treating human diseases.

### DISCLOSURE OF THE INVENTION

An object of the present invention is to provide a pluripotent cell, such as ES cell, with improved efficiency of homologous recombination in a particular chromosomal region. Another object of the present invention is to provide a chimeric non-human animal or progeny thereof prepared by using the pluripotent cells genetically modified so as to overexpress a desired gene. A further object of the present invention is to provide a simple and highly reproducible method of analyzing the function of a target gene or protein therefor and/or producing a useful protein by use of the chimeric non-human animal or its progeny.

We conducted intensive studies in order to achieve the aforementioned objects, and as a result, have now found that the ratio of homologous recombinants to randomly inserted recombinants (or unhomologous recombinats) in a gene targeting vector, which contains a gene encoding an exogenous or endogenous protein with known or unknown function at a site downstream of an immunoglobulin gene and upstream of a foreign enhancer, can be greatly improved by use of pluripotent cells (such as ES cells) having the foreign enhancer inserted into a particular chromosomal region, i.e., a site downstream of the immunoglobulin gene. Based on the findings, the present invention was successfully achieved. It was not known that modification previously applied to a particular region on the chromosome had an effect on homologous recombination efficiency in the gene targeting using a targeting vector which contained no sequence of the same region. Hence, it was a surprising finding.

Furthermore, we succeeded in producing a chimeric non-human animal (e.g., mouse) by injecting a pluripotent cell, such as ES cell, genetically modified into a B cell-deficient host embryo. In the chimeric non-human animal or progeny thereof produced in accordance with the method of the present invention, overexpression of a product derived from the introduced structural gene was observed irrelevant to the chimeric rate of hair-color. It was thus confirmed that chimeric non-human animals or progeny thereof capable of highly expressing a transfer gene could be obtained efficiently by use of this system without failure compared to conventional methods.

### SUMMARY OF THE INVENTION

The present invention will be summarized as follows.

(1) A pluripotent cell derived from a non-human animal, comprising a foreign enhancer at a site downstream of an immunoglobulin gene on chromosome.

(2) The cell of (1) above, wherein the foreign enhancer is located at a site within 100 Kb or less, preferably 50 Kb or less, and more preferably 30 Kb or less, downstream of the 3′ end of the immunoglobulin gene.

(3) The cell of (2) above, wherein the foreign enhancer is located at a site within 30 Kb or less downstream of the 3′ end of the immunoglobulin gene.

(4) The cell of (3) above, wherein the foreign enhancer is located at a site of RS element or in the vicinity of the RS element.

(5) The cell of any of (1) to (4) above, wherein the foreign enhancer is derived from a virus.

(6) The cell of (5) above, wherein the virus is an infectious mammalian virus.

(7) The cell of item (6), wherein the infections mammalian virus is SV40.

(8) The cell of any of (1) to (7) above, wherein the immunoglobulin gene is a gene for the variable or constant region of the heavy chain or light chain of the immunoglobulin.

(9) The cell of (8) above, wherein the immunoglobulin gene is a gene for the constant region of the heavy chain or light chain of the immunoglobulin.

(10) The cell of (9) above, wherein the immunoglobulin gene is a κ light-chain constant region gene.

(11) The cell of any of (1) to (10) above, wherein the non-human animal is a mammal.

(12) The cell of (11) above, wherein the mammal is a rodent.

(13) The cell of (12) above, wherein the rodent is a mouse.

(14) The cell of any of (1) to (13) above, wherein the pluripotent cell is an embryonic stem (ES) cell.

(15) The cell of (14) above, wherein the ES cell is from a mouse.

(16) The cell of any of (1) to (15) above, wherein the foreign enhancer is contained together with a first foreign gene (referred to as a “first gene”) under the control of the foreign enhancer.

(17) The cell of (16) above, wherein the first gene is a drug resistant gene.

(18) The cell of (17) above, wherein the drug resistant gene is a neomycin resistant gene.

(19) A method of producing a pluripotent cell derived from a non-human animal of any of (1) to (18) above, comprising:

preparing a gene targeting vector comprising a sequence homologous to a 5′ region upstream of a foreign enhancer-inserting position on a chromosome of the pluripotent cell, a sequence comprising the foreign enhancer, and a sequence homologous to a 3′ region downstream of the foreign enhancer-inserting position; and

introducing the gene targeting vector into a pluripotent cell derived from a non-human animal, thereby integrating the foreign enhancer at a site downstream of an immunoglobulin gene,

wherein the position into which the foreign enhancer has been inserted is a site downstream of the immunoglobulin gene, preferably a site within 100 Kb or less, more preferably 50 Kb or less, and far more preferably 30 Kb or less downstream of the 3′ end of the immunoglobulin gene.

(20) The method of (19) above, wherein the vector further comprises a first gene under the control of the foreign enhancer.

(21) The method of (20) above, wherein the first gene is a drug resistant gene.

(22) The method of (21) above, wherein the drug resistant gene is a neomycin resistant gene.

(23) The method of any of (19) to (22) above, wherein the foreign enhancer is derived from a virus.

(24) The method of (23) above, wherein the virus is an infectious mammalian virus.

(25) The method of (24) above, wherein the infectious mammalian virus is SV40.

(26) The method of any of (19) to (25) above, wherein the non-human animal is a mammal.

(27) The method of (26) above, wherein the mammal is a rodent.

(28) The method of (27) above, wherein the rodent is a mouse.

(29) The method of any of (19) to (28) above, wherein the insertion position of the foreign enhancer falls within 30 Kb or less from the 3′ end of the immunoglobulin gene.

(30) The method of (29) above, wherein the inserted position of the foreign enhancer is a site of RS element or in the vicinity of the RS element.

(31) The method of any of (19) to (30) above, wherein the gene targeting vector has a structure shown in FIG. 7 and the sequence comprising the foreign enhancer has a structure shown in FIG. 8.

(32) The method of any of (19) to (31) above, wherein the pluripotent cell is an ES cell.

(33) The method of (32) above, wherein the ES cells are from a mouse.

(34) A method of introducing a desired second foreign gene (referred to as a “second gene”) (whose function is known or unknown) into a chromosome of a pluripotent cell derived from a non-human animal, comprising introducing the second gene expressably by means of homologous recombination into a site downstream of an immunoglobulin gene on chromosome of the pluripotent cell and upstream of the foreign enhancer in the pluripotent cell of any of (1) to (18) above.

(35) The method of (34) above, wherein the second gene is introduced by use of a gene targeting vector comprising it.

(36) The method of (35) above, wherein the vector further comprises a promoter for controlling expression of the second gene.

(37) The method of (36) above, wherein the promoter is an immunoglobulin gene promoter.

(38) The method of any of (35) to (37) above, wherein the vector further comprises a multicloning site, poly A signal sequence, and positive and negative selection marker sequences.

(39) The method of any of (34) to (38) above, wherein the immunoglobulin gene is a light-chain constant region gene.

(40) The method of (39) above, wherein the immunoglobulin gene is a κ light-chain constant region gene.

(41) The method of any of (34) to (40) above, wherein the vector has the second gene inserted into the multicloning site in the structure shown in FIG. 3.

(42) The method of any of (34) to (41) above, wherein the non-human animal is a mammal.

(43) The method of (42) above, wherein the mammal is a rodent.

(44) The method of (43) above, wherein the rodent is a mouse.

(45) The method of any of (34) to (44) above, wherein the pluripotent cell is an ES cell.

(46) The method of (45) above, wherein the ES cell is from a mouse.

(47) A cell derived from the non-human animal-derived pluripotent cell of any of (1) to (18) above, wherein a second gene (whose function is known or unknown) is further comprised at a site downstream of the immunoglobulin gene on chromosome and upstream of the foreign enhancer in the pluripotent cell.

(48) The cell of (47) above, wherein the non-human animal is a mammal.

(49) The cell of (48) above, wherein the mammal is a rodent.

(50) The cell of (49) above, wherein the rodent is a mouse.

(51) The cell of any of (47) to (50) above, wherein the pluripotent cell is an ES cell.

(52) The cell of (51) above, wherein the ES cell is from a mouse.

(53) The cell of any of (47) to (52) above, wherein the foreign enhancer is derived from a virus.

(54) The cell of (53) above, wherein the virus is infectious mammalian virus.

(55) The cell of (54) above, wherein the infectious mammalian virus is SV40.

(56) The cell of any of (47) to (55) above, wherein the immunoglobulin gene is a light-chain gene.

(57) The cell of (56) above, wherein the immunoglobulin gene is a light-chain constant region gene.

(58) The cell of (57) above, wherein the immunoglobulin gene is a κ light-chain constant region gene, for example a murine κ light-chain constant region gene.

(59) The cell of any of (47) to (58) above, wherein the foreign enhancer is located at a site within 100 Kb or less, preferably 50 Kb or less, and more preferably 30 Kb or less downstream of the 3′ end of the immunoglobulin gene.

(60) The cell of (59) above, wherein the foreign enhancer is located at a site within 30 Kb or less downstream of the 3′ end of the immunoglobulin gene.

(61) The cell of (60) above, wherein the foreign enhancer is located at a site of RS element or in the vicinity of the RS element.

(62) A method of producing a chimeric non-human animal in which a second gene is overexpressed, comprising injecting a pluripotent cell derived from a non-human animal of any of (47) to (61) above into a host embryo, transplanting the obtained host embryo into a surrogate mother of the same species of non-human animal, and permitting the surrogate mother to give birth, thereby producing the chimeric non-human animal.

(63) The method of (62) above, comprising injecting a pluripotent cell into the blastocyst or 8-cell embryo from a non-human animal host in which a particular cell and/or tissue is in defect (for example, B cell-defective host embryo), transplanting the blastocyst or 8-cell embryo into the surrogate mother of a nonhuman animal, and permitting the surrogate mother to give birth, thereby producing a chimeric non-human animal.

(64) The method of (62) or (63) above, wherein the chimeric non-human animal is a mouse.

(65) A chimeric non-human animal with a second gene overexpressed, the animal being produced by the method according to any of (62) to (64) above or by injecting a pluripotent cell from the non-human animal of any of (47) to (61) above into a non-human animal host embryo.

(66) The chimeric non-human animal of (65) above, wherein the animal is a mouse.

(67) A non-human animal progeny with a desired foreign gene overexpressed, the progeny being produced by crossing chimeric non-human animals of (65) or (66) above with each other.

(68) The progeny of the non-human animal of (67) above, wherein the animal is a mouse.

(69) A method of analyzing a function of a desired foreign gene, comprising comparing a phenotype based on a second gene (i.e., a desired foreign gene) which is overexpressed in a chimeric non-human animal of claim (65) or (66) or a non-human animal progeny of claim (67) or (68), with that of a control animal, and analyzing the function of the gene based on difference in phenotype.

(70) The method of (69) above, wherein the animal is a mouse and the pluripotent cell is an ES cell.

(71) A method of producing a useful protein by expressing a second gene in a chimeric non-human animal of claim (65) or (66) or a non-human animal progeny of claim (67) or (68), and recovering a produced protein, which is encoded by the gene expressed.

(72) The method of (71) above, wherein the animal is a mouse.

(73) The method of (71) or (72) above, comprising producing the useful protein by use of any one of a tissue or cell of the animal or a hybridoma with B cell or spleen cell; and recovering the protein.

(74) The method of (73) above, wherein the tissue or cell is a lymphatic tissue or a B cell.

(75) The method of (73) above, wherein the hybridoma is a fusion cell of B cell or spleen cell with a proliferable tumor cell.

According to the present invention, specific embodiments are as follows.

The present invention provides an ES cell in which the efficiency of homologous recombination has been improved in the vicinity of a certain chromosomal region by inserting a drug-resistant marker gene expression unit comprising a foreign enhancer into at least one allele of the chromosomal region. As the chromosomal region into which the drug resistant maker is to be inserted, a genetic sequence called an RS sequence, particularly an RS sequence present downstream of the immunoglobulin κ-light-chain gene, is preferable.

The present invention also provides a genetic recombinant non-human animal or a chimeric non-human animal produced by use of the ES cell in which the efficiency of homologous recombination has been improved in the vicinity of a certain chromosomal region by inserting a drug-resistant marker gene expression unit comprising a foreign enhancer into at least one allele of the chromosomal region.

The present invention further provides a method of analyzing the function of a certain gene or a protein encoded by the gene, comprising producing a chimeric non-human animal expressing a certain gene by use of the ES cell in which the efficiency of homologous recombination has been improved in the vicinity of a certain chromosomal region by inserting a drug-resistant marker gene expression unit comprising a foreign enhancer into at least one allele of the chromosomal region, and comparing a phenotype of the chimeric non-human animal with that of a control animal.

In the present invention, the chimeric non-human animal is selected from the group consisting of mouse, cow, pig, monkey, rat, sheep, goat, rabbit and hamster. According to a preferable embodiment of the present invention, the chimeric non-human animal is a mouse.

Definition

The terms pertinent to the present invention are defined as follows.

The “foreign enhancer” as used herein is an exogenous or endogenous enhancer artificially introduced. The enhancer refers to a control region serving as the site to which a regulatory protein for activating transcription of a gene specifically binds. According to the invention, the enhancer was identified as a cis-acting DNA nucleotide sequence capable of increasing the level of transcription without depending on the orientation to or the distance from an RNA-transcriptional initiation site. The enhancer is known to be present in the vicinity of a promoter of a gene or sometimes within an intron so as to act there, or also to act at a distal distance from the enhancer. For example, the enhancer present in a “cut” locus of a drosophila is known to locate 85 kb upstream of a promoter, and the enhancer for T cell receptor α-chain gene is known to locate 69 kb downstream of the promoter (Blackwood et al., Science, Vol. 281, 60-63, 1998). Furthermore, the locus control region (LCR), which was identified as a DNA nucleotide sequence capable of highly expressing a transgene inserted in the genome in a position-independent manner, is known to contain a sequence functioning as an enhancer (Blackwood et al., ibid)

The term “foreign” as used herein refers to artificially introducing a substance such as nucleic acid externally irrespective of whether the substance is exogenous or endogenous, or refers to the substance thus introduced.

The term “non-human animal” as used herein refers to an animal excluding a human and is generally selected from vertebrates including fish, reptile, amphibian, bird, and mammal, preferably mammals. Since chimeric non-human animals are preferably produced by use of embryonic stem cells as pluripotent cells in the invention, any non-human animals from which embryonic stem cells can be established (for example, mouse, cow, sheep, pig, hamster, monkey, goat, rabbit, and rat), or any other non-human animals from which embryonic stem cells will be established in future, are encompassed in the non-human animal to be intended by the invention.

The term “chimeric non-human animal” as used herein refers to an animal established from differentiated cells derived from a pluripotent cell (as described below) or a host embryo (Bradley et al., Nature, 309:255-6, 1984). Experimentally, animals whose cells are completely from a host embryo (0% chimera) or animals whose cells are completely from a pluripotent cell (100% chimera) could be born. Such animals are not strictly “chimera” but are included in the “chimeric non-human anima” for the convenience sake.

The term “pluripotent cell” as used herein refers to a cell capable of differentiating into at least two types of cells or tissues of a chimeric non-human animal which is produced by injecting the cell into a host embryo or by forming an aggregated embryo. Specific examples of the pluripotent cell include embryonic stem cells (ES cells), embryonic germ cells (EG cells) and embryoniccarcinoma cells (EC cells).

The term “embryonic stem cell” as used herein, also called ES cell, refers to a cultured cell derived from the early embryo and characterized in that it has a proliferative potency while maintaining an undifferentiated state (or totipotency). In other words, the embryonic stem cell means a cell line established by culturing a cell of inner cell mass, i.e. an undifferentiated stem cell present in the early embryo (blastocyst stage) of an animal, so that the cell line can continuously proliferate while keeping an undifferentiated state. The term “embryonic germ cell,” also called “EG cell,” refers to a cultured cell derived from the primordial germ cell and characterized in that it has almost the same potency as that of the embryonic stem cell. The embryonic germ cell means a cell line established by culturing the primordial germ cell obtained from the embryo of several days to several weeks after fertilization (for example, about 8.5-day old embryo in mouse), so that the cell line can continuously proliferate while keeping an undifferentiated state. The term “embryonic tumor cell” refers to a cell having the same differentiation potency as that of the ES cell and is known as a stem cell established from the primordial germ cell, which is destined to be differentiated into a germ cell in future, in the presence of leukocyte inhibitory factor (LIF) and/or basic fibroblast growth factor (bFGF). The EG cell contributes to formation of the germ cells from which progeny can be produced.

As described in Colin L. Stewart et al. (The EMBO Journal, 4(13B), 3701-3709 (1985)) for the EC cells, and in Patricia A. Labosky et al. (Development 120, 3197-3204 (1994)) for the EG cells, both the EC cell and EG cell have a chimera forming potency like ES cell. It is confirmed that a foreign gene is expressed in a chimeric mouse derived from EC cells, while the EG cells contribute to formation of the germ line cells and production of progeny. As described above, the ES cell, EC cell, and EG cell all are applicable and encompassed in the present invention.

The term “a desired protein” as used herein refers to a protein that is to be intentionally expressed in at least one type of cells and/or tissue of a chimeric non-human animal produced by the method of the present invention. It is no matter whether the protein is known or unknown in function. Examples of the desired proteins may be mammalian proteins such as functional secretory proteins, functional membrane proteins, functional intracellular or intranuclear proteins, and soluble portions of functional membrane proteins with added secretory signal. The term “functional” as used herein means to possessing a specific role, effect or function in vivo.

In the case of a protein known in function, a new finding as to interrelation between functions of the protein may be provided by observing what effect is brought by the protein when it is highly expressed in at least one type of cells and/or tissue of a chimeric non-human animal. In the case of a protein unknown in function, a hint for elucidating the function of the protein may be found by observing any effect brought by the protein when it is highly expressed. In the present invention, the “desired protein” is expressed in a chimeric non-human animal into which a gene encoding the protein is introduced; however, it may be acceptable if it is not expressed or slightly expressed in certain cells and/or tissue of interest wherein the protein is intended to be expressed. Also, the “desired protein” may be derived from a xenogenic animal. As long as it is a “desired protein” of interest, any types of proteins may be used.

The “nucleic acid sequence encoding a desired protein” as used herein may be either endogenous or exogenous DNA. Also exogenous DNA includes a DNA derived from human. In the specification, the terms “a (structural) gene encoding a desired protein,” “a foreign gene encoding a desired protein” and “a desired foreign gene” are interchangeably used.

The term “expression” of a protein as used herein has the same meaning as expression of a gene encoding the protein.

The term “control region” as used herein refers collectively to “control sequence,” “regulatory sequence” and “regulatory region” and indicates a region for controlling or regulating gene expression (i.e., transcription, translation, or protein synthesis). Examples of such a control region include, but not limited to, a promoter, enhancer, and silencer. Also the term “control region” as used herein may contain a functional element (such as a promoter sequence) or a plurality of elements (such as a promoter sequence and an enhancer sequence). Furthermore, the “promoter sequence” is a kind of control region known by those skilled in the art and indicates a nucleotide sequence upstream of a structural gene to which RNA polymerase is bound at the initiation time of translation.

The term “internal ribosomal entry site” as used herein is simply referred to as “IRES” and is known as an element enabling polycistronic expression. The IRES forms a specific RNA secondary structure and is a site enabling initiation of ribosomal translation from an initiation codon downstream thereof. In the case of a mammal, the IRES binds to a decode subunit of a ribosome thereby causing a conformational change such that a protein coding region adjacent to the decode subunit is pulled into the decoding site. In this manner, IRES is presumably involved in the event initiating the translation and protein synthesis (Spahn et al. Science 291:1959, 2001).

The term “poly A signal region” or “poly A signal sequence” as used herein refers to a nucleotide sequence, which is positioned at the end of the transcription region and directs polyadenylation to the 3′ non-translation region of pre-mRNA after transcription.

The terms “upstream” and “downstream” as used herein refer to the direction to 5′ end or 3′ end, respectively, in a nucleic acid sequence such as genome or polynucleotide.

The terms “bp (base pair)” and “Kb or kb (kilo base pair)” as used herein refer to the length or distance of a nucleic acid sequence. “One (1) bp” indicates a single base pair, and “1 Kb” corresponds to 1,000 bp.

The term “allele” as used herein refers to genes which are located in homologous regions of a homologous chromosome in an organism having a polyploidal genome and are functionally homologous. The allele is usually all expressed. The term “allelic exclusion” refers to the phenomenon where phenotypes derived from both allelic genes are expressed in an organism individual, however, one of the allelic genes is only expressed at random in individual cells, whereas expression of the other gene is excluded. This phenomenon is usually seen in antibody genes and T cell receptor genes, wherein because the recombination of one allelic variable region gene is interpreted as a signal, only one complete gene is produced.

The term “a soluble portion of a membrane protein with added secretory signal” as used herein refers an extracellular domain of membrane protein molecule to which a secretory signal (or signal sequence) is bound.

The term “immunoglobulin gene” as used herein refers to a gene encoding a light chain (or L-chain) or a heavy chain (or H-chain) of an immunoglobulin (Ig) molecule. The light chains include κ-chain and λ-chain, each of which consists of variable (V) and constant (C) regions. The light-chain gene is constituted of a single constant region gene, a plurality of V region genes, and a plurality of joint (J) region genes. On the other hand, in a mammal, there are several types of heavy-chain genes including μ, γ, α, δ, and ε (note that δ gene is present in a human, monkey or mouse but not in a rat, cow, horse or rabbit) and several types of constant region genes. Heavy-chain genes μ, γ, and α are present in birds; heavy-chain genes μ, and γ are present in reptiles and amphibians; and heavy-chain gene μ is only present in fish. Considering that usually a gene encoding a desired protein is inserted into a single site of a chromosome, it is preferable to use the κ light chain constant region gene derived from a mammal. Note that the heavy-chain gene of a mouse is present on chromosome 12, while the κ light-chain and λ light-chain genes are present on chromosome 6 or chromosome 16, respectively. Furthermore, the immunoglobulin gene has the V and C region determining genes, which are arranged in order from the 5′ side, further comprising diversity segment (D) and joining segment (J) region determining genes between the V and C region determining genes.

The term “a host embryo of a non-human animal devoid of certain cells and/or tissue” or “defective host embryo” refers to a host embryo of a non-human animal to which pluripotent cell is to be injected and which is devoid of the certain cells and/or tissue.

The term “progeny” of a chimeric non-human animal as used herein refers to a non-human animal, which is obtained by mutually crossing chimeric non-human animals according to the present invention or by crossing a chimeric non-human animal according to the present invention with a cognate non-human animal, and which is capable of expressing a desired protein at least in the certain cells and/or tissue.

The term “phenotype” as used herein refers to a trait inherent in an animal or a trait of an animal emerging as a result of gene introduction.

The term “proliferable tumor cell” as used herein refers to a tumorigenic cell having permanent proliferable potency, e.g., plasmacytoma (or myeloma cells) which can use to produce immunoglobulins.

The term “hybridoma” as used herein refers to a hybrid cell obtainable by fusing a cell derived from the tissue or cell of a chimeric non-human animal according to the present invention and its progeny, with a proliferable tumor cell.

The term “targeting vector” or “gene targeting vector” as used herein refers to a vector having an expression unit of a gene encoding a desired protein. When the vector is introduced into a target chromosome region by means of homologous recombination, the desired protein is expressed. The term “knock out vector” as used herein refers to a vector for use in destroying or inactivating a desired gene of a non-human animal by homologous recombination. Furthermore, the term “knock out” or “gene knock out” refers to destroying or inactivating a target gene by introducing a structure for inhibiting the expression of the gene into a target locus by homologous recombination.

The term “recombining segment (RS) element” as used herein refers to a sequence such as agtttctgca cgggcagtca gttagcagca ctcactgtg (SEQ ID NO:39), which is located about 25 Kb downstream of the immunoglobulin κ light chain constant region gene on the murine chromosome 6, and has nonamer and heptamer signal sequences (Daitch et al., J. Immunol., 149: 832-840, 1992). As a result of analysis, most of the B cells expressing λ chain are deficient in Cκ exon or Jκ-Cκ region. It is reported that this deficiency is due to recombination between said exon or region and a DNA sequence (i.e., recombining sequence or RS element) located 25 kb downstream of the Cκ exon on the murine chromosome 6 (Durdik et al., Nature, 307:749-752, 1984; Moore et al., Proc. Natl. Acad. Sci. USA, 82:6211-6215, 1985; and Muller et al., Eur. J. Immunol., 20:1409-1411, 1990). The RS element binds to a site positioned in the intron between Jκ and Cκ or binds to Vκ gene, whereby the recombination occurs. The recombination is conceivably mediated by the same enzyme as used in the V(D)J joining of Ig (Durdik et al., ibid; Moore et al., ibid). The role of the RS element in recombination for developing B cells is not sufficiently elucidated; however, the RS element is considered to play a role in suppressing transcription of κ gene, which has a sequence structure that is rendered nonfunctional by frame shift at least when the Ig gene is reconstituted), and also in suppressing the expression of κ chain even in the B cells expressing λ chain. It is also known that self-reactive B cell, which is generated during the B cell differentiation stage, stops light chain production and activates reconstitution (called receptor editing) of the light-chain gene (Radic et al., J. Exp. Med., 177:1165-1173, 1993; Tiegs et al., J. Exp. Med., 177:1009-1020, 1993). Alternatively, it is pointed out that the recombination via RS element may possibly be responsible for inactivation (called the receptor proofreading) of functional κ gene, (Selsing et al., IMMUNOGLOBULIN GENES, SECOND EDITION, ACADEMIC PRESS, 200-203, 1995).

The present invention relates to a method of introducing a gene encoding a desired protein in a homologous recombination manner by using the expression system of an immunoglobulin gene, particularly κ light-chain constant region gene, in pluripotent cells. The invention also relates to a pluripotent cell obtained by said method, and to a chimeric non-human animal and progeny thereof as produced from the pluripotent cell. The rate of recombination in the Igκ locus reaches 20% or more, 25% or more, 30% or more, 40% or more, 50% or more, preferably 60% or more. Thus, the present invention has remarkable advantages in that desired protein can be produced by highly expressing a gene encoding it, and in that the biological function of a gene or protein with unknown function can be elucidated.

The specification includes the contents as described in the specifications and/or drawings of Japanese Patent Application No. 2004-250,756 and No. 2005-134,380, whose priorities are claimed in the present application.

### BRIEF DESCRIPTION OF THE DRAWINGS

FIG. 1 shows the structure of a murine RS element targeting vector, pBlueRS-LoxP-Neo-DT-A-3′KO-5′KO, wherein 5′KO is the 5′ reagion upstream of the murine RS element, Neo^{r} is a neomycin resistant gene, 3′KO is the 3′ reagion downstream of the murine RS element, DT-A is a diphtheria toxin A chain gene, and pBluescript is a cloning vector.

FIG. 2 shows the allelic structure in which the neomycin resistant gene has been inserted in place of the murine RS element, and the positions of probes for Southern analysis, wherein 5′ genome is the 5′ region upstream of the murine RS element, 3′ genome is the 3′ region downstream of the murine RS element, 5′ probe is a probe for Southern analysis to confirm insertion of a targeting vector into the 5′ side, 3′ probe is a probe for Southern analysis to confirm insertion of a targeting vector into the 3′ side, and loxP-neo-loxP is a neomycin resistant gene.

FIG. 3 shows the structure of a CκP2 targeting vector, wherein Promoter 2 is murine Igκ promoter region gene 2, MCS is a multicloning site, Cκ is the murine Igκ gene constant region, Cκ polyA is a poly A signal region of the murine Igκ, Puro is a puromycin resistant gene, DT-A is a diphtheria toxin A chain gene, and pBluescript is a cloning vector.

FIG. 4 is the structure of a CκP2 targeting vector which has a human EPO gene inserted into the cloning site, wherein Promoter 2 is murine Igκ promoter region gene 2, hEPO is a human EPO gene, Cκ is the murine Igκ gene constant region, Cκ poly A is a poly A signal region of the murine Igκ, Puro is a puromycin resistant gene, DT-A is a diphtheria toxin A chain gene, and pBluescript is a cloning vector.

FIG. 5 shows the allelic structure in which human EPO gene was targeted and the position of a probe for Southern analysis, wherein P2 is murine Igκ promoter region gene 2, EPO is a human EPO gene, Cκ is the murine Igκ gene constant region, poly A is a poly A signal region of the murine Igκ, Puro is a puromycin resistant gene, DT-A is a diphtheria toxin A chain gene, and probe is a probe for Southern analysis.

FIG. 6 shows removal of a Neo unit by Cre recombinase.

FIG. 7 shows the structure of a modified murine RS element targeting vector, pRS-KOSV40PE, wherein 5′ genome is the 5′ region upstream of the murine RS element, 3′ genome is the 3′ region downstream of the murine RS element, DT-A is a diphtheria toxin A chain gene, and loxP-Neo-loxP is a neomycin resistant gene.

FIG. 8 shows a drug resistant marker gene expression unit comprising SV 40 enhancer/promoter (SV40PE), HSV-TK promoter, Neo resistant marker gene, SV40 poly A and LoxP, wherein HSV-TK is thymidine kinase from herpes simplex virus.

FIG. 9 shows the genomic structure of a region in the vicinity of the RS element in the removal step of the Neo resistant marker gene from the modified RS element targeting murine ES cell line targeted by the vector pRS-KOSV40PE.

FIG. 10 shows the wild-type genomic structure (WT) in the vicinity of the Igκ constant region of a murine ES cell; the genomic structure (ΔRS) having the drug resistant gene (neo^{r}) inserted in place of the RS element region (RS); and the targeting vector for introducing a desired gene into a region in the vicinity of the Igκ constant region by homologous recombination.

FIG. 11 shows the structure of a vector, pRS-KOSV4072bp, wherein 5′ genome is the 5′ region upstream of the murine RS element, 3′ genome is the 3′ region downstream of the murine RS element, DT-A is a diphtheria toxin A chain gene, and loxP-Neo-loxP is a neomycin resistant gene.

FIG. 12 shows removal of the Neo resistant marker gene from the murine ES cell line targeted by the vector pRS-KOSV4072bp.

### DETAILED DESCRIPTION OF THE PREFERRED EMBODIMENTS

The present invention will be described in more detail below.

The present invention provides a pluripotent cell derived from a non-human animal, characterized in that the pluripotent cell comprises a foreign enhancer at a site downstream of an immunoglobulin gene on the chromosome.

The foreign enhancer is, as defined above, a control sequence serving as the site to which a gene regulatory protein for activating transcription specifically binds. The enhancer generally has an effect of increasing a transcriptional initiation rate. In the present invention, it is presumed that, when a DNA binding protein binds to the enhancer, the structure of the chromatin changes to some extent, thereby improving the efficiency of homologous recombination of a gene encoding a desired protein or a desired gene on the chromosome. Examples of such a foreign enhancer include, but not limited to, enhancers for viral genes, for example enhancers for infectious mammalian virus genes, such as SV (simian virus) 40 gene, polyoma virus gene, bovine papilloma virus gene, adenovirus E1A gene, retrovirus gene, and cytomegalovirus gene; and enhancers for nuclear genes of cells, such as immunoglobulin gene, chymotrypsin gene, and insulin gene. Of them, viral enhancers are preferred.

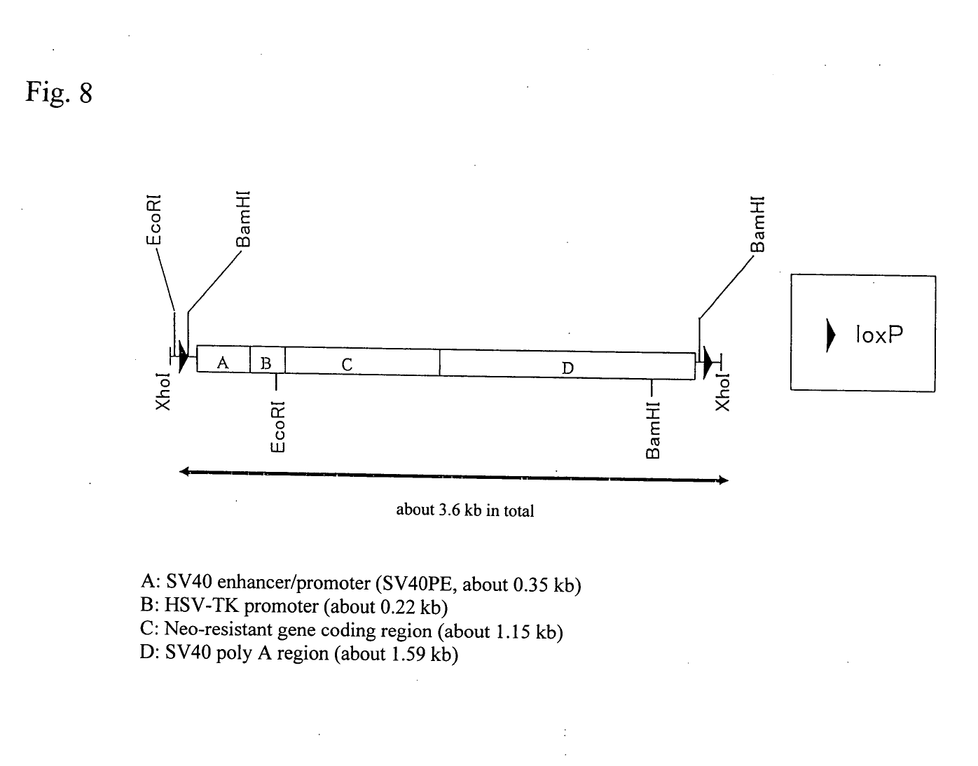

Foreign enhancer may be inserted into a particular site of the chromosome by a gene targeting method. The targeting vector used comprises at least a sequence containing the foreign enhancer, a sequence homologous to the 5′ region upstream of the foreign enhancer insertion site on the chromosome, and a sequence homologous to the 3′ region downstream of the insertion site. Optionally, the targeting vector may further comprise a first gene (also referred to as “first gene”) encoding an exogenous protein, for example, a selection marker gene such as a drug resistant gene (e.g., neomycin resistant gene, puromycin resistant gene, or blasticidin resistant gene). In this case, the foreign enhancer can be introduced into the chromosome in the form of a drug resistant marker gene expression unit comprising the foreign enhancer together with the first gene under the control of the enhancer. The unit may further contain one or more promoters and a poly A signal sequence. More specifically, exemplified is a drug resistant marker gene expression unit of the structure comprising SV40 enhancer/promoter (SV40E/P), HSV-TK promoter, Neo resistant marker gene and SV40 poly A (with a LoxP sequence at both ends) as shown in FIG. 8, wherein HSV-TK represents thymidine kinase from herpes simplex virus. Furthermore, the upstream and downstream genomic regions are normally constituted of a certain number of nucleotides, for example 2 kb or more, desirably 7 kb or more in sum of upstream and downstream nucleotides.

Furthermore, the targeting vector may preferably comprise a negative selection marker. The negative selection marker plays a role inexcluding cells having a random insert of the targeting vector into the genome. Examples of such a negative selection marker include diphtheria toxin A chain gene (DT-A). In the present invention, the negative selection marker is engineered so as not to be exposed at the end of the targeting vector when the targeting vector is linearized. In this manner, the efficiency of homologous recombination can be further improved. For this purpose, in the linearized targeting vector, the 5′ and 3′ ends of the gene structure serving as a negative selection marker are desirably engineered such that they are located at least 1 kb, preferably at least 2 kb, apart from the 5′ and 3′ ends of the targeting vector, respectively. Since a region for homologous recombination with a genome (i.e., homologous recombination region) is usually located at either 5′ end or 3′ end of the negative selection marker, the distance from the end of the vector is 3 kb or more.

The insertion position of a foreign enhancer is a site within 100 Kb, preferably 50 Kb, more preferably 30 Kb, downstream of the 3′ end of the immunoglobulin gene. In a specific example, the insertion position corresponds to the site of the RS element about 25 Kb downstream of the immunoglobulin κ light chain constant region gene on the murine chromosome 6, or a site in its vicinity of the RS element. In this case, foreign enhancer may be inserted into the site of the RS element or a site in its vicinity of the RS element so as to destroy or retain the function of the RS element. The term “the vicinity of the RS element” as used herein refers to a region within approximately several Kb upstream of the 5′ end of the RS element sequence and within approximately several Kb downstream of the 3′ end of the same. The “several Kb” represents 1-10 Kb or less, for example 7 Kb or less, 5 Kb or less, 3 Kb or less, or 1 Kb or less.

The immunoglobulin gene may be either a heavy-chain gene or a light-chain gene. The heavy-chain gene and the light-chain gene (i.e., κ chain or λ chain gene) are present on different chromosomes, each of which has a variable (V) region gene and a constant (C) region gene. In the present invention, the light chain constant region gene is preferably used, and the κ light chain constant region gene is more preferable particularly when pluripotent murine cells are employed. Since genomic analyses of human and mouse among mammals have been virtually completed, genomic information is available at present. As a result of the analyses (i.e., comparison of genomic sequence homology), the human and murine genomes have % homology of about 85%. For these reasons, mouse can preferably be selected as an animal species, and murine pluripotent cells can preferably be used. However, in the invention, animals other than mouse, for example, cow, sheep, pig, hamster, monkey, goat, rabbit, and rat may be used. The immunoglobulin gene sequence of an animal, if the sequencing has been completed, is available from documents or the databases such as the GenBank (NCBI in USA) and EMBL (EBI in Europe). In the case where the sequencing of a gene has not yet been made, it can be determined by combination of fragmentation of genomic DNA with restriction enzymes, mapping, construction of genomic library, cloning, and (automatic) sequencing (Genome Analysis Basic, by S. B. Primrose, translated by Asao Fujiyama, 1996, Shupringer Fairlark Tokyo). The sequence information of an immunoglobulin gene of mouse is available under the GenBank Accession Nos. NG004051 (mouse IgGκ) and VO1569 (mouse IgGκ constant region).

Used as a cell having pluripotency (also referred to as a “pluripotent cell”) in the invention are, as defined above, embryonic stem cell (ES cell), embryonic germ cell (EG cell), and embryonic carcinoma cell (EC cell). Preferably, it is a murine ES cell.

In the invention, examples of a non-human animal include vertebrates such as fish, reptile, amphibian, bird, and mammal preferably mammal. Since ES cell is preferably used as the pluripotent cell in preparing a chimeric non-human animal, non-human animals such as rodents (such as mouse, rat and hamster), cow, sheep, pig, monkey, goat and rabbit, from which the embryonic stem cells can be established, or any other non-human animals from which embryonic stem cells could be established in future, can be used in the invention. A preferable mammal is a rodent, particularly mouse.

The present invention also provides a method of preparing a pluripotent cell derived from a non-human animal and having a foreign enhancer inserted into the particular chromosomal region.

This method comprises preparing a gene targeting vector which comprises a sequence homologous to a 5′ region upstream of a foreign enhancer insertion position of the chromosome of a pluripotent derived from non-human animal cell, a sequence comprising the foreign enhancer, and a sequence homologous to a 3′ region downstream of the insertion position; and introducing the gene targeting vector into the pluripotent cell, thereby integrating a unit comprising the foreign enhancer (e.g., FIG. 8) into a site downstream of an immunoglobulin gene, wherein the insertion position of the foreign enhancer is a site within 100 Kb or less, preferably 50 Kb or less, more preferably 30 Kb or less, from the 3′ end of the immunoglobulin gene.

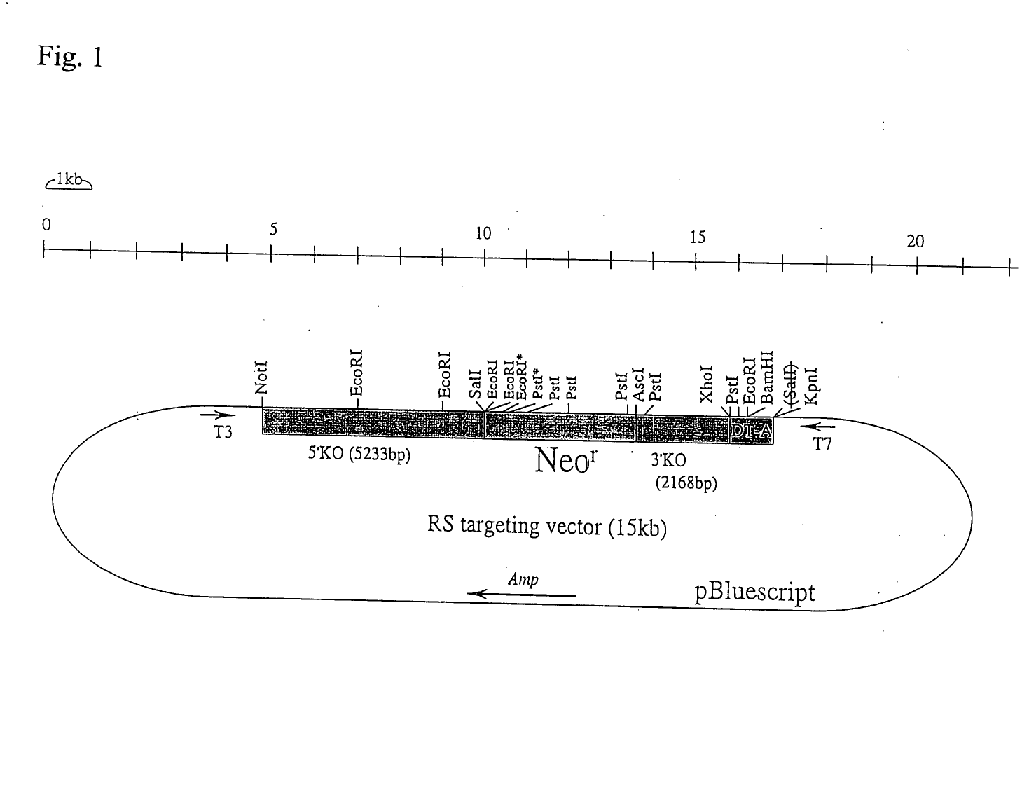

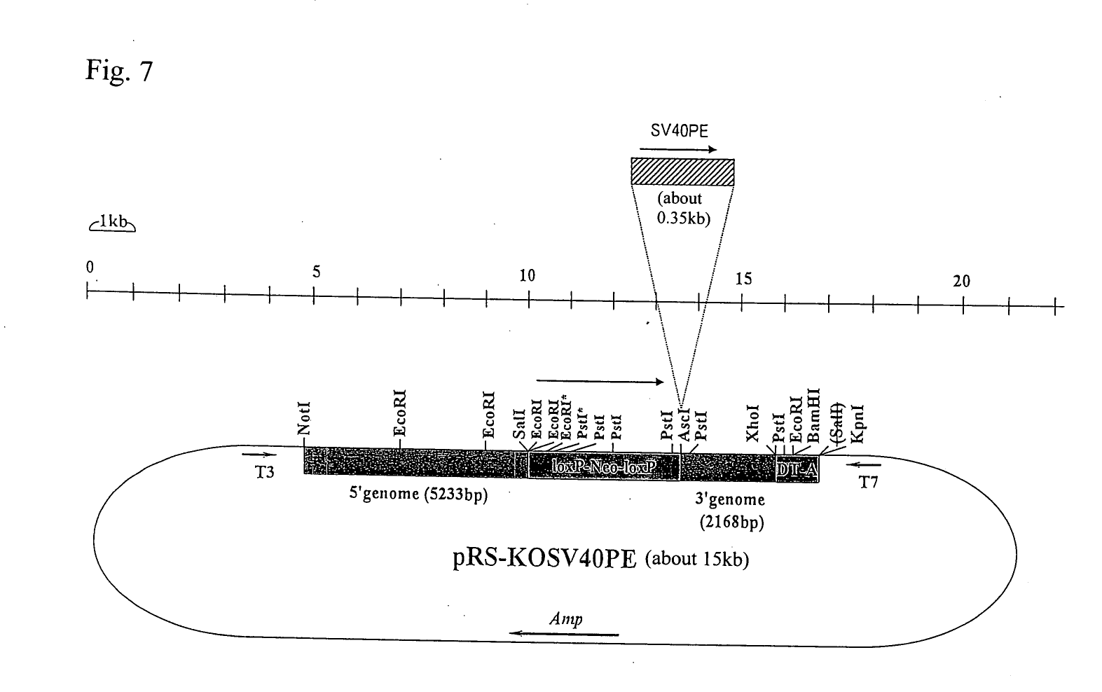

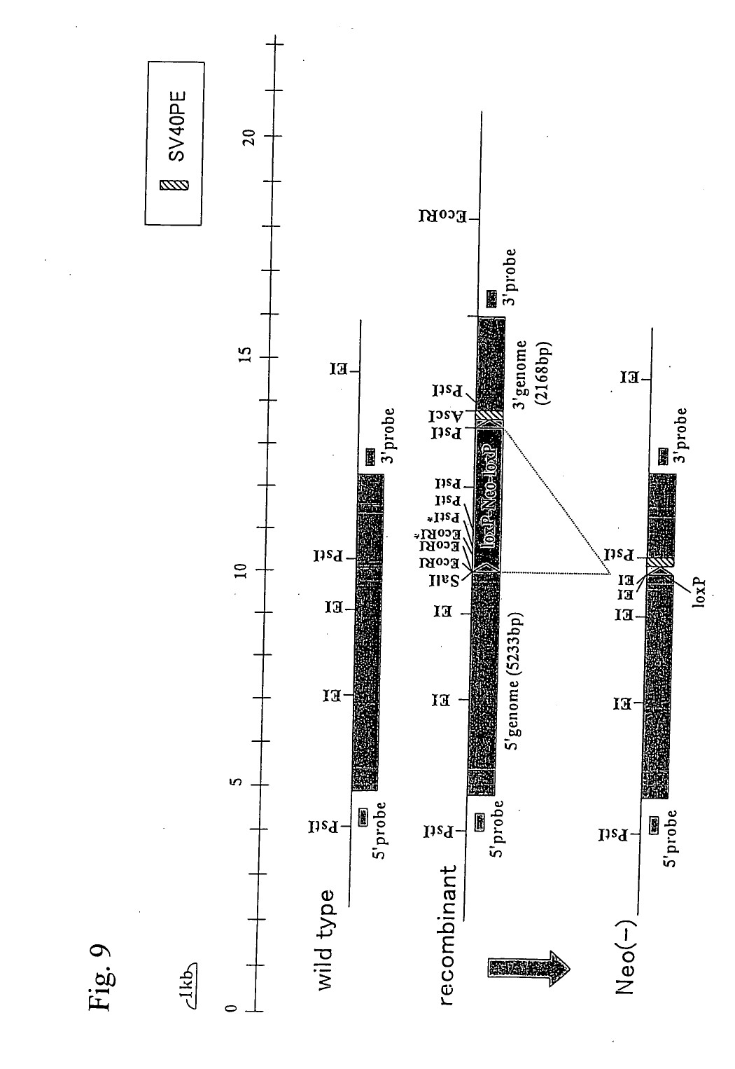

The vector may comprise a first gene under the control of a foreign enhancer. Examples of the first gene may, as defined above, include selection marker genes such as drug resistant genes (e.g., neomycin resistant gene, puromycin resistant gene, blasticidin resistant gene, etc), and negative selection marker genes such as a diphtheria toxin A chain gene. In a specific example of the invention, neomycin resistant gene is used as the first gene. Use as the vector include, but not limited to, plasmid vectors such as PUC plasmids, pBI plasmids and pBluescript plasmids; and phage vectors such as Charon 32, EMBL4 and λZAP. Examples of the vector are pBlueRS-LoxP-Neo-DT-A-3′KO-5′KO shown in FIG. 1, and more specifically, pRS-KOSV40PE shown in FIG. 7. In the figures, 5′KO is the 5′ region upstream of the murine RS element, Neo^{r} is a neomycin resistant gene, 3′KO is the 3′ region downstream of the murine RS element, DT-A is a diphtheria toxin A chain gene, and pBluescript is a cloning vector. The sequence represented by Neo^{r} or loxP-Neo-loxP contains a sequence comprising SV40 enhancer (FIG. 9).

The foreign enhancer, non-human animal, foreign enhancer insertion position, and pluripotent cells are as defined above.

Examples of preferable foreign enhancers include enhancers of an infectious mammalian virus such as SV40 enhancer.

A preferable foreign enhancer insertion position is within 30 Kb or less from the 3′ end of the immunoglobulin gene, for example, the RS element site or in the vicinity thereof.

A preferable non-human animal is a mammal, in particular, a rodent such as mouse.

Preferable pluripotent cells are ES cells, for example, mammalian ES cells, particularly, murine ES cell.

The present invention further provides a method of introducing a gene (also referred to as a “second gene”) encoding a desired protein known or unknown in function, into the chromosome of a pluripotent cell derived from a non-human animal, comprising introducing the second gene in an expressible state by homologous recombination into a site downstream of an immunoglobulin gene and upstream of an foreign enhancer on the chromosome of the pluripotent cell prepared in accordance with the aforementioned method.

According to this method, the rate of homologous recombination when the second gene encoding a desired protein is introduced into the chromosome is 20% or more, 25% or more, 30% or more, 40% or more, 50% or more or 60% or more, which is higher than a conventional rate (about 16%). In this respect, the present invention provides an excellent homologous recombination method of an endogenous or exogenous gene known or unknown in terms of function on the chromosome.

The present invention also provides a pluripotent cell derived from a non-human animal characterized by comprising a foreign enhancer and a second gene as mentioned above on the chromosome, which can be prepared by a method as above. More specifically, the present invention relates to pluripotent cells derived from a non-human animal, characterized by comprising the second gene encoding a desired protein and known or unknown in function, in an overexpressible state, at a site downstream of the immunoglobulin gene and upstream of the foreign enhancer of the chromosome of each of the pluripotent cells.

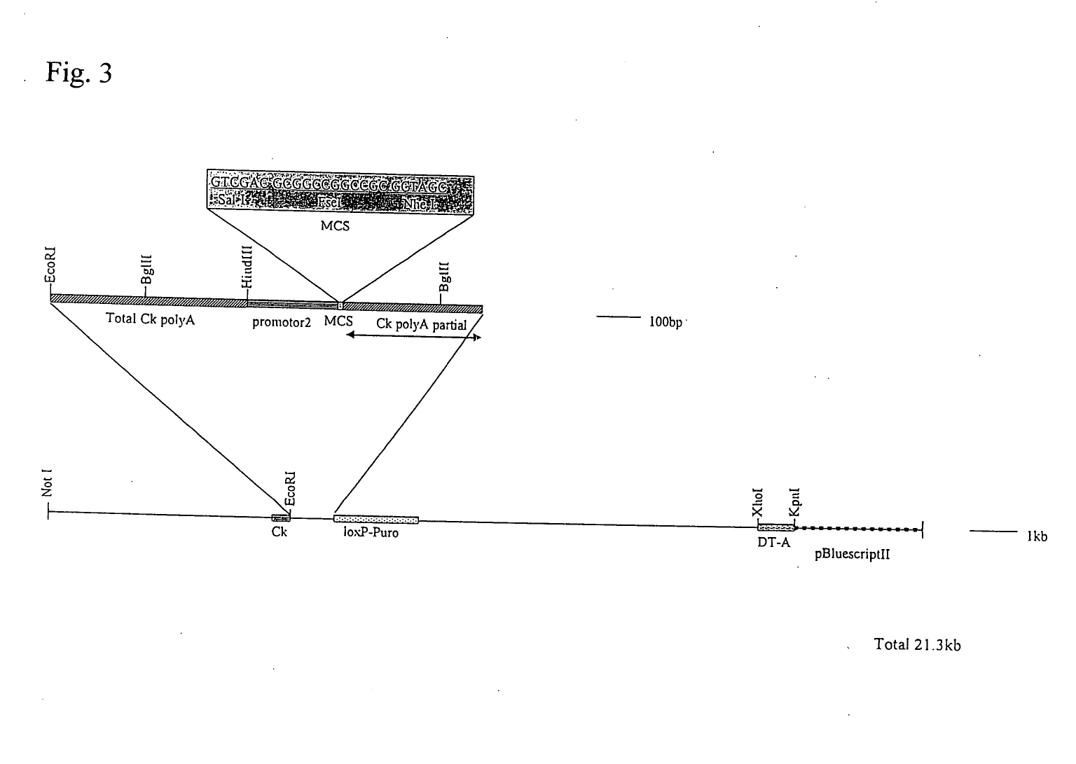

In the invention mentioned above, the second gene encoding a desired protein can be introduced into a chromosome by the gene targeting method. More specifically, the second gene is introduced by using the gene targeting vector, which comprises at least a sequence homologous to the 5′ upstream region of the gene insertion position, a sequence homologous to the second gene sequence, and the 3′ downstream region of the gene insertion position of the chromosome. The sequences of the upstream and downstream genomic regions are satisfactory if each consists of not less than a certain number of nucleotides. For example, it is desirable that each of the upstream and downstream genomic regions may have 2 kb or more and they have 7 kb or more in total. These genomic sequences are available from the databases such as the GenBank (NCBI in USA) and EMBL (EBI in Europe) and can be amplified by PCR using specific primers with these sequences as templates. The PCR is performed using a heat resistant polymerase such as Taq polymerase (Takara Shuzo, Japan), AmpliTaq (Perkin Elmer), and Pfu polymerase (Stratagene) by repeating a cycle consisting of a denaturation step of 80 to 100° C. for 5 seconds to 2 minutes, an annealing step of 40 to 72° C. for 5 seconds to 5 minutes, and an elongation step at 65 to 75° C. for 30 second to 10 minutes, 10 to 40 times. The vector may appropriately contain, other than the aforementioned elements, a promoter for controlling expression of said second gene, a multicloning site (or sequence) for integrating the second gene, a poly A signal sequence which is a control sequence for adding poly A to the 3′ end of a transcript after the transcription of the second gene, a selection marker sequence for confirming whether a desired gene in integrated or not, and a negative marker sequence (e.g., a diphtheria toxin A chain gene). Examples of such a vector include, but not limited to, plasmid vectors such as PUC series plasmids, pBI series plasmids and pBluescript series plasmids; and phage vectors such as Charon 32, EMBL4 and λZAP. The vector specifically usable in the invention is CκP2 knock-in vector having the structure shown in FIG. 3, where Promoter 2 is murine Igκ promoter region gene 2, MCS is a multicloning site, Cκ is a murine Igκ gene constant region, Cκ poly A is a polyA signal region of the murine Igκ, Puro is a puromycin resistant gene, DT-A is a diphtheria toxin A chain gene, and pBluescript is a cloning vector. The second gene is inserted into the multicloning site as shown in FIG. 3.

The non-human animal is selected from vertebrates, preferably a mammal, and more preferably a rodent, particularly a mouse.

As the pluripotent cells and the foreign enhancer, those exemplified above may be used.

The immunoglobulin gene may be either a variable region gene or a constant region gene of the heavy chain (e.g., μ, γ, α, δ, or ε) or light chain (e.g., κ or μ), preferably a heavy-chain or light-chain constant region gene, more preferably a light-chain constant region gene, and most preferably a κ-light chain constant region gene.

The present invention further provides a method of preparing a chimeric non-human animal characterized in that the second gene is overexpressed, comprising injecting a pluripotent cell derived from a non-human animal comprising a second gene introduced in the aforementioned manner, into a host embryo, and transplanting the host embryo to a cognate surrogate mother via injection, and permitting the surrogate mother to give birth.

More specifically, this method comprises injecting the pluripotent cell into the blastocyst stage or 8-cell stage embryo of a non-human animal devoid of certain cells and/or tissue, transplanting the blastocyst stage or 8-cell stage embryo to the surrogate mother of non-human animal, and permitting the surrogate mother to give birth to obtain a chimeric non-human animal. The chimeric non-human animal is preferably a mouse. Examples of such a non-human animal host embryo devoid of certain cells and/or tissue is a B-cell defective host embryo.

The present invention further provides a chimeric non-human animal, which is prepared by the method as mentioned above. The chimeric non-human animal of the present invention is characterized in that the second gene is overexpressed. The preferable animal usable in the invention is a rodent, particularly mouse.

The present invention further provides a progeny of the non-human animal prepared by crossing the chimeric non-human animals and is characterized in that the second gene is overexpressed. The preferable animal is a rodent, particularly a mouse. The crossing is performed between a chimeric non-human animal as prepared above and a cognate non-human animal, thereby obtaining a transgenic (Tg) animal that is a heterozygote in relation to the transgene. Further, when a male and a female of the obtained Tg animals are crossed, a chimeric non-human animal that is a homozygote in relation to the transgene, and further progeny of the non-human animal having the transgene inherited from the parent can be created.

The present invention further provides a method of analyzing the function of a second gene or a protein encoded by the second gene, comprising comparing the difference in phenotype between the second gene overexpressed in the chimeric non-human animal or its progeny with that of a control chimeric non-human animal derived from wild-type pluripotent cell, and analyzing the function of a second gene or a protein encoded by the second gene. The preferable animal is a rodent, particularly mouse. The difference in phenotype can be evaluated based on appearance, biological/hematological features, and pathological observations (e.g., dysfunction, hyperfunction, or behavioral abnormality).

The present invention further provides a method of producing a useful protein by expressing the second gene encoding a desired protein by use of a chimeric non-human animal or its progeny. The preferable animal is a rodent, preferably mouse. This method comprises producing a useful protein using any one of the tissue or cells of the animal or hybridomas; and recovering the protein. Examples of the tissue or cell include the lymph tissue or B cell, respectively. Furthermore, examples of the hybridomas include hybrid cells between B cells or spleen cells including B cells and myeloma cells.

Now, the present invention will be more specifically described below by way of examples, which are provided for facilitating understanding of the invention and thus should not be construed as limiting the invention.

1. Preparation of Pluripotent Cells Derived from a Non-Human Animal Having a Nucleic Acid Sequence Encoding a Desired Protein in a Certain Chromosomal Region

In the method of producing a chimeric non-human animal according to the present invention, pluripotent cells are first prepared, which are derived from a non-human animal and comprise a genome in which a nucleic acid sequence (also called a structural gene, a transgene, or a second gene herein) encoding a desired protein has been located. The nucleic acid sequence is arranged such that the expression of the desired protein (encoded by the nucleic acid) can be controlled by the control region of a gene expressed in the certain cell and/or tissue.

The gene expressed in the certain cell and/or tissue may be expressed tissue-specifically or constitutively. Examples of such a gene expressed tissue-specifically include immunoglobulin light chain or heavy-chain gene, T cell receptor gene, myoglobin gene, crystalline gene, rennin gene, lipase gene, and albumin gene. Examples of such a gene constitutively expressed include hypoxanthine guanine phosphoribosyl transferase (HPRT) gene. When the gene is expressed in a non-human animal tissue-specifically, an embryo devoid of the cell and/or tissue expressed by the gene can be employed as the host embryo (described later). When the gene is constitutively expressed, the embryo to be employed may be devoid of any cell and/or tissue.

The arrangement (alternatively, ligation or insertion) of a nucleic acid sequence (a structural gene or a second gene) encoding a desired protein is required to be performed such that the expression of the desired protein encoded in the nucleic acid sequence can be controlled at least by the control region of the gene to be expressed in certain cell and/or tissue. Accordingly, the nucleic acid sequence is arranged downstream of the control region of the gene to be expressed in certain cells and/or tissue.

Alternatively, the nucleic acid sequence (a structural gene or a second gene) encoding a desired protein is arranged as follows; an internal ribosomal entry site (IRES) is interposed between the termination codon of the gene to be expressed in certain cells and/or tissue and a sequence encoding a poly A signal region, and the nucleic acid sequence (a structural gene or a second gene) encoding a desired protein is arranged downstream of the IRES. More specifically, the nucleic acid sequence is present between the termination codon of the gene to be expressed in certain cells and/or tissue and the sequence encoding the poly A signal region while being functionally ligated with the IRES in a genomic level. The poly A signal region usable in constructing a targeting vector includes, but is not limited to, a poly A signal region of the gene to be expressed in certain cells and/or tissue, or another poly A sequences known in the art such as poly A signal region derived from simian virus 40 (SV40).

Alternatively, a nucleic acid sequence (a structural gene or a second gene) encoding a desired protein may be arranged as follows: a sequence encoding a second poly A signal region is arranged between the termination codon of the gene to be expressed in certain cells and/or tissue and the aforementioned sequence encoding the poly A signal region; a promoter sequence is arranged downstream of the second poly A signal region; and the nucleic acid sequence (a structural gene or a second gene) encoding a desired protein is arranged downstream of the promoter sequence. More specifically, the nucleic acid sequence is present on genome while being functionally ligated with the promoter sequence and the sequence encoding the poly A signal region; at the same time, a gene(s) originally present on the genome and expressed in certain cells and/or tissue is also functionally ligated with the promoter sequence and the sequence encoding the poly A signal region. The promoter sequence used in constructing the targeting vector is not particularly limited as long as it controls the expression of a gene in certain cells and/or tissue. Preferably, use may be made of the promoter for the gene to be expressed in the aforementioned certain cells and/or tissues. Where two promoters are present in a targeting vector, these two promoters may be the same or different as long as they control the expression of the gene in the same cells and/or tissue. Furthermore, the sequence encoding the poly A signal region used in constructing a targeting vector is not particularly limited as long as it is a sequence encoding a known poly A signal region in the art. Examples of the poly A signal region include a poly A signal region derived from the same origin of the promoter or a poly A signal region derived from simian virus 40 (SV40). As in the promoter, when two sequences encoding poly A signal regions are present in the targeting vector, these sequences may be the same or different.

Furthermore, the nucleic acid sequence (structural gene or a second gene) encoding a desired protein may be arranged downstream of a poly A signal region of the gene to be expressed in the certain cells and/or tissue in the order of the promoter sequence, the nucleic acid sequence, and the sequence encoding a poly A signal region. More specifically, the nucleic acid sequence may be present downstream of the poly A signal of the gene to be expressed in the certain cells and/or tissue while it is functionally ligated (in a cassette format) to both the promoter and the sequence encoding the poly A signal region. The promoter sequence used in constructing a targeting vector is not particularly limited as long as it controls the expression of the gene in certain cells and/or tissue. Preferably, use may be made of the promoter of the gene to be expressed in the aforementioned certain cells and/or tissue. Furthermore, the sequence encoding the poly A signal region in constructing a targeting vector is not particularly limited as long as it is the sequence of a known poly A signal region in the art. Examples of the poly A signal region include a poly A signal region derived from the same origin as the promoter and a poly A signal region derived from simian virus 40 (SV40). When there are two sequences encoding poly A signal regions in a targeting vector, they may be the same or different. The distance between the 3′ end of the poly A signal region of the gene to be expressed in the certain cells and/or tissue and the 5′ end of a promoter sequence controlling the expression of a nucleic acid sequence encoding a desired protein is not particularly limited as long as the nucleic acid sequence can be expressed in the certain cells and or tissue. However, as the distance increases, the stability of a transcript, mRNA, may be undesirably affected. In addition, the size of the structure of a targeting vector becomes larger. As a result, it is difficult to construct such a vector. For these reasons, it is preferable that the distance between the 3′ end of the poly A signal region and the 5′ end of the promoter sequence controlling the expression of the nucleic acid sequence encoding a desired protein preferably falls within 1 Kb.

When a nucleic acid sequence (structural gene or a second gene) encoding a desired protein is arranged in the vicinity of the control region, the nucleic acid sequence may be inserted in the vicinity of the control region, or may be arranged immediately downstream of the control sequence of the gene to be expressed in the certain cells and/or tissue in such a manner that it replaces an original structural gene, with respect to one of the alleles of a pluripotent cell such as ES cell. To explain more specifically, since the original structural gene replaced by the nucleic acid sequence encoding a desired protein can be expressed by the other allele, the cells and/or tissue can remain normal. However, in a gene (e.g., immunoglobulin gene) where allelic exclusion occurs, the nucleic acid sequence encoding a desired protein can be arranged as follows: the IRES sequence is arranged downstream of the termination codon of the original structural gene in both alleles in a pluripotent cell such as ES cell, and the nucleic acid sequence encoding a desired protein is arranged downstream of the IRES sequence. Alternatively, one allele for the original structural gene may be previously inactivated in the pluripotent cell such as ES cell, and thereafter, the IRES sequence may be allowed to intervene downstream of the termination codon of the original structural gene in the allele not inactivated, and the nucleic acid sequence encoding a desired protein may be arranged downstream of the IRES sequence. In this case, it is expected that the allele not inactivated is exclusively expressed; at the same time, the nucleic acid encoding a desired protein is expected to be expressed at a high level.

An immunoglobulin κ chain gene is expressed by joining many V and J gene segments recombinantly, as mentioned above. As a result of the joining, the promoter sequence present in the vicinity of the upstream region of each V gene segment comes in the vicinity of an enhancer sequence present downstream of J gene segments. The enhancer sequence cannot activate the promoter until such an arrangement takes place (Picard et al., Nature, 307:80-2, 1984). More specifically, the nucleic acid sequence encoding a desired protein can be arranged in the vicinity of the enhancer sequence by artificially linking it to the promoter sequence of the immunoglobulin κ chain gene. It is known that another enhancer sequence is present further downstream in the immunoglobulin κ chain gene locus (Meyer et al., EMBO J. 8: 1959-64, 1989). Likewise, the gene is highly expressed in B cells under the influence of a plurality of enhancers.

The pluripotent cells such as ES cells derived from a non-human animal and containing a genome having a nucleic acid sequence encoding a desired protein as mentioned above can be obtained as described below.

2. Obtaining ES Cells with Transferred Gene

(1) Construction of Targeting Vector

To introduce a sequence containing a foreign enhancer at a site downstream of an immunoglobulin gene in the chromosome of a non-human animal, a targeting vector is constructed. The targeting vector comprises genomic sequences corresponding to the upstream and downstream regions of a foreign enhancer insertion position, and the foreign enhancer and a selective marker under the control of the enhancer, which are inserted between the genomic sequences. The foreign enhancer insertion position is about 25 Kb downstream of a murine immunoglobulin κ chain gene, that is, the position of an RS element or in the vicinity thereof, in an Example of the present invention.

Each of the genome sequences corresponding to the upstream and downstream regions of the foreign enhancer insertion position may be constituted of a certain number of nucleotides, for example, desirably 2 kb or more and the total of the upstream and downstream genome sequences is desirably 7 kb or more.

Examples of the usable selective marker include neomycin resistant gene, puromycin resistant gene, blasticidin resistant gene, GFP gene, and the like.

Furthermore, the structure of a targeting vector may be modified in order to improve the homologous recombination efficiency. More specifically, the homologous recombination efficiency can be increased by engineering a negative selection marker for excluding cells with targeting vectors randomly inserted into the genome so as not to be exposed at the end(s) of the vector when the targeting vector is linearized.

More specifically, in the targeting vector linearized, a gene serving as a negative selection marker is desirably engineered such that its 5′- and 3′-ends are positioned at least 1 Kb, preferably 2 Kb or more apart from the 5′- and 3′-ends of the targeting vector, respectively. Since a region utilized for homologous recombination with a genome (i.e., homologous recombination region) is usually located at one of the 5′ end and the 3′ end of a negative selection marker, the distance from the one of the ends of the vector comes to be 3 Kb or more, in most cases. On the other hand, the other end of the negative selection marker is often arranged close to the other end of the vector. In the present invention, the negative selection marker is engineered such that the end of the negative selection marker, which is not arranged next to the homologous recombination region, is arranged at a distance of at least 1 kb apart from the end of linearized vector. In this manner, homologous recombination efficiency is increased. As the sequence to ensure the distance from the end of the vector, the sequence of a plasmid vector such as pUC, which is employed in constructing a targeting vector, may be used as it is (without removing when the targeting vector is linearized). Alternatively, as the sequence, a new non-coding sequence not homologous to a desired targeting region may be arranged next to the negative selection marker. The vector is linearized by probing restriction enzyme recognition sites of the targeting vector in use and selecting an appropriate restriction recognition site, thereby ensuring a proper distance between the vector end and the negative selection marker end. In this manner, the effect of improving homologous recombination efficiency can be attained. Even if such an appropriate restriction site is not found, an appropriate restriction enzyme recognition sequence can be introduced into a desired position of a targeting vector by a method using PCR (Akiyama et al., Nucleic Acids Research, 2000, Vol. 28, No. 16, E77.).

It is suggested that taking the structure of a targeting vector as mentioned above allows the frequency of attacks of the negative selection marker to a nuclease to reduce in a cell, thereby elevating the efficiency of homologous recombination.

In short, the present invention provides a gene targeting vector characterized in that the 5′- and 3′-ends of a gene structure functioning as a negative selection marker are apart from at least 1 Kb, preferably 3 Kb or more from the 5′ end and 3′ end of the linearized targeting vector respectively, and provides a method for targeting a gene using the targeting vector. In the targeting vector, any negative selection vector may be used as long as it is known in the art. Preferably, diphtheria toxin A chain gene may be used as the negative selection marker.

(2) Obtaining Non-Human Animal Pluripotent Cells to be Targeted

Non-human animal pluripotent cells (e.g., murine ES cell) can be usually established by the method as described below. Male and female non-human animals are crossed. The 2.5-day old embryo after fertilization is taken and cultured in vitro in culture medium for pluripotent cells. The embryo developed till the blastocyst stage is separated from the cultured embryos, and is seeded ans cultured on a medium with feeder cells. From the cultured embryos, embryos growing in a pluripotent cell like form are selected. A cell mass is taken from the embryos thus selected, dispersed in the medium for ES cells containing trypsin, cultured in the medium with feeder cells, and then, sub-cultured in the medium for pluripotent cells. The grown cells are isolated.

An RS element targeting murine ES cell can be obtained by use of a targeting vector in accordance with any method known in the art as described in, for example, Bio-Manual Series 8, Gene Targeting (by Shinichi Aizawa), 1995, Yodosha, Japan. More specifically, the targeting vector as constructed above is introduced into murine ES cells by electroporation or lipofection to obtain murine ES cells devoid of the RS element and having a resistant gene inserted into the deleted region. Through the procedures as mentioned above, it is possible to obtain murine ES cells enhanced in homologous recombination efficiency in a chromosomal region downstream of the immunoglobulin light chain constant region gene.

(3) Construction of Targeting Vector

First, a targeting vector is constructed in such a manner that it comprises a gene to be expressed in certain cells/or tissue, a promoter region thereof in the vicinity of the gene, and a nucleic acid sequence encoding a desired protein inserted downstream of the promoter portion.

As the nucleic acid sequence to be introduced, cDNA or genomic DNA containing an intron(s) may be used as long as it comprises a sequence from initiation codon to termination codon. The type of the protein encoded by the nucleic acid sequence may not be limited. The nucleic acid sequence to be used in the present invention may be used for highly expressing/secreting the protein encoded by the nucleic acid sequence or for elucidating the function of the protein. Accordingly, as long as the nucleotide sequence can be specified, any type of the nucleic acid sequence may be used. Examples of such a nucleic acid sequence (or structural gene) include nucleotide sequences of genes encoding functional proteins derived from a mammal, preferably a human, such as genes encoding secretory proteins, genes encoding membrane proteins, and genes encoding intracellular or intranuclear proteins.

The nucleic acid sequence encoding a desired protein may have a promoter sequence, a nucleic acid sequence and a sequence encoding a poly A signal region, which are arranged in order downstream of the poly A signal region of the gene to be expressed in the certain cells and/or tissue as mentioned above. In other words, the nucleic acid sequence is operably linked to the promoter and the sequence encoding a poly A signal region (in a cassette format) and is present downstream of the poly A signal of the gene to be expressed in the certain cells and/or tissue. The promoter sequence used in constructing a targeting vector is not particularly limited as long as it controls the expression of the aforementioned specific gene in certain cells and/or tissue. Preferably, promoters for genes expressed in the aforementioned specific cells and/or tissue can be used. The sequence encoding a poly A signal region used in constructing the targeting vector is not particularly limited as long as it is a known poly A signal region in the art. Examples of such a poly A signal region include a poly A signal region derived from the same origin as the promoter, and a poly A signal region derived from simian virus 40 (SV40). When two sequences encoding poly A signal regions are present in the targeting vector, they may be the same or different. The distance between the 3′ end of the poly A signal region of the gene to be expressed in the certain cells and/or tissue and the 5′ end of the promoter sequence controlling the expression of the nucleic acid sequence encoding a desired protein is not particularly limited as long as the nucleic acid sequence can be expressed in the certain cells and/or tissue. As the distance increases, the stability of a transcript, mRNA, may be undesirably affected. In addition, the size of the structure of a targeting vector becomes larger. As a result, it is difficult to construct such a vector. For these reasons, it is preferable that the distance between the 3′ end of the poly A signal region and the 5′ end of the promoter sequence controlling the expression of the nucleic acid sequence encoding a desired protein preferably falls within 1 Kb.

To modify an animal genome so as to contain a nucleic acid sequence encoding a desired protein in the vicinity of the gene to be expressed in certain cells and/or tissue, or alternatively so as to contain a promoter of the gene to be expressed in certain cells and/or tissue in the vicinity of the gene and further a nucleic acid sequence encoding a desired protein downstream of the promoter, a targeting vector is provided. The nucleic sequence encoding the desired protein may be inserted into the targeting vector DNA. Examples of such a targeting vector for this purpose include plasmids and viruses. It is easy for a skilled person in the art to select and obtain a vector suitably usable as such a targeting vector. Such a vector includes, but is not limited to, a CκP2 targeting vector (see Examples described later). In the targeting vector, an appropriate restriction enzyme cleavage site serving as a desired nucleic acid sequence (DNA) insertion site is inserted (e.g., near the middle point) between the termination codon of the gene to be expressed in certain cells and/or tissue and the poly A addition site. Into the restriction enzyme cleavage site, DNA (cDNA or genomic DNA) containing the initiation codon to the termination codon of the nucleic acid sequence to be introduced, is inserted. Also, in this case, a translation promoting sequence, such as Kozak sequence, may be arranged preferably upstream of the initiation codon. Furthermore, if necessary, the vector may comprise a selection marker such as puromycin resistant gene, neomycin resistance gene, blasticidin resistant gene, or GFP gene.

(4) Introduction of a Targeting Vector into Pluripotent Cells Derived from a Non-Human Animal and Selection of Homologous Recombinants

Pluripotent cells derived from a non-human animal each can be transformed by a targeting vector in accordance with a known method in the art, for example, described in Bio-Manual Series 8, Gene Targeting (by Shinichi Aizawa), 1995, Yodosha, Japan. More specifically, the targeting vector as constructed above may be introduced into each of the pluripotent cells by electroporation or lipofection.

Moreover, the targeting vector may be modified to increase the efficiency of homologous recombination. More specifically, the homologous recombination efficiency can be increased by engineering a negative selection marker, which is for excluding cells with targeting vectors randomly inserted into the genome, so as not to be exposed to the ends of the target vector when the vector is linearized.

More specifically, in the linearized targeting vector, the 5′- and 3′-ends of a gene serving as a negative selection marker are desirably engineered such that they are positioned at least 1 Kb, preferably 2 Kb or more apart from the 5′- and 3′-ends of the targeting vector. Since a region utilized for homologous recombination with a genome (i.e., homologous recombination region) is usually located at either one of the 5′ end and the 3′ end of the negative selection marker, the distance from the end of the vector comes to be 3 Kb or more, in most cases. On the other hand, the other end of the negative selection marker is often arranged close to the other end of the vector. In the present invention, the negative selection marker is engineered such that the end of the negative selection marker, which is not arranged next to the homologous recombination region, is located at a distance of at least 1 kb apart from the end of linearized vector. In this manner, homologous recombination efficiency is elevated. As the sequence to ensure the distance from the end of the vector, the sequence of a plasmid vector such as pUC, which is employed in constructing the targeting vector, may be used as it is (without removing when the target vector is linearized). Alternatively, as the sequence, a new non-coding sequence not homologous to a desired targeting region may be arranged next to the negative selection marker. The vector is linearized by probing restriction enzyme recognition sites of the targeting vector in use and selecting an appropriate restriction recognition site, thereby ensuring a proper distance between the vector end and the negative selection marker end. In this manner, the effect of improving homologous recombination efficiency can be attained. Even if such an appropriate restriction site is not found, an appropriate restriction enzyme recognition sequence can be introduced into a desired position of a targeting vector by a method using PCR (Akiyama et al., Nucleic Acids Research, 2000, Vol. 28, No. 16, E77.).

It is suggested that taking the structure of a targeting vector as mentioned above allows the frequency of attacks of the negative selection marker to a nuclease to reduce in a cell, thereby elevating the efficiency of homologous recombination.

In short, the present invention provides a gene targeting vector characterized in that the 5′ end and 3′ end of a gene functioning as a negative selection marker are apart from at least 1 Kb, preferably 2 Kb or more, generally 3 Kb or more from the 5′ end and 3′ end of the linearized targeting vector respectively, and provides a method for targeting a gene using the targeting vector. In the targeting vector, any negative selection vector may be used as long as it is known in the art. Preferably, a diphtheria toxin A chain gene may be used.

Furthermore, the efficiency of inserting a target gene into a site downstream of the Igκ constant region gene can be increased by use of, as a non-human pluripotent cell, an embryonic stem cell (e.g., murine ES cell) having a drug resistant marker inserted into the RS element region about 25 Kb downstream of the Igκ light-chain gene.

To easily identify a homologous recombinant, a drug resistant gene marker may be previously introduced into the position to be targeted by a foreign gene. For example, the murine ES cell TT2F, which is used in Examples of the present specification, is derived from F1 individuals between C57BL/6 line and CBA line. When the sequence of the genomic homologous region contained in a targeting vector is derived from C57BL/6 as previously described (Deng & Capecchi, Mol. Cell. Biol., 12:3365-71, 1992), homologous recombination may conceivably take plate more efficiently in the allele derived from C57BL/6 line in the TT2F cell. In other words, it is possible to insert, for example, a G418 resistant marker, into the allele derived from C57BL/6 line in advance by using a targeting vector containing DNA derived from C57BL/6 line. Then, a targeting vector containing a puromycin resistant marker and genomic DNA derived from C57BL/6 line is introduced into the G418 resistant line thus obtained. In this manner, the puromycin resistant and G418 sensitive line can be obtained. In this line, the G418 resistant gene is removed by homologous recombination between the targeting vector and the gene to be expressed in certain cells and/or tissue, and instead, a structural gene encoding a desired protein and the puromycin resistant marker are introduced. In this manner, an analysis step required for identifying a homologous recombinant, such as Southern analysis, can be eliminated.

After the puromycin resistant clone is picked up, the genomic DNA is prepared and subjected to Southern analysis to identify a homologous recombinant in the same manner as that described in PCT International Application WO 00/10383 (published Mar. 2, 2000) filed by the applicant of the present invention. The puromycin resistant gene in the targeting vector is derived from Lox-P Puro plasmid described in WO 00/10383 and contains a Lox-P sequence at the ends thereof in a forward direction. Therefore, the puromycin resistant gene can be removed from pluripotent cells targeted by the method described in WO 00/10383.

The targeting vector and technique/means for improving homologous recombination efficiency can be applied to all cells capable of introducing a gene and not limited to the case of forming a chimeric animal. For example, the targeting vector and the technique/means for improving homologous recombinant efficiency described in the present specification can be used for destroying or introducing a desired gene in gene therapy directed to a human or human cells (such as blood cells or immune cells).

3. Host Embryos Devoid of Certain Cells and/or Tissue

Next, in a method of preparing a chimeric non-human animal according to the present invention, a host embryo of a non-human animal devoid of the certain cells and/or tissue (hereinafter, also referred to as a “defective host embryo”) is prepared. Examples of such a defective host embryo include a B-cell defective embryo due to knock-out of an immunoglobulin heavy-chain gene, when an immunoglobulin light-chain gene is used as the control region (Tomizuka et al., Proc. Natl. Acad. Sci. USA, 18:722-727, 2000); a T lymphocyte defective embryo due to deletion of a T-cell receptor β-chain when T cell receptor gene is used as the control region (Mombaerts et al., Nature, 360: 225-227, 1992); a muscular tissue defective embryo due to knock-out of the myogenin gene when the myoglobin gene is used as the control region (Nabeshima et al., Nature, 364:532-535, 1993); an embryo derived from a murine mutant, aphakia (ak) line, devoid of crystalline lens when the crystalline gene is used as the control region (Liegeois et al., Proc. Natl. Acad. Sci. USA, 93: 1303-1307, 1996); an embryo devoid of the kidney tissue due to knock-out of the sall 1 gene when the renin gene is used as the control region (Nishinakamura et al., Development, 128: 3105-3115, 2001); an embryo devoid of the liver tissue due to deletion of the c-Met gene when an albumin gene is used as the control region (Bladt et al., Nature, 376: 768-770, 1995); and an embryo defective in the pancreas tissue due to knock-out of the Pdx1 gene when a lipase gene is used as the control region (Jonsson et al., Nature, 371: 606-9, 1994). In the above, preferable defective host embryos are exemplified; however, the defective host embryo that may be used in the present invention is not limited to these.

As to selection of the development stage, genetic background or the like of a host embryo for efficiently producing a chimeric non-human animal, the conditions already specified with respect to the ES cell lines based on research are desirably employed. More specifically, in the case of a mouse, when a chimera is produced from the TT2 cell derived from CBA×C57BL/6 F1 mouse or the TT2F cell (wild color, Yagi et al., Analytical Biochemistry, 214:70-76, 1993), a host embryo desirably has a genetic background of Balb/c (white, available from CLEA Japan), ICR (white, available from CLEA Japan) or MCH (ICR) (white, available from CLEA Japan). Therefore, as a defective host embryo, it is desirable to use a non-human animal embryo (e.g., 8-cell stage) obtained by back-crossing a non-human animal line devoid of certain cells and/or tissue with each of the aforementioned lines.

Since the cells and/or tissue that a host embryo is devoid of is compensated by pluripotent cells in accordance with blastocyst complementation (BC), the defective host embryo may be an embryonic lethal as long as it can develop till the blastocyst stage required for producing a chimeric animal. Such an embryonic lethal appears with a rate of ¼ in theory when animals heterozygous for gene defection are crossed with each other. Therefore, chimeric animals are created by using a plurality of embryos obtained by crossing in accordance with the following procedures and defective embryos are selected as host embryos from the embryos obtained from the chimeric animals. The selection is performed by extracting DNA from the somatic tissue of a chimeric animal and subjecting the DNA to Southern analysis, PCR or the like.

4. Production of Chimeric Embryo and Transplantation into Surrogate Mother.

A chimeric non-human animal is produced from the ES cell line with transferred gene as prepared in Section 1 (“Preparation of pluripotent cells”) in accordance with the method of Shinichi Aizawa (as above). More specifically, the pluripotent cell with transferred gene is injected into the blastocyst or 8-cell stage of a defective host embryo as described in Section 3 (“Host embryos devoid of certain cells and/or tissue”) by use of a capillary or the like. Then, the blastocyst or 8-cell stage embryo is directly transplanted to the oviduct of a cognate surrogate mother, which is a non-human animal, or alternatively it is cultured for a day up to a blastocyst embryo, which is then transplanted to the uterus of a surrogate mother. Thereafter, the surrogate mother is allowed to give birth to obtain a child animal.

5. Expression of the Transferred Gene in Chimeric Non-Human Animals

The child animal is produced in accordance with the section of “Production of chimeric embryo and transplantation into surrogate mother”, from an embryo into which a gene-transferred pluripotent cell was injected. The contribution rate of the pluripotent cell to the child animal can be roughly determined based on the hair color of the child animal. For example, when a gene-transferred cell line from TT2F cell (wild color: dark blown) is injected into a host mouse embryo having MCH(ICR) background (white), the rate of the wild color (dark brown) represents the contribution rate of the pluripotent cell. In this case, the contribution rate indicated by hair color correlates with that of a gene-transferred pluripotent cell in cells and/or tissues other than the deleted ones; however, depending upon the tissue, the contribution rate of the pluripotent cell does not sometimes consistent with that indicated by hair color. On the other hand, only the cells and/or tissues from gene-transferred pluripotent cell are present in the chimeric non-human animal, whereas the deleted cells and/or tissue from host embryo do not exist therein. The restoration of the cells and/or tissue deleted in the chimeric non-human animal by contribution of the gene-transferred pluripotent cell can be detected by the FACS (Fluorescence-Activated Cell Sorter) assay, ELISA (Enzyme-linked Immuno Sorbent Assay), or the like. Whether a nucleic acid sequence (or structural gene) inserted into the cells and/or tissue from the gene-transferred pluripotent cell is expressed is detected by the RT-PCR method (Kawasaki et al., P.N.A.S., 85:5698-5702, 1988) using RNA derived from the cells and/or tissue, Northern blot method (Ausubel et al., Current protocols in molecular biology, John Wiley & Sons, Inc., 1994), or the like. When a specific antibody to a desired protein encoded by the transferred nucleic acid sequence is already present, he expression of the protein can be detected by the Enzyme-linked Immuno Sorbent Assay using chimeric mouse serum (ELISA; Toyama and Ando, Monoclonal Antibody Experimental Manual, 1987, Kohdansha Scientific, Japan), Western blot (Ausubel et al., as above), or the like. Alternatively, if DNA encoding the nucleic acid sequence (or structural gene) to be transferred is appropriately modified previously such that a tag peptide detectable with an antibody is added to the protein encoded by the DNA, then the expression of the transferred gene can be detected with the antibody to the tag peptide or the like (e.g., POD labeled anti-His₆; Roche Diagnostics).

In the chimeric non-human animal prepared as described above, the transferred nucleic acid sequence (i.e., a structural gene or second gene) can be highly expressed at least in certain cells and/or tissue. If the desired protein expressed is a secretory protein like blood or milk, the chimeric non-human animal can be used as a production system for a useful protein. Alternatively, if a protein with unknown function is highly expressed, the function of the protein may be elucidated from findings accompanied with the high expression.

Furthermore, recently, the combination of the method for producing animal individuals from somatic cell nucleus-transplanted embryos with the gene targeting in somatic cell has made the gene modification possible as in mouse even in animal species (cow, sheep, pig, etc.) other than mouse (McCreath et al., Nature, 405: 1066-1069, 2000). For example, a cow devoid of B cells can be produced by knocking out an immunoglobulin heavy chain. Alternatively, a certain gene can be inserted into an Ig gene or in the vicinity thereof from an animal such as a mouse, cow, sheep or pig, and subsequently the nucleus comprising the certain gene can be removed from the fibroblast of the animal to transplant into an unfertilized, denucleated egg, which is then developed into a blastocyst stage embryo to prepare an ES cell. From the ES cells thus obtained and the B cell defective host embryo as mentioned above, a chimeric non-human animal can be produced (Cibelli et al., Nature Biotechnol., 16: 642-646, 1998). High expression of secretory proteins using a similar expression system is also possible not only in a mouse but also in other animal species. When a larger animal is used, production of a useful substance becomes possible in addition to analysis of the function of a gene.

6. Production of Progeny of Chimeric Non-Human Animal

The method of producing a chimeric non-human animal according to the present invention further comprises: crossing a chimeric non-human animal with a cognate non-human animal to produce transgenic animals; selecting from the transgenic animals, male and female transgenic (Tg) animals heterozygous for the transferred nucleic acid sequence; crossing the male and female Tg animals to each other to obtain Tg animal progeny homozygous for the transferred nucleic acid sequence (i.e., homozygote) (Transgenic Animal, edited by Kenichi Yamamura et al., 1995, Kyoritsu Shuppan, Japan.).

7. Tissues or Cells Derived from a Chimeric Non-Human Animal or its Progeny

According to the present invention, it is possible to obtain tissues or cells derived from any one of the chimeric non-human animals or progenies thereof as mentioned above. The cells or tissues contain a genome in which a nucleic acid sequence encoding a desired protein is arranged such that the desired protein can be expressed under the control of a control region of a gene expressed in the cells or tissues and thus can express the desired protein.

Any tissue or cell may be used as long as it is derived from a chimeric non-human animal or its progeny and is capable of expressing a desired protein. Examples of such a tissue or cell include B cells, spleen and lymph tissue.

The tissues or cells can be taken and cultured in accordance with a known method in the art. Whether the tissues or cells express a desired protein can also be confirmed by conventional methods. Such tissues or cells are useful for producing a hybridoma or protein as mentioned below.

8. Production of Hybridoma

In the present invention, cells of a chimeric non-human animal capable of expressing a transferred nucleic acid sequence encoding the desired protein (in particular, B cell or spleen cells containing B cell, and cells from lymph tissue such as lymph node) are hybridized with a proliferable tumor cell (e.g., myeloma cell) to obtain hybridomas. A method of producing hybridomas may be based on procedures as described, for example, in the Andoh and Chiba, Introduction of Monoclonal Antibody Experimental Manipulation, 1991, Kohdansha Scientific, Japan).

Used as such a myeloma are for example cells with no ability to produce self-antibodies derived from a mammal such as a mouse, rat, guinea pig, hamster, rabbit, or human, preferably cell lines generally obtainable from mice, such as myeloma cell lines derived from 8-azaguanine resistant mice (BALB/c) P3X63Ag8U.1(P3-U1) [Yelton, D. E. et al., Current Topics in Microbiology and Immunology, 81: 1-7(1978)]; P3/NSI/1-Ag4-1 (NS-1) [Kohler, G. et al., European J. Immunology, 6:511-519 (1976)]; Sp2/O-Ag14 (SP-2) [Shulman, M. et al., Nature, 276:269-270 (1978)]; P3X63Ag8.653(653) [Kearney, J. F. et al., J. Immunology, 123:1548-1550 (1979)]; and P3X63Ag8 (X63) [Horibata, K. and Harris, A. W. Nature, 256:495-497 (1975)]. These cell lines are subcultured in an appropriate medium such as 8-azacuanine medium [RPMI-1640 medium containing glutamine, 2-mercaptoethanol, gentamicin, and fetal calf serum (hereinafter refers to as “FCS”) supplemented with 8-azaguanine], Iscove's Modified Dulbecco's medium (hereinafter referred to as “IMDM”), or Dulbecco's Modified Eagle Medium (hereinafter referred to as “DMEM”). However, 3 to 4 days before cell fusion, they are subcultured in normal medium (e.g., DMEM medium containing 10% FCS). In this manner, at least 2×10⁷ cells are prepared until the day of cell fusion.

Used as cells capable of expressing a desired protein encoded by the transferred nucleic acid sequence are for example plasma cells and lymphocytes as the precursor cells, which may be obtained from any part of an animal individual and generally obtained from the spleen, lymph node, bone marrow, amygdale, peripheral blood or an appropriate combination thereof. Generally, spleen cells can be used.

The most general means for fusing a spleen cell, which expresses a desired protein encoded by the transferred nucleic acid sequence, with a myeloma cell is a method using polyethylene glycol since cytotoxicity is relatively low and fusion is simple. More specifically, the fusion can be performed as follows. First, spleen cells and myeloma cells are washed well with a serum free medium (e.g., DMEM) or phosphate buffered saline (generally referred to as “PBS”), mixed in a cell ratio of about 5:1 to about 10:1, and they are centrifugally separated. The supernatant is removed and precipitated cells are loosened. To the loosened cells, the serum free medium containing 1 ml of 50% (w/v) polyethylene glycol (molecular weight 1,000 to 4,000) is added dropwise while stirring. Thereafter, 10 ml of the serum free medium is gently added to the mixture and centrifugally separated. The supernatant is discarded and precipitated cells are resuspended in a normal medium (generally referred to as “HAT medium”) containing hypoxanthine, aminopterin and thymidine and further human interleukin-6 in appropriate amounts, dispensed to wells of a culture plate, cultured at 37° C. for 2 weeks in the presence of 5% carbon dioxide gas while supplying HAT medium appropriately during the culture.

When the myeloma cell is from a 8-azaguanine resistant cell line, namely a hypoxanthine guanine phosphoribosyl transferase (HGPRT) defective cell line, myeloma cells not hybridized and myeloma-myeloma hybrid cells cannot survive in the HAT-containing medium. In contrast, spleen-spleen hybrid cells or spleen cell/myeloma cell hybrids can survive; however, spleen cell/spleen cell hybrids have a limited life. Therefore, if culture is continued in the HAT-containing medium, only spleen cell/myeloma cell hybrids can survive.

The obtained hybridomas can be further screened by ELISA using a specific antibody against the desired protein encoded by the transferred nucleic acid sequence. As a result, hybridoma producing the desired protein encoded by the transferred nucleic acid sequence can be selected.

9. Method of Producing Desired Useful Proteinaceous Substances

The present invention further provides a method of producing a desired protein comprising producing the desired protein by using any one of the chimeric non-human animal or its progeny as described above, the tissues or cells as described above, and hybridomas as described above, followed by recovering the desired protein. More specifically, the chimeric non-human animal or its progeny is kept under the conditions in which the transferred nucleic acid sequence encoding a desired protein can be expressed, and the protein, an expression product, is recovered from the blood, ascite fluid or the like of the animal. Alternatively, a tissue or cells derived from a chimeric non-human animal or its progeny or the tissue or cells immortalized (for example, hybridomas immortalized by fusing them with myeloma cells) are cultured under such conditions that the transferred nucleic acid sequence encoding a desired protein can be expressed, and thereafter, the protein, an expressed product, is recovered from the culture or the supernatant thereof. The expressed product can be recovered by using a known method such as centrifugation and further purified by using known methods, such as ammonium sulfate fractionation, partition chromatography, gel filtration chromatography, absorption chromatography (e.g., ion exchange chromatography, hydrophobic interaction chromatography, or affinity chromatography), preparative thin-layer chromatography, and HPLC, alone or in combination.

10. Methods of Analyzing a Biological Function

The present invention further provides a method of analyzing a biological (or in vivo) function of a desired protein or a gene encoding the desired protein, comprising comparing the phenotype of the chimeric non-human animal or its progeny as prepared above with that of a control animal, i.e., a chimeric non-human animal which is produced from a corresponding wild-type pluripotent cell (e.g., ES cell) and does not contain the nucleic acid sequence encoding a desired protein (i.e., structural gene, transferred gene, or second gene); and determining a difference in phenotype between them.

In this method, any trait emerging in vivo due to the gene transfer can be detected by physicochemical methods, thereby identifying a biological function of the transferred nucleic acid sequence or the protein encoded thereby. For example, blood samples are taken from chimeric non-human animals, or progeny thereof, produced from ES cells containing a nucleic acid sequence encoding a desired protein and from control chimeric non-human animals which are produced from wild-type ES cells and contain no nucleic acid sequence encoding the desired protein; and the blood samples are analyzed by blood cell counter. By comparing blood levels of leukocytes, erythrocytes, platelets or the like between the two types of chimeric non-human animals, the effect of the desired protein encoded by the transferred nucleic acid sequence on proliferation and differentiation of blood cells can be clarified. In Examples as described later, DNA encoding erythropoietin (EPO) was used as the transferred nucleic acid sequence. In this case, the significant increase in red blood cells (i.e., trait) was observed in chimeric mice.

Now, further preferable embodiments of the present invention will be described taking the system using an immunoglobulin light-chain gene as an example.

Immunoglobulin (Ig) is one of the secretory proteins produced in the largest amount in serum. For example, immunoglobulin occupies 10 to 20% of the serum protein in humans at a level of 10 to 100 mg/ml. Immunoglobulin (Ig) is produced in B cells, mainly in terminally differentiated B cells i.e. plasma cells, in a large amount. However, various factors including high transcriptional activity in the Ig locus, stability of mRNA, and function of plasma cells specialized for secretion and production of a protein, contribute to a high level of Ig expression. Furthermore, in an adult, B cells are produced in the bone marrow and migrate to the spleen, the small intestine Peyer's patch, and the systemic lymph tissues such as the lymph node with maturation. The product of the transferred gene produced under the control region of an Ig gene of the B cell is released into the blood or the lymph in the same manner as Ig and rapidly delivered throughout the body. The present invention is advantageous since a nucleic acid sequence encoding a desired protein (i.e., structural gene, transferred gene, or second gene) is expressed by use of the Ig expression system enabling high expression.

To express a transferred gene efficiently, it is desirable to introduce the transferred gene into the gene encoding Ig light chain, preferably κ-light chain. For example, 95% of mouse immunoglobulin contains κ-light chain and only one constant region gene is present there, whereas λ-light chain is present in 5% of the mouse immunoglobulin and has 4 types of different genes, any of which is employed. The heavy chain has 8 types of constant regions, i.e. μ, γ (4 types) α, δ, and ε. Considering that a transferred gene is normally inserted at a single site of the Ig gene, use of κ-chain is desirable.

The transferred nucleic acid is desirably expressed under such conditions that functional Ig light chain is produced. A chimeric non-human animal or its progeny according to the present invention preferably contains, on the genome, a gene expression unit having an immunoglobulin light-chain gene and a promoter portion of the gene in the vicinity of the gene, and further a nucleic acid sequence (or a transferred gene) encoding a desired protein downstream of the promoter portion. To modify the genome of an animal such that the promoter portion of an immunoglobulin light-chain gene is contained in the vicinity of the gene while a nucleic acid sequence (or a transferred gene) encoding a desired protein is contained downstream of the promoter, a targeting vector is provided, in which the nucleic acid sequence encoding a desired protein is inserted. As the targeting vector, CκP2 targeting vector (see Example 5) is preferably used. The targeting vector contains a promoter portion of an immunoglobulin light-chain gene in the vicinity of the gene. Downstream of the promoter portion, an appropriate restriction enzyme cleavage site is inserted for introducing the nucleic acid sequence encoding a desired protein (i.e., structural gene, transferred gene, or second gene). At the restriction enzyme cleavage site, DNA (i.e., cDNA or genomic DNA) containing from the initiation codon to the termination codon of the transferred nucleic acid is inserted. In addition, it may be preferable to arrange a translation promoting sequence like Kozak sequence upstream of the initiation codon. Furthermore, to easily identify a homologous recombinant, a drug resistant gene marker, preferably puromycin resistant gene, may be inserted previously at an inserted position of a foreign gene.

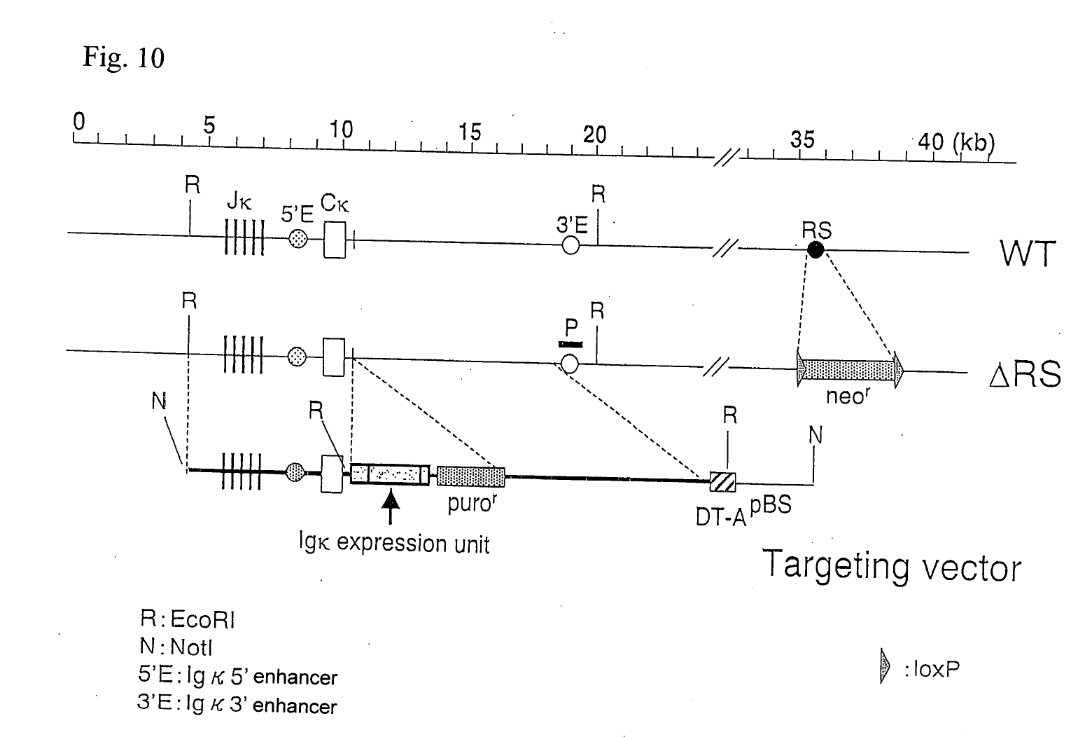

As a preferable example, use may be made of murine ES cells, more specifically ES cells having a drug resistant marker inserted into the RS element region, which is located about 25 Kb downstream of the immunoglobulin κ light chain gene, whereby the efficiency of inserting a desired gene in the vicinity of the Igκ constant region using a targeting vector can be elevated (FIG. 10).

Non-human animal ES cells can be transformed by a targeting vector in accordance with the method described by Shinich Aizawa (as above). Then, in the same manner as described in PCT international application WO 00/10383 (published Mar. 2, 2000) filed by the present applicant, puromycin resistant clones are picked up to prepare genomic DNA, which is subjected to Southern analysis to identify homologous recombinants. The puromycin resistant gene in the targeting vector is derived from the Lox-P Puro plasmid described in WO 00/10383 and includes a Lox-P sequence at both ends in a forward direction. Therefore, the puromycin resistant gene can be removed by the method as described in WO 00/10383, from the ES cell with transferred gene.

In the present invention, when an immunoglobulin light-chain gene is used, a non-human animal line homozygous for destruction of its immunoglobulin heavy chain gene (as described in WO 00/10383) is preferably used as a defective host embryo to inject an ES cell.

The prepared ES cell with transferred gene is injected into the blastocyst stage or 8 cell stage embryo from the defective host embryo by using a capillary tube. The blastocyst stage or 8 cell stage embryo is directly transplanted into the oviduct of a surrogate mother of the cognate non-human animal, or is alternatively cultured for a day up to a blastocyst embryo, which is then transplanted to the uterus of the surrogate mother. Thereafter, the surrogate mother is allowed to give birth to obtain a child animal.

In the chimeric non-human animal, matured B lymphocytes from the host embryo are not present but only those from the ES cell with transferred gene are present. This is because the non-human animal, as a host embryo, whose immunoglobulin heavy-chain has been knocked out, is devoid of matured B lymphocytes (B220 positive), whereby no immunoglobulin is detected in the blood (Tomizuka et al., Proc. Natl. Acad. Sci. USA, 97:722-727, 2000). The restoration of the production of matured B lymphocytes and antibodies in the chimeric non-human animal by contribution of the gene-transferred ES cell can be detected by the FACS analysis, ELISA, or the like. Whether the nucleic acid sequence inserted into the B cell from the knock-in ES cell is expressed depends upon whether a site-directed recombination reaction takes place in the Ig light-chain gene of the inserted allele. Thus, when recombination of the Ig light-chain gene of the inserted allele is successfully performed and mRNA encodes a functional light chain, the transferred nucleic acid (or structural gene) present concurrently on the mRNA is translated into a protein by the action of IRES. Furthermore, even when recombination of the κ-chain gene of inserted allele fails and the κ-chain or λ-chain gene of the other allele encodes a functional light chain, mRNA encoding the non-functional κ-chain and the transferred nucleic acid is transcribed, with the result that protein derived from the transferred nucleic acid can be expressed. The transferred nucleic acid is not expressed when functional recombination of the κ-chain or λ-chain gene of the other allele successfully takes place in advance and then the recombination of the Ig κ-chain of the inserted allele is shut off by the mechanism of allelic exclusion. In a non-human animal, B cells appear in the liver tissue of a fetus around day 12th of viviparity. Upon birth, the place where B-cells are developed changes to the bone marrow. In the fetus stage, the B cells remain in the initial stage of development; in other words, most of the B cells express a membrane-type immunoglobulin receptor. The number of B cells is low and the amount of mRNA encoding an immunoglobulin is low in the cells primarily expressing membrane-type Ig in the fetus compared to an adult. Based on these facts, the expression of the transferred nucleic acid in the fetus may be extremely low compared to that of the adult. Production of antibodies increases from the weaning stage (3 weeks old). This phenomenon is presumably caused by an increase of the plasma cells, terminal differentiation stage of B cells. Thereafter, B cells migrate into the lymph tissues such as the spleen, lymph node, and intestine Peyer's patch, and express antibodies and the inserted nucleic acid. Likewise, a desired protein encoded by the inserted nucleic acid is secreted into the blood and the lymph in the same manner as immunoglobulin and delivered throughout the body.

The expression of the transferred nucleic acid in the B cells can be confirmed as follows. For example, expression of mRNA by a transferred gene can be detected by RT-PCR or Northern blot using RNA derived from the tissue or cell population containing B cells, such as spleen cells and peripheral blood nucleated cells. When a specific antibody is obtained against a desired protein encoded by a transferred nucleic acid sequence, the expression of a protein can be detected by ELISA or Western blot using the chimeric mouse serum. Alternatively, if DNA encoding a transferred nucleic acid sequence is appropriately modified such that a tag peptide detectable by an antibody is added to the DNA, the expression of the transferred nucleic acid sequence can be detected by an antibody against the tag peptide, etc.

The chimeric non-human animal having a nucleic acid sequence (i.e., structural gene or second gene) encoding a desired protein efficiently introduced without fail in the aforementioned manner, highly expresses the protein. The reasons why the efficiency is high are principally based on the points described below.

(1) Since a host embryo used is devoid of B lymphocytes, B lymphocytes of a chimeric non-human animal are all derived from pluripotent cells such as the ES cells irrelevant to the chimeric rate.

(2) By virtue of use of the pluripotent cells such as the ES cells having an enhancer (+drug resistant marker) inserted into the region (for example, the RS element region about 25 Kb downstream of the κ light-chain gene), which is about 100 Kb or less, preferably 50 Kb or less, further preferably 30 Kb or less downstream of an immunoglobulin gene (for example, murine κ light-chain gene) of a non-human animal, homologous recombination takes place in the vicinity of the immunoglobulin gene at an efficiency of 30% or more, 40% or more, preferably 50% or more, and more preferably 60% or more.

(3) Expression system for immunoglobulin is used.

(4) Expression of immunoglobulin is extremely low in the initial stage of development and explosively increases after the wearing stage. For this reason, the function of a transferred gene in the adult can be investigated, even if embryonic lethal is brought by high expression of the transferred gene.

Now, the present invention will be described in detail by way of Examples, which should not be construed as limiting the scope of the present invention.

### EXAMPLES

### Example 1

Preparation of a Murine RS Element Targeting Vector, pBlueRS-LoxP-Neo-DT-A-3′KO-5′KO

(1) Preparation of KO Basic Vector, pBlueLAB-LoxP-Neo-DT-A

The following DNAs were synthesized to add new restriction sites to the vector.

| | | |
|---|---|---|
| LinkA1: | TCGAGTCGCGACACCGGCGGGCGCGCCC | (SEQ ID NO:1) |
| LinkA2: | TCGAGGGCGCGCCCGCCGGTGTCGCGAC | (SEQ ID NO:2) |
| LinkB1: | GGCCGCTTAATTAAGGCCGGCCGTCGACG | (SEQ ID NO:3) |
| LinkB2: | AATTCGTCGACGGCCGGCCTTAATTAAGC | (SEQ ID NO:4) |

Plasmid pBluescript II SK(−)(TOYOBO, Japan) was treated with restriction enzymes SalI and XhoI. The resultant reaction mixture was subjected to phenol/chloroform extraction and then to precipitation with ethanol. In order to add new restriction sites NruI, SgrAI and AscI to the plasmid, linkers, LinkA1 and LinkA2, were synthesized. The two linkers each formed of oligo nucleotide DNA were inserted into the plasmid treated with the restriction enzymes and the resultant construct was introduced into Escherichia coli DH5α. DNA was prepared from the obtained transformants. In this manner, plasmids pBlueLA were obtained.

Subsequently, the plasmid pBlueLA was treated with restriction enzymes NotI and EcoRI. The resultant reaction mixture was subjected to phenol/chloroform extraction and then to ethanol precipitation. To add new restriction sites PacI, FseI and SalI, linkers, LinkB1 and LinkB2, were synthesized. The two linkers each formed of oligo DNA were inserted into the plasmid treated with the restriction enzymes and the resultant construct was introduced into Escherichia coli DH5α. DNA was prepared from the obtained transformants. In this manner, the plasmid pBlueLAB was obtained.

The plasmid pLoxP-STneo described in WO 00/10383 (described above) was digested with XhoI to obtain a Neo resistant gene (LoxP-Neo) having a LoxP sequence at both ends. The both ends of the LoxP-Neo gene were blunt-ended with T4 DNA polymerase to obtain LoxP-Neo-B.

After the plasmid pBlueLAB was digested with EcoRV, the resultant reaction mixture was subjected to phenol/chloroform extraction and then to ethanol precipitation. After LoxP-Neo-B was inserted into the digested plasmid, the resultant product was introduced into Escherichia coli DH5α. DNA was prepared from the obtained transformants. In this manner the plasmid pBlueLAB-LoxP-Neo was obtained.

Plasmid pMC1DT-A (Lifetech Oriental, Japan) was digested with XhoI and SalI and applied to 0.8% agarose gel. About 1 kb band was resolved on the agarose gel and DT-A fragment was recovered by QIAquick Gel Extraction Kit (Qiagen, Germany) in accordance with the instructions.

After the plasmid pBlueLAB-LoxP-Neo was digested with XhoI, the resultant reaction mixture was subjected to phenol/chloroform extraction and then to ethanol precipitation. After the DT-A fragment was inserted into the plasmid, the resultant construct was introduced into Escherichia coli DH5α. DNA was prepared from the obtained transformants. In this manner, the KO basic vector pBlueLAB-LoxP-Neo-DT-A was obtained.

(2) Obtaining a 5′ Genomic Region Fragment Upstream of the Murine RS Element

Based on the genomic DNA sequence in the vicinity of the murine RS element obtained from the GenBank (NCBI, USA), the following DNA primers were synthesized.

| | | |
|---|---|---|
| RS5′ FW3: | ATAAGAATGCGGCCGCAAAGCTGGTGGGTTAAGACTATCTCGTGAAGTG | (SEQ ID NO:5) |
| RS5′ RV3: | ACGCGTCGACTCACAGGTTGGTCCCTCTCTGTGTGTGGTTGCTGT | (SEQ ID NO:6) |

A reaction mixture was prepared by use of KOD-plus- (TOYOBO, Japan) in accordance with the instructions. To the reaction mixture (50 μl), the two primers as prepared above (10 pmol each) and DNA derived from BAC clone RP23-434I4 (GenBank Accession Number: AC090291) as a template were added. After the reaction mixture was kept at 94° C. for 2 minutes and a PCR cycle consisting of 94° C. for 15 seconds and 68° C. for 5 minutes was repeated 33 times. 5 kb amplified fragment was resolved on 0.8% agarose gel. From the cut-out gel, amplified fragment was recovered by QIAquick Gel Extraction Kit (Qiagen, Germany) in accordance with the instructions. The amplified fragment thus recovered was digested with NotI and SalI and resolved on 0.8% agarose gel. From the cut-out gel, the enzyme-digested fragment was recovered by QIAquick Gel Extraction Kit (Qiagen, Germany) in accordance with the instructions.

After pBlueLAB was digested with NotI and SalI, the resultant reaction mixture was subjected to phenol/chloroform extraction and then to ethanol precipitation. The DNA fragment recovered above was inserted into the digested pBlueLAB. The resultant plasmid was inserted into Escherichia coli DH5α. From the resultant transformant, DNA was prepared and sequencing of the inserted fragment was performed. Clones having no mutation due to PCR were selected and digested with NotI and SalI to obtain fragments. Of them, the 5 kb fragment was resolved on 0.8% agarose gel. From the cut-out gel, the enzyme-digested fragment was recovered by QIAquick Gel Extraction Kit (Qiagen, Germany) in accordance with the instructions.

(3) Obtaining a 3′ Genomic Region Fragment Downstream of the Murine RS Element

The following DNA primers were synthesized based on the genomic DNA sequence in the vicinity of the murine RS element obtained from the GenBank (NCBI, USA).

| | | |
|---|---|---|
| RS3′ FW2: | TTGGCGCGCCCTCCCTAGGACTGCAGTTGAGCTCAGATTTGA | (SEQ ID NO:7) |
| RS3′ RV3: | CCGCTCGAGTCTTACTGTCTCAGCAACAATAATATAAACAGGGG | (SEQ ID NO:8) |

A reaction mixture was prepared by use of KOD-plus- (TOYOBO, Japan) in accordance with the instructions. To the reaction mixture (50 μl), the two primers as prepared above (10 pmol each) and DNA derived from BAC clone RP23-434I4 (GenBank Accession Number: AC090291) as a template were added. After the reaction mixture was kept at 94° C. for 2 minutes and a PCR cycle consisting of 94° C. for 15 seconds and 68° C. for 2 minutes was repeated 33 times. 2 kb amplified fragment was resolved on 0.8% agarose gel. From the cut-out gel, the amplified fragment was recovered by QIAquick Gel Extraction Kit (Qiagen, Germany) in accordance with the instructions. The amplified fragment thus recovered was digested with AscI and XhoI and resolved on 0.8% agarose gel. From the cut-out gel, the enzyme-digested fragment was recovered by QIAquick Gel Extraction Kit (Qiagen, Germany) in accordance with the instructions.

After pBlueLAB was digested with AscI and XhoI, the resultant reaction mixture was subjected to phenol/chloroform extraction and then to ethanol precipitation. The DNA fragment recovered above was inserted into the plasmid pBlueLAB, and the resultant plasmid was inserted into Escherichia coli DH5α. From the resultant transformant, DNA was prepared and sequencing of the inserted fragment was performed. Clones having no mutation due to PCR were selected and digested with AscI and XhoI. The obtained 2 kb fragment was resolved on 0.8% agarose gel. From the cut-out gel, the enzyme-digested fragment were recovered by QIAquick Gel Extraction Kit (Qiagen, Germany) in accordance with the instructions.

(4) Insertion of the 3′ Genomic Region Fragment Downstream of the Murine RS Element into the Basic Vector

Plasmids pBlueLAB-LoxP-Neo-DT-A were digested with AscI and XhoI, and the DNA fragment of about 7.6 Kb was separated and purified by 0.8% agarose gel electrophoresis. After the genome fragment prepared in (3) above was inserted into the 7.6 Kb fragment, and the resultant plasmid was introduced into Escherichia coli XL10-Gold Ultracompetent Cells (STRATAGENE, USA). From the resultant transformant, DNA was prepared and the nucleotide sequence of the ligation portion was confirmed.

(5) Insertion of the 5′ Genomic Region Fragment Upstream of the Murine RS Element into the KO Basic Vector Comprising the 3′ Genomic Region Fragment Downstream of the Murine RS Element

After the plasmid obtained in (4) above was digested with NotI and SalI, the resultant DNA fragment of 9.6 Kb was separated and purified by 0.8% agarose gel electrophoresis. After the genome fragment prepared in (2) above was inserted into the 9.6 Kb fragment, and the resultant plasmid was introduced into Escherichia coli XL10-Gold Ultracompetent Cells (STRATAGENE, USA). From the resultant transformant, DNA was prepared and the nucleotide sequence of the ligation portion was confirmed. In this manner, the murine RS element targeting vector pBlueRS-LoxP-Neo-DT-A-3′KO-5′KO was obtained.

### Example 2

Preparation of Murine RS Element Targeting Vector for Electroporation

60 μg of pBlueRS-LoxP-Neo-DT-A-3′KO-5′KO was digested with NotI at 37° C. for 5 hours, by using a buffer (H buffer for restriction enzyme; Roche Diagnostics, Germany) supplemented with spermidine (1 mM pH7.0; Sigma, USA). After extraction with phenol/chloroform, 2.5 volumes of 100% ethanol and 0.1 volumes of 3M sodium acetate were added to the resultant mixture and stored at −20° C. for 16 hours. The vector linearized into single stand with NotI was centrifugally collected and sterilized by adding 70% ethanol. Then, 70% ethanol was removed in a clean ventilator and the resultant product was air-dried for one hour. To the dried product, HBS solution was added to prepare a 0.5 μg/μl DNA solution and stored at room temperature for one hour. In this way, the murine RS element targeting vector pBlueRS-LoxP-Neo-DT-A-3′KO-5′KO-NotI (FIG. 1) for electroporation was prepared.

### Example 3

Preparation of a Probe for Southern Analysis of the Genome

The following DNA primers were synthesized to obtain oligo DNA containing a 573-mer region upstream of the 5′ KO based on the nucleotide sequence information of BAC clone RP23-434I4 (GenBank Accession Number: AC090291).

| | |
|---|---|
| RS5′ Southern FW1: | |
| CATACAAACAGATACACACATATAC | (SEQ ID NO:9) |
| R55′ Southern RV2: | |
| GTCATTAATGGAAGGAAGCTCTCTA | (SEQ ID NO:10) |

A reaction mixture was prepared using Takara Z Taq (Takara Shuzo, Japan) in accordance with the instructions. To the reaction mixture (50 μl), the two primers as prepared above (10 pmol each) and DNA derived from BAC clone RP23-434I as a template were added. After the reaction mixture was kept at 94° C. for 2 minutes, a PCR cycle consisting of 94° C. for 30 seconds, 60° C. for 20 seconds, and 72° C. for 1 minute was repeated 25 times. The amplified fragment of 573 mer was resolved on 0.8% agarose gel. From the cut-out gel, a probe 5′ KO-prob, for Southern analysis of the 5′-side genome, was recovered by QIAquick Gel Extraction Kit (Qiagen, Germany) in accordance with the instructions.

Based on the nucleotide sequence information of BAC clone RP23-434I4 (GenBank Accession Number: AC090291), the following DNAs were synthesized to obtain oligo DNA containing 600 mer region downstream of 3′ KO.

| | |
|---|---|
| RS3′ Southern FW1: | |
| TCTTACTAGAGTTCTCACTAGCTCT | (SEQ ID NO:11) |
| RS3′ Southern RV2: | |
| GGAACCAAAGAATGAGGAAGCTGTT | (SEQ ID NO:12) |

A reaction mixture was prepared by use of Takara Z Taq (Takara Shuzo, Japan) in accordance with the instructions. To the reaction mixture (50 μl), the two primers as prepared above (10 pmol each) and DNA derived from BAC clone RP23-434I as a template were added. After the reaction mixture was kept at 94° C. for 2 minutes, a PCR cycle consisting of 94° C. for 30 seconds, 60° C. for 20 seconds and 72° C. for 1 minute was repeated 25 times. The amplified fragment of 600 mer was resolved on 0.8% agarose gel. From the cut-out gel, a probe, 3′ KO prob, for Southern analysis of the 3′ genome side was recovered by QIAquick Gel Extraction Kit (Qiagen, Germany) in accordance with the instructions.

### Example 4

Obtaining RS Element Targeting Murine ES Cell

To obtain RS element targeting murine ES cells in a homologous recombination manner, the pBlueRS-LoxP-Neo-DT-A-3′KO-5′KO prepared in Example 2 was linearized with restriction enzyme NotI (Takara Shuzo, Japan) and introduced into murine ES cell TT2F (Yagi et al., Analytical Biochem., 214:70, 1993) in accordance with the established method (Shinichi Aizawa, Gene Targeting, in Bio-Manual Series 8, 1995, Yodosha, Japan).

TT2F cells were cultured in accordance with the method (Shinichi Aizawa, ibid) using, as a trophocyte, the G418 resistant cultured primary cell (Invitrogen, USA), which was treated with mitomycin C (Sigma, USA). The TT2F cells grown were treated with trypsin and suspended in HBS at 3×10⁷ cells/ml. Thereafter, 0.5 ml of the cell suspension was mixed with 10 μg of vector DNA, placed in a gene pulsar cuvette (distance between electrodes: 0.4 cm; Biorad, USA) and subjected to electroporation (capacity: 960 μF, voltage: 240 V, room temperature). After electroporation, the cells were suspended in 10 ml of ES medium and seeded on a 100 mm plastic tissue-culture Petri dish (Falcon; Becton, Dickinson, USA) having feeder cells previously seeded therein. After 24 hours, the medium was replaced with fresh ES medium containing 200 μg/ml neomycin (Sigma, USA). The colonies generated after 7 days were picked up, individually transferred to 24-well plates, and grown up to the confluent state. Two thirds of the grown cells were suspended in 0.2 ml of a stock medium (ES medium+10% DMSO; Sigma, USA) and stored at −80° C. The remaining one thirds was seeded on a 12-well gelatin coated plate and cultured for 2 days. From 10⁶ to 10⁷ cells, genomic DNA was prepared by use of Puregene DNA Isolation Kits (Gentra System, USA).

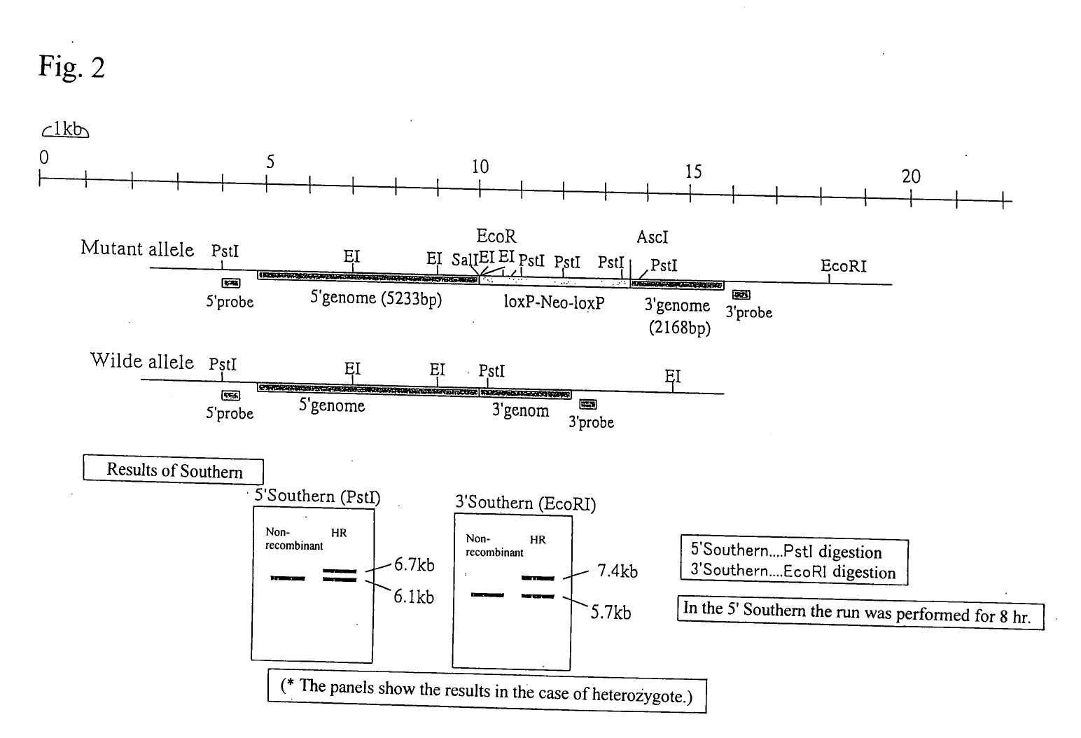

The genomic DNA of the neomycin-resistant TT2F cells was digested with restriction enzyme EcoRI (Takara Shuzo, Japan) and separated by 0.8% agarose gel electrophoresis. Subsequently, Southern blot was performed by use of, as a probe, a DNA fragment (3 ′KO-prob, see Example 3, FIG. 2), which was located downstream of the 3′ homologous region of the targeting vector, to detect homologous recombinants. In the wild-type TT2F cell, a single band (about 5.7 Kb) was detected by EcoRI digestion. In the homologous recombinant, detection of two bands (about 5.7 Kb and about 7.4 Kb) was expected. Actually, a new band of about 7.4 Kb was detected in the neomycin resistant cell line. The genomic DNA of clones which were confirmed as homologous recombinants by Southern analysis using 3′KO-prob was further digested with restriction enzyme PstI (Takara Shuzo, Japan) and separated by 0.8% agarose gel electrophoresis. Subsequently, Southern analysis was performed by use of, as a probe, a DNA fragment (5 ′KO-probe, see Example 3, FIG. 2), which is located upstream of the 5′ homologous region of the targeting vector, to detect homologous recombinants. In the wild-type TT2F cell, a single band (about 6.1 Kb) was detected by PstI digestion. In the homologous recombinant, detection of two bands (about 6.7 Kb and about 6.1 Kb) was expected. Actually, a new band of about 6.7 Kb was detected in the neomycin resistant cell line. These clones were devoid of a region of and in the vicinity of the chromosome containing the murine RS element, and instead, contained a neomycin resistant gene (comprising SV40 enhancer and restriction sites from the targeting vector at both ends). Southern analysis was performed by use of 3′ KO-prob and 5′KO-prob. As a result, when pBlueRS-LoxP-Neo-DT-A-3′KO-5′KO was linearized by restriction enzyme NotI, 9 out of 72 cell lines (12.5%) were recombinants.

The RS element targeting murine ES cell obtained was analyzed for nucleotype in accordance with the method as described by Shinichi Aizawa (ibid). As a result, it was confirmed that no abnormal nucleotype was found in the ES cells.

### Example 5

Preparation of CκP2 Targeting Vector

(1) Preparation of a Fragment in the Vicinity of a Cloning Site

A genome fragment was prepared in which a mouse immunoglobulin κ-chain promoter (P2 promoter), restriction enzyme recognition sequences (SalI, FseI and NheI recognition sequences), a mouse immunoglobulin κ chain Poly A signal region, and a puromycin resistant gene expression unit, were introduced in order at a site downstream of the mouse immunoglobulin κ chain (Igκ) constant region gene. The method will be described more specifically below.

(1.1) Preparation of a Fragment Upstream of a Cloning Site

The following DNAs were synthesized based on the gene sequence of mouse IgGκ obtained from the GenBank (NCBI, USA).

| | | |
|---|---|---|
| igkc1: | atctcgaggaaccactttcctgaggacacagtgatagg | (SEQ ID NO:13) |
| igkc2: | atgaattcctaacactcattcctgttgaagctcttgac | (SEQ ID NO:14) |

An XhoI recognition sequence was added to the end of 5′ primer igkc1, while an EcoRI recognition sequence to the end of 3′ primer igkc2. A reaction mixture was prepared in accordance with the instructions attached to Takara LA-Taq (Takara Shuzo, Japan). To the reaction mixture (50 μl), the two primers as prepared above (10 pmol each) and, as a template, 25 ng of pBluescript SKII (+) (TOYOBO, Japan) into which a DNA fragment derived from λ clone containing Ig light chain Cκ-Jκ had been cloned (WO 00/10383), were added. After the reaction mixture was kept at 94° C. for 1 minute, a PCR cycle consisting of 94° C. for 30 seconds and 68° C. for 3 minutes was repeated 25 times. The obtained reaction mixture was subjected to phenol/chloroform extraction, ethanol precipitation, digestion with EcoRI and XhoI, and subjected to 0.8% agarose gel electrophoresis to resolve the DNA fragment on the gel. Desired DNA fragment was recovered by Gene Clean II (Bio 101, USA) to obtain amplified fragment A. After the vector pBluescript II KS− (Stratagene, USA) was digested with EcoRI and XhoI, the ends of the vector were dephosphorylated with E. coli alkaline phosphatase. Into the resultant vector was inserted the amplified fragment A, and then the product was introduced into Escherichia coli DH5° C. DNA was prepared from the obtained transformant and the nucleotide sequence was confirmed. In this manner, the plasmid pIgCκA was obtained.

(1.2) Preparation of a Fragment Downstream of the Cloning Site

The following DNAs were synthesized based on the mouse IgGκ gene sequence obtained from the GenBank (NCBI, USA).

| | | |
|---|---|---|
| igkc3: | atgaattcagacaaaggtcctgagacgccacc | (SEQ ID NO:15) |
| igkc4: | atggatcctcgagtcgactggatttcagggcaactaaacatt | (SEQ ID NO:16) |

An EcoRI recognition sequence was added to the end of 5′ primer igkc3, while BamHI, XhoI and SalI recognition sequences were added to the end of 3′ primer igkc4 in order from the 5′ side. A reaction mixture was prepared in accordance with the instructions attached to Takara LA-Taq (Takara Shuzo, Japan). To the reaction mixture (50 μl), the two primers as prepared above (10 pmol each) and, as a template, 25 ng of pBluescript SKII (+) (TOYOBO, Japan) into which a DNA fragment derived from λ clone containing Ig light chain Cκ-Jκ had been cloned (WO 00/10383), were added. After the reaction mixture was kept at 94° C. for 1 minute, a PCR cycle consisting of 94° C. for 30 seconds, 55° C. for 30 seconds and 72° C. for 1 minute was repeated 25 times. The obtained reaction mixture was subjected to phenol/chloroform extraction, ethanol precipitation, digestion with EcoRI and BamHI, and subjected to 0.8% agarose gel electrophoresis to resolve the DNA fragment on the gel. Desired DNA fragment was recovered by use of Gene Clean II (Bio 101, USA) to obtain amplified fragment B. After the vector pIgCκA was digested with EcoRI and BamHI, the ends of the vector were dephosphorylated with E. coli alkaline phosphatase. Into the resultant pIgCκA vector was inserted the amplified fragment B, and then the product was introduced into Escherichia coli DH5α. DNA was prepared from the obtained transformant and the nucleotide sequence was confirmed. In this manner, plasmid pIgCκAB was obtained.

(2) Introduction of Puromycin Resistant Gene

Lox-P Puro plasmid (WO 00/10383) was digested with EcoRI and XhoI and blunt-ended with T4DNA polymerase. DNA fragments were separated by 0.8% agarose gel electrophoresis. The DNA fragment containing the IoxP-puromycin resistant gene was recovered by use of Gene Clean II (Bio 101, USA). Plasmid pIgCκAB was digested with SalI and blunt-ended. Into the blunt-ended plasmid was inserted the obtained loxP-puromycin resistant gene fragment, and then the plasmid was introduced into Escherichia coli DH5α. DNA was prepared from the obtained transformant and the nucleotide sequence of the ligation portion was confirmed. In this manner, plasmid pIgCκABP was obtained.

(3) Introduction of IRES Gene

The following DNAs were synthesized based on the IRES gene sequence derived from encephalomyocarditis virus (available from the GenBank (NCBI, USA)).

| | | |
|---|---|---|
| eIRESFW: | atgaattcgcccctctccctccccccccccta | (SEQ ID NO:17) |
| esIRESRV: | atgaattcgtcgacttgtggcaagcttatcatcgtgtt | (SEQ ID NO:18) |

An EcoRI recognition sequence was added to the end of 5′ primer eIRESFW, while EcoRI and SalI recognition sequences were added to the end of 3′ primer esIRESRV in order from the 5′ side. A reaction mixture was prepared in accordance with the instructions attached to Takara LA-Taq (Takara Shuzo, Japan). To the reaction mixture (50 μl), the two primers as prepared above (10 pmol each) and, as a template, 150 ng of pIREShyg plasmid (Clontech, USA) were added. After the reaction mixture was kept at 94° C. for 1 minute and a PCR cycle consisting of 94° C. for 30 seconds, 55° C. for 30 seconds and 72° C. for 1 minute was repeated 25 times. The obtained reaction mixture was subjected to 0.8% agarose gel electrophoresis to separate DNA fragments. Desired DNA fragment was recovered by use of Gene Clean II (Bio 101, USA). The obtained DNA fragment was inserted into pGEM-T vector (Promega, USA) and then introduced into Escherichia coli DH5α. DNA was prepared from the obtained transformant and the nucleotide sequence was confirmed. In this manner, plasmid IRES-Sal/pGEM were obtained. The plasmid was digested with EcoRI and subjected to 0.8% agarose gel electrophoresis to separate DNA fragments. Desired DNA fragment was obtained by use of Gene Clean II (Bio 101, USA). The obtained IRES gene was inserted into the pIgCκ ABP plasmid digested with EcoRI and the resultant plasmid was introduced into Escherichia coli DH5α. DNA was prepared from the obtained transformant, the nucleotide sequence of the ligated portion was confirmed. In this manner, plasmid pIgCκABPIRES was obtained.

(4) Preparation of Plasmid pΔCκSal

Targeting vector plasmid for targeting the immunoglobulin gene κ-light chain described in WO 00/10383 was digested with SacII and thereafter was partially digested with EcoRI. The LoxP-PGKPuro portion was cut out after 0.8% agarose gel electrophoresis and the remaining 14.6 kb DNA was separated from the gel and recovered by use of Gene Clean II (Bio 101, USA). Into the obtained DNA were inserted the following synthesized DNAs. In this manner a SalI recognition sequence was introduced.

| | | |
|---|---|---|
| Sal1 plus: | agtcgaca | |
| Sal1 minus: | aatttgtcgactgc | (SEQ ID NO:19) |

The obtained plasmid was introduced into Escherichia coli DH5α. DNA was prepared from the obtained transformant. In this manner, plasmid pΔCκSal was obtained.

(5) Preparation of Plasmid pKIκ

The pIgCκ ABPIRES plasmid obtained in (3) above was digested with XhoI. The DNA fragments were separated by 0.8% agarose gel electrophoresis. The DNA fragment containing Cκ-IRES-loxP-puromycin resistant gene was recovered by use of Gene Clean II (Bio 101, USA). After pCκSal plasmid prepared in (2) above was digested with SalI, the ends of the plasmid were dephosphorylated with E. coli alkaline phosphatase. Into the resultant plasmid was inserted the DNA fragment, and then the product was introduced into Escherichia coli DH5α. DNA was prepared from the obtained transformant and nucleotide sequence of the ligation portion was confirmed. In this manner, plasmid pKIκ was obtained.

(6) Preparation of CκΔIRES Fragment

The plasmid pIgCκ ABPIRES obtained in (3) above was partially digested with EcoRI and BgIII. The DNA fragments were separated by 0.8% agarose gel electrophoresis. The DNA fragment (i.e., IgCκΔIRES fragment), from which the IRES portion had been removed, was recovered by use of Gene Clean II (Bio 101, USA).

(7) Preparation of P2 Promoter Fragment

The following DNAs were synthesized based on the gene sequence of the mouse Igκ promoter region obtained from the GenBank (NCBI, USA).

| | | |
|---|---|---|
| P2F: | CCCAAGCTTTGGTGATTATTCAGAGTAGTTTTAGATGAGTGCAT | (SEQ ID NO:20) |
| P2R: | ACGCGTCGACTTTGTCTTTGAACTTTGGTCCCTAGCTAATTACTA | (SEQ ID NO:21) |

A HindIII recognition sequence was added to the 5′ primer P2F, and SalI recognition sequence was added to the 3′ primer P2R. The DNA fragment amplified with KOD plus (TOYOBO, Japan) using a mouse genome DNA as a template was extracted with phenol/chloroform and recovered by ethanol precipitation. The DNA fragment thus recovered was digested with HindIII and SalI and separated by 0.8% agarose gel electrophoresis. Desired DNA fragment was recovered by use of Gene Clean II (Bio 101, USA). After pBluescript IIKS-vector (Stratagene, USA) was digested with HindIII and SalI, the ends of the vector were dephosphorylated with E. coli alkaline phosphatase. Into the resultant vector was inserted the amplified fragment, and then the product was introduced into Escherichia coli DH5α. DNA was prepared from the obtained transformant, the nucleotide sequence was confirmed. In this manner, a plasmid containing an Igκ promoter region gene sequence was obtained. The obtained plasmid was digested with HindIII and SalI, DNA fragments were separated by 0.8% agarose gel electrophoresis, and P2 promoter fragment was recovered by use of Gene Clean II (Bio 101, USA).

(8) Preparation of Partial Length CκpolyA Fragment

The following DNAs were synthesized based on the mouse IgCκ poly A region gene sequence obtained from the GenBank (NCBI, USA).

| | | |
|---|---|---|
| PPF: | ACGCGTCGACGCGGCCGGCCGCGCTAGCAGACAAAGGTCCTGAGACGCCACCAC | (SEQ ID NO:22) |
| | CAGCTCCCC | |
| PPR: | GAAGATCTCAAGTGCAAAGACTCACTTTATTGAATATTTTCTG | (SEQ ID NO:23) |

SalI, FseI and NheI recognition sequences were added to the 5′ primer PPF, while BglII recognition sequence to the 3′ primer PPR. DNA fragment amplified by KOD plus (TOYOBO, Japan) using the murine genomic DNA as a template was recovered by phenol/chloroform extraction and ethanol precipitation. The DNA fragment thus recovered was digested with SalI and BglII and separated by 0.8% agarose gel electrophoresis. Desired DNA fragment was recovered by Gene Clean II (Bio 101, USA). After pSP72 vector (Promega, USA) was digested with SalI and BglII, the ends of the vector were dephosphorylated with E. coli alkaline phosphatase. Into the resultant vector was inserted the recovered fragment, and then the product was introduced into Escherichia coli DH5α. DNA was prepared from the obtained transformant and the nucleotide sequence was confirmed. In this manner, a plasmid containing partial CκpolyA region gene sequence was obtained. After the obtained plasmid was digested with SalI and BglII, DNA fragment was separated by 0.8% agarose gel electrophoresis and recovered by Gene Cleans II (Bio101, USA). In this manner, the partial length CκpolyA fragment was recovered.

(9) Preparation of a Full-Length CκpolyA Fragment

The following DNAs were synthesized based on the mouse IgCκ poly A region gene sequence obtained from the GenBank (NCBI, USA).

| | | |
|---|---|---|
| TPF: | GGAATTCAGACAAAGGTCCTGAGACGCCACCACCAGCTCCCC | (SEQ ID NO:24) |
| TPR: | CCCAAGCTTGCCTCCTCAAACCTACCATGGCCCAGAGAAATAAG | (SEQ ID NO:25) |

An EcoRI recognition sequence was added to the 5′ primer TPF, while HindIII recognition sequence to the 3′ primer TPR. DNA fragment amplified by KOD plus (TOYOBO, Japan) using the murine genomic DNA as a template was recovered by phenol/chloroform extraction and ethanol precipitation. The DNA fragment thus recovered was digested with EcoRI and HindIII and separated by 0.8% agarose gel electrophoresis. Desired DNA fragment was recovered by Gene Clean II (Bio101, USA). After pBluescript IIKS− vector (Stratagene, USA) was digested with EcoRI and HindIII, the ends of the vector were dephosphorylated with E. coli alkaline phosphatase. Into the resultant vector was inserted the recovered and amplified fragment, and then the product was introduced into Escherichia coli DH5α. DNA was prepared from the obtained transformant and the nucleotide sequence was confirmed. In this manner, a plasmid containing full-length CκpolyA region gene sequence was obtained. The obtained plasmid was digested with EcoRI and HindIII and DNA fragments were separated by 0.8% agarose gel electrophoresis. A desired DNA fragment was recovered by Gene Clean II (Bio101, USA). In this manner, the full-length CκpolyA fragment was recovered.

(10) Preparation of DNA Fragment A Consisting of Full-Length CκpolyA Fragment, P2 Promoter Fragment, and Partial Length CκpolyA Fragment

After pBluescript IIKS− vector (Stratagene, USA) was digested with EcoRI and BglII, the ends of the vector were dephosphorylated with E. coli alkaline phosphatase. Into the resultant vector were inserted the full-length CκpolyA fragment, the P2 promoter fragment, and the partial length CκpolyA fragment, and then the product was introduced into Escherichia coli DH5α. DNA was prepared from the obtained transformant, and it was confirmed at nucleotide level that the full-length CκpolyA fragment, P2 promoter fragment, and partial length CκpolyA fragment were inserted in order. In this manner, the plasmid containing DNA fragment A gene sequence was obtained. After the obtained plasmid was digested with EcoRI and BglII, DNA fragments were separated by 0.8% agarose gel electrophoresis. DNA fragment A was recovered by Gene Clean II (Bio101, USA).

(11) Preparation of pIgCκΔIRES ProA Plasmid

Into pIgCκΔIRES fragment whose ends had been dephosphorylated with E coli alkaline phosphatase, DNA fragment A was inserted. The resultant plasmid was introduced into Escherichia coli DH5α. DNA was prepared from the obtained transformant. Whether DNA fragment A was introduced was confirmed at nucleotide level. In this manner, the pIgCκΔIRES ProA plasmid containing DNA fragment A gene sequence was obtained.

(12) Preparation of Plasmid CκP2H

After pIgCκΔIRES ProA plasmid was digested with XhoI, DNA fragments were separated by 0.8% agarose gel electrophoresis. A DNA fragment constituted of the genomic region upstream of IgCκ, IgCκ, DNA fragment A, and Lox-P Puro fragment was recovered. After plasmid pΔCκSalI was digested with SalI, the ends of the plasmid were dephosphorylated with E. coli alkaline phosphatase. Into the pΔCκSalI plasmid was inserted the recovered DNA fragment, and then the product was introduced into Escherichia coli XL10-GOLD (Stratagene, USA). DNA was prepared from the obtained transformant. Whether the DNA fragment had been constituted of the genomic region upstream of IgCκ, IgCκ, DNA fragment A, and Lox-P Puro fragment was determined at nucleotide level. In this manner, the CκP2H plasmid was obtained.

(13) Preparation of Cκ5′ Genomic Plasmid

The following DNAs were synthesized based on the gene sequence of the mouse IgCκ obtained from the GenBank (NCBI, USA) and the upstream genomic region gene sequence.

| | | |
|---|---|---|
| 5GF: | ATAAGAATGCGGCCGCCTCAGAGCAAATGGGTTCTACAGGCCTAACAACCT | (SEQ ID NO:26) |
| 5GR: | CCGGAATTCCTAACACTCATTCCTGTTGAAGCTCTTGACAATGG | (SEQ ID NO:27) |

A NotI recognition sequence was added to the 5′ primer 5GF, while an EcoRI recognition sequence to the 3′ primer 5GR. DNA fragments amplified by KOD plus (TOYOBO, Japan) using the murine genomic DNA as a template were recovered by phenol/chloroform extraction and ethanol precipitation. The DNA fragment thus recovered was digested with NotI and EcoRI and separated by 0.8% agarose gel electrophoresis. Desired DNA fragment was recovered by Gene Clean II (Bio101, USA). After pBluescript IIKS− vector (Stratagene, USA) was digested with NotI and EcoRI, the ends of the vector were dephosphorylated with E. coli alkaline phosphatase. Into the resultant vector was inserted the recovered and amplified fragment, and then the product was introduced into Escherichia coli DH5α. DNA was prepared from the obtained transformant and the nucleotide sequence was confirmed. In this manner, the Cκ5′ genomic plasmid containing the Cκ5′ genomic region gene sequence was obtained.

(14) Preparation of Plasmid CκP2KIΔDT After CκP2H plasmid was digested with EcoRI and XhoI, 11 Kb DNA fragment was separated by 0.8% agarose gel electrophoresis. DNA fragment having an EcoRI site at the 5′ end and an XhoI site at the 3′ end was recovered by Gene Clean II (Bio101, USA). After Cκ5′ genomic plasmid was digested with EcoRI and XhoI, the ends of the plasmid was dephosphorylated with E. coli alkaline phosphatase. Into the resultant plasmid was inserted the DNA fragment, and then the product was introduced into Escherichia coli XL10-GOLD (Stratagene, USA). DNA was prepared from the obtained transformant. Whether the recovered fragment was inserted into the Cκ5′ genomic plasmid was determined at nucleotide level. In this manner, the plasmid CκP2KIκDT was obtained.

(15) Preparation of DT-A Fragment

After pKIκ plasmid was digested with XhoI and KpnI, DNA fragment of about 1 Kb was separated by 0.8% agarose gel electrophoresis and then DT-A fragment was obtained by use of Gene clean II (Bio101, USA).

(16) Preparation of CκP2 Targeting Vector

After plasmid CκP2KIΔDT was digested with XhoI and KpnI, the ends of the plasmid were dephosphorylated with E. coli alkaline phosphatase. Into the resultant plasmid was inserted the DT-A fragment and then the product was introduced into Escherichia coli XL10-GOLD (Stratagene, USA). DNA was prepared from the obtained transformant. Whether the DT-A fragment was inserted into the plasmid CκP2KIΔDT was determined at nucleotide level. In this manner, the CκP2 targeting vector was obtained (FIG. 3).

### Example 6

Insertion of Human EPO Gene into CκP2 Targeting Vector

(1) Preparation of Human Erythropoietin DNA Fragment

| | | |
|---|---|---|
| hEPO Np: | CCGCTCGAGCGGCCACCATGGGGGTGCACGAATGTCCTG | (SEQ ID NO:28) |
| hEPO Rp: | CCGCTCGAGCGGTCATCTGTCCCCTGTCCTGCA | (SEQ ID NO:29) |

A reaction mixture was prepared using KOD-plus- (TOYOBO, Japan) in accordance with the instructions. To the reaction mixture (50 μl), the two primers as prepared above (10 pmol each) and human EPO cDNA as a template were added. After the reaction mixture was kept at 94° C. for 2 minutes, a PCR cycle consisting of 94° C. for 15 seconds and 68° C. for 1 minute was repeated 30 times. 580 bp amplified fragment was resolved on 0.8% agarose gel. From the cut-out gel, the amplified fragment was recovered by QIAquick Gel Extraction Kit (Qiagen, Germany) in accordance with the instructions. The amplified fragment thus recovered was digested with XhoI and resolved on 0.8% agarose gel. From the cut-out gel, the enzyme-digested fragment was recovered by QIAquick Gel Extraction Kit (Qiagen, Germany) in accordance with the instructions.

After pBluescript IISK(−)(STRATAGENE, USA) was digested with XhoI, and separated and purified by 0.8% agarose gel electrophoresis, the ends of the plasmid were dephosphorylated by alkaline phosphatase from the fetal bovine intestine. Into the resultant plasmid was inserted the DNA fragment as recovered above, and the product was then introduced into Escherichia coli DH5α. DNA was prepared from the obtained transformant, and the inserted fragment was sequenced. A clone having no mutation due to PCR was selected, digested with XhoI, and resolved on 0.8% agarose gel. From the cut-out gel, the human Erythropoietin DNA fragment was recovered by QIAquick Gel Extraction Kit (Qiagen, Germany) in accordance with the instructions.

(2) Construction of Human EPO Targeting Vector

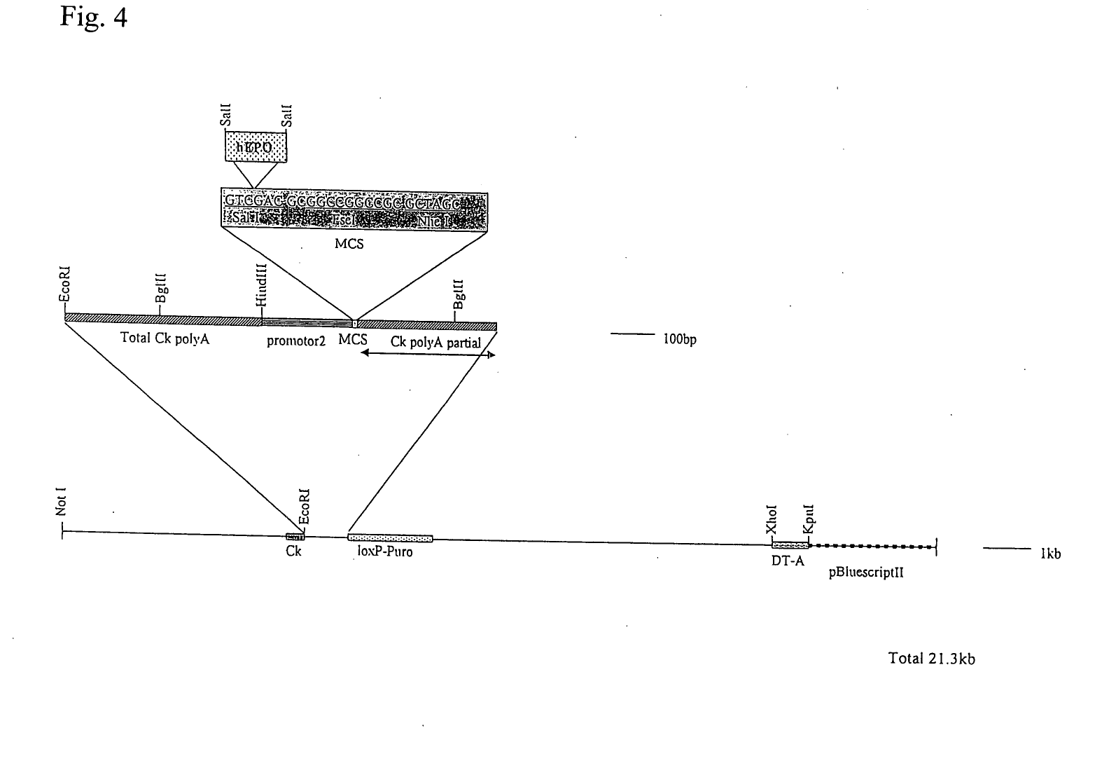

After CκP2 targeting vector was digested with SalI and the ends of the vector were dephosphorylated with alkaline phosphatase from the fetal bovine intestine. Into the resultant vector was inserted the human Erythropoietin DNA fragment as prepared in (1) above and then the product was introduced into Escherichia coli XL10-Gold Ultracompetent Cells (STRATAGENE, USA). DNA was prepared from the obtained transformant and the nucleotide sequence of the ligated portion was confirmed. In this manner, the human EPO targeting vector was obtained (FIG. 4).

### Example 7

Preparation of Human EPO Targeting Vector for Electroporation

60 μg of human EPO targeting vector was digested with XhoI at 37° C. for 5 hours by using a buffer (H buffer for restriction enzyme; Roche Diagnostics, Germany) supplemented with spermidine (1 mM pH7.0; Sigma, USA). After extraction with phenol/chloroform, 2.5 volumes of 100% ethanol and 0.1 volumes of 3M sodium acetate were added to the resultant mixture and stored at −20° C. for 16 hours. The vector which had been linearized into single stand with NotI was centrifugally collected and sterilized by adding 70% ethanol. Then, 70% ethanol was removed in a clean ventilator and the linearized vector was air-dried for one hour. To the dried vector was added HBS solution, thereby preparing a 0.5 μg/μl DNA solution, and the obtained DNA solution was stored at room temperature for one hour. In this manner, the EPO targeting vector for electroporation was prepared.

### Example 8

Obtaining ES Cell Line with Human EPO Gene Transferred

Murine ES cell can generally be established as mentioned below. Male and female mice were crossed. After fertilization, the embryo of 2.5 days old was taken and cultured in vitro in a medium for ES cell (ES medium). The embryo was allowed to develop into the blastocyst stage and separated, and subsequently seeded on the feeder-cell culture medium and cultured. Then, the cell mass which grew in a form like ES from was dispersed in the ES medium containing trypsin, cultured in a feeder-cell medium, and further sub-cultured in the ES medium. The grown cell was isolated.

To obtain a murine ES cell line with human EPO-cDNA inserted downstream of the immunoglobulin κ light-chain gene by homologous recombination, the human EPO targeting vector as prepared in Example 6 was linearized with restriction enzyme NotI (Takara Shuzo, Japan) and introduced into the murine ES cell line TT2F (Yagi et al., Analytical Biochemistry, 214:70, 1993) in accordance with the established method of Shinichi Aizawa (ibid).

The murine ES cell was cultured in accordance with the method of Shinichi Aizawa (ibid) using, as a trophocyte, the G418 resistant primary cultured cell (Invitrogen, USA) which had been treated with mitomycin C (Sigma, USA). The TT2F cells grown were treated with trypsin and suspended in HBS at 3×10⁷ cells/ml. Thereafter, 0.5 ml of the cell suspension was mixed with 10 μg of vector DNA, placed in a gene pulsar cuvette (distance between electrodes: 0.4 cm; Biorad, USA), and subjected to electroporation (capacity: 960 μF, voltage: 240 V, room temperature). After electroporation, the cells were suspended in 10 ml of ES medium (Shinichi Aizawa, ibid) and seeded on a 100 mm plastic tissue-culture Petri dish (Falcon; Becton Dickinson, USA) having feeder cells previously seeded therein. After 36 hours, the medium was replaced with fresh ES medium containing 0.8 μg/ml puromycin (Sigma, USA). After 7 days, colonies generated. Of them, 89 colonies were picked up and grown up to the confluent state in 24-well plates. Two thirds of the grown cells were suspended in 0.2 ml of a stock medium (ES medium+10% DMSO; Sigma, USA) and stored at −80° C. The remaining one thirds was seeded on a 12-well gelatin coated plate and cultured for 2 days. From 10⁶ to 10⁷ cells, genomic DNA was prepared by use of Puregene DNA Isolation Kits (Gentra System, USA).

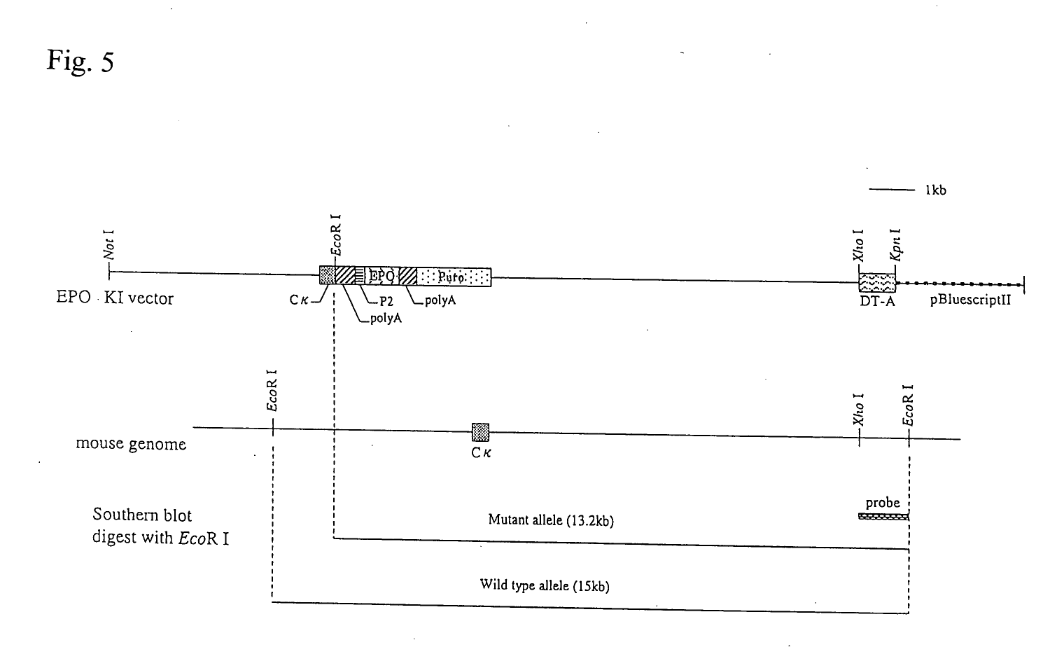

The genomic DNA from the puromycin-resistant murine ES cells was digested with restriction enzyme EcoRI (Takara Shuzo, Japan) and separated by agarose gel electrophoresis. Subsequently, Southern blot was performed by use of, as a probe, a DNA fragment (XhoI to EcoRI, about 1.4 kb, FIG. 5), which was at the 3′ end of the Ig light chain Jκ-Cκ genomic DNA and had been used in the invention described in WO 00/10383 (see Example 48), to detect homologous recombinants. As a result, 15 homologous recombinants (16.9%) were obtained out of 89 clones. In the wild-type TT2F cell, a single band was detected by EcoRI digestion. In the homologous recombinants, a new band was expected to appear below this band (WO 00/10383, see Example 58). Actually, the new band was detected in the puromycin resistant cell line. In short, these clones had human EPO-cDNA inserted downstream of the immunoglobulin κ-light-chain gene of one of the alleles.

### Example 9

Obtaining the ES Cell Line Having the Human EPO Gene Introduced Therein by RS Element Targeting Murine ES Cell Line

To obtain the murine ES cell line having human EPO-cDNA inserted downstream of the immunoglobulin κ light-chain gene by homologous recombination, the human EPO targeting vectors as prepared in Example 7 was linearized by restriction enzyme NotI (Takara Shuzo., Japan) and introduced into the RS element targeting murine ES cell in accordance with the established method (Shinichi Aizawa, ibid).

The RS element targeting murine ES cells were cultured in accordance with the method (Shinichi Aizawa, ibid) using, as a trophocyte, the G418 resistant primary cultured cell (Invitrogen, USA) treated with mitomycin C (Sigma, USA). The TT2F cells grown were treated with trypsin and suspended in HBS at 3×10⁷ cells/ml. Thereafter, 0.5 ml of the cell suspension was mixed with 10 μg of vector DNA, placed in a gene pulsar cuvette (distance between electrodes: 0.4 cm; Biorad, USA) and subjected to electroporation (capacity: 960 μF, voltage: 240 V, room temperature). After electroporation, the cells were suspended in 10 ml of the ES medium (Shinichi Aizawa, ibid) and seeded on a 100 mm plastic tissue-culture Petri dish (Falcon; Becton Dickinson, USA) having feeder cells previously seeded therein. After 36 hours, the medium was replaced with fresh ES medium containing 0.8 μg/ml puromycin (Sigma, USA). After 7 days, colonies generated. Of them, 24 colonies were picked up, individually transferred to 24-well plates, and grown up to the confluent state. Two thirds of the grown cells were suspended in 0.2 ml of a stock medium (ES medium+10% DMSO; Sigma, USA) and stored at −80° C. The remaining one thirds was seeded on a 12-well gelatin coated plate and cultured for 2 days. From 10⁶ to 10⁷ cells, genomic DNA was prepared by use of Puregene DNA Isolation Kits (Gentra System, USA). The genomic DNA from the puromycin-resistant RS element targeting murine ES cells was digested with restriction enzyme EcoRI (Takara Shuzo, Japan) and separated by agarose gel electrophoresis. Subsequently, Southern blot was performed by use of, as a probe, a DNA fragment (XhoI to EcoRI, about 1.4 kb, FIG. 5), which was at the 3′ end of the Ig light chain JκCκ genomic DNA and had been used in the invention described in WO 00/10383 (see Example 48), to detect homologous recombinants. As a result, 15 homologous recombinants (62.5%) were obtained out of 24 clones. In the wild-type TT2F cell, a single band was detected by EcoRI digestion. In the homologous recombinants, a new band was expected to appear below this band (WO 00/10383, see Example 58). Actually, the new band was detected in the puromycin resistant cell line. In short, these clones had human EPO-cDNA inserted downstream of the immunoglobulin κ-chain gene of one of the alleles.

As is apparent from the results obtained in Examples 8 and 9, murine embryonic stem cells (ES cells) in which one allele of the RS element, which was located about 25 kb downstream of the immunoglobulin κ light chain constant region gene on the murine chromosome 6, was replaced by the neomycin resistant gene, contributed to the improved efficiency of homologous recombination using the CκP2 targeting vector.

### Example 10

Preparation of Chimeric Mouse by Using Murine ES Cell Line Having the Human EPO Gene Introduced Therein and B-Lymphocyte Defective Murine Host Embryo

A homozygote from which the immunoglobulin μ chain gene was knocked out is devoid of functional B lymphocytes and thus no antibodies are produced (Kitamura et al., Nature, 350:423-426, 1991). A male and female of such a homozygote were raised in clean environment and crossed to obtain an embryo. This embryo was used as a host in this Example for producing a chimeric mouse. In this case, most of the functional B lymphocytes of the chimeric mouse were derived from the ES cell externally injected. In this Example, a mouse from which the immunoglobulin μ chain gene was knocked out and described in the report of Tomizuka et al. (Proc. Natl. Acad. Sci. USA, 97:722-7, 2000) was back-crossed with MCH (ICR) (CLEA Japan, Japan) three or more times. From the resultant mouse individuals, a host embryo was prepared.

The puromycin resistant murine ES cell line (obtained in Example 8 (#46) or Example 9 (#30)), which was confirmed that human EPO-cDNA had been inserted downstream of the immunoglobulin κ light-chain gene, was thawed from frozen stocks. The ES cells were injected in a rate of 8-10 cells/embryo into the 8-cell embryo which was obtained by crossing the male and female homozygote mice in which the immunoglobulin μ chain gene was knocked out. The embryo was cultured in the ES medium (Shinichi Aizawa, ibid) overnight to develop into the blastocyst. About 10 embryos were transplanted in each one of the two uteri of a surrogate MCH (ICR) mouse 2.5 days after pseudopregnancy treatment was applied to the mouse. Embryos to be injected (or injection embryos) were prepared by use of ES cell #46 (Example 8). When 40 injection embryos were transplanted, 9 chimeric mice were born. Chimeric mouse indivisuals were identified by evaluating whether the wild hair color (i.e., dark brown) derived from the ES cell was observed in white hair color derived from the host embryo. As a result, 5 out of 9 mice were chimeric. The 5 mice were clearly observed to partially have the wild hair color derived from the ES cell in the white hair color. Injection embryos were prepared by using ES cell line #30 (Example 9). When 80 injection embryos were transplanted, 65 mice were born. Chimeric mice were identified by evaluating whether the wild hair color (i.e., dark brown) derived from the ES cell was observed in the white hair color derived from the host embryo. As a result, 19 out of 65 mice were chimeric. The 19 mice were clearly observed to partially have the wild hair color derived from the ES cells in the white hair color.

From these results, it was demonstrated that the puromycin resistant murine ES cell line #46 and the puromycin resistant RS element targeting murine ES cell line #30, wherein both cell lines had human EPO-cDNA inserted downstream of the immunoglobulin κ chain gene, had a chimera formation potency, or a potency differentiating into normal murine tissues.

### Example 11

Increase of Erythrocyte Counts in Chimeric Mouse Derived from ES Cell with Human EPO Gene Transferred

Blood was taken from the orbita of each of 5 chimeric mice (chimeric rate: 60 to 5%; prepared in Example 10), which were derived from the puromycin resistant murine ES cell line #46 with human EPO-cDNA inserted, and 5 non-chimeric mice when they reached 8-weeks old. Then, peripheral blood cell counts were measured by means of a blood cell counter (ADVIA 120 HEMATOLOGY SYSTEM; Bayer Medical, Japan). In the chimeric mouse group, the number of erythrocytes increased 1.59 fold (in average) as large as that in the non-chimeric mice irrelevant to the chimeric rate. Similarly, blood was taken from the orbita of each of 12 chimeric mice (chimeric rate: 100 to 5%; prepared in Example 10), which were derived from the puromycin resistant RS element targeting murine ES cell line #30 with human EPO-cDNA inserted, and 5 non-chimeric mice when they reached 8-weeks old. Then, peripheral blood cell counts were measured by means of a blood cell counter (ADVIA 120 HEMATOLOGY SYSTEM; Bayer Medical, Japan). In the chimeric mouse group, the number of erythrocytes increased 1.81 fold (in average) as large as that in the non-chimeric mice irrelevant to the chimeric rate.

Thus, the significant increase of erythrocytes was observed in the mice using the puromycin resistant murine RS element targeting murine ES cell line, demonstrating that the protein encoded by the introduced human EPO gene can control the number of erythrocytes in murine blood. In other words, even when using, as an embryonic stem cell, the murine ES cell with the neomycin-resistant gene inserted in the region in which one allele of RS element was present, the method according to the invention is also useful for analyzing the function of a gene or a gene product in vivo, as in conventional murine ES cells.

### Example 12

Removal of Neomycin-Resistant Marker Gene (Comprising SV40 Enhancer) from the RS Element Targeting Murine ES Cell Line

The RS element targeting murine ES cell line RS32 (G418: Neo resistant cell line) obtained in Example 4 was demonstrated that it had normal nucleotype and high chimera formation potency. From the RS 32 cell line, the Neo resistant marker gene (comprising SV 40 enhancer) was removed by the following procedure. Expression vector pBS185, which contains the Cre recombinase gene and can cause site-directed recombination between loxP sequences inserted onto both sides of the Neo resistance marker gene, was introduced into the RS 32 cell line in accordance with the method described by Shinichi Aizawa (ibid). The resultant RS 32 cells were treated with trypsin and suspended in HBS at 2.5×10⁷ cells/ml. To the suspension, 30 μg of pBS185 DNA was added and subjected to electroporation by using gene pulsar (Biorad, USA). More specifically, the voltage of 250 V (960 μF in capacity) was applied to a 4 mm-long electroporation cell (165-2088; Biorad, USA) containing said suspension. The cells treated by electroporation were suspended in 5 ml of ES medium and seeded on a 100-mm tissue-culture plastic Petri dish having feeder cells previously seeded therein. After 2 days, the cells were treated with trypsin and seeded again in three 100-mm Petri dishes having feeder cells in a rate of 100, 300 or 800 cells per dish, respectively. After 7 days, colonies generated. Of them, 96 colonies were picked up, treated with trypsin, divided into two portions. One of them was seeded on a 48-well plate having feeder cells previously seeded therein, while the other was seeded on a 48-well plate coated only with gelatin. The latter was cultured in a medium containing 200 μg/ml G418 for 3 days. G418 resistance was determined based on the survival of cells.

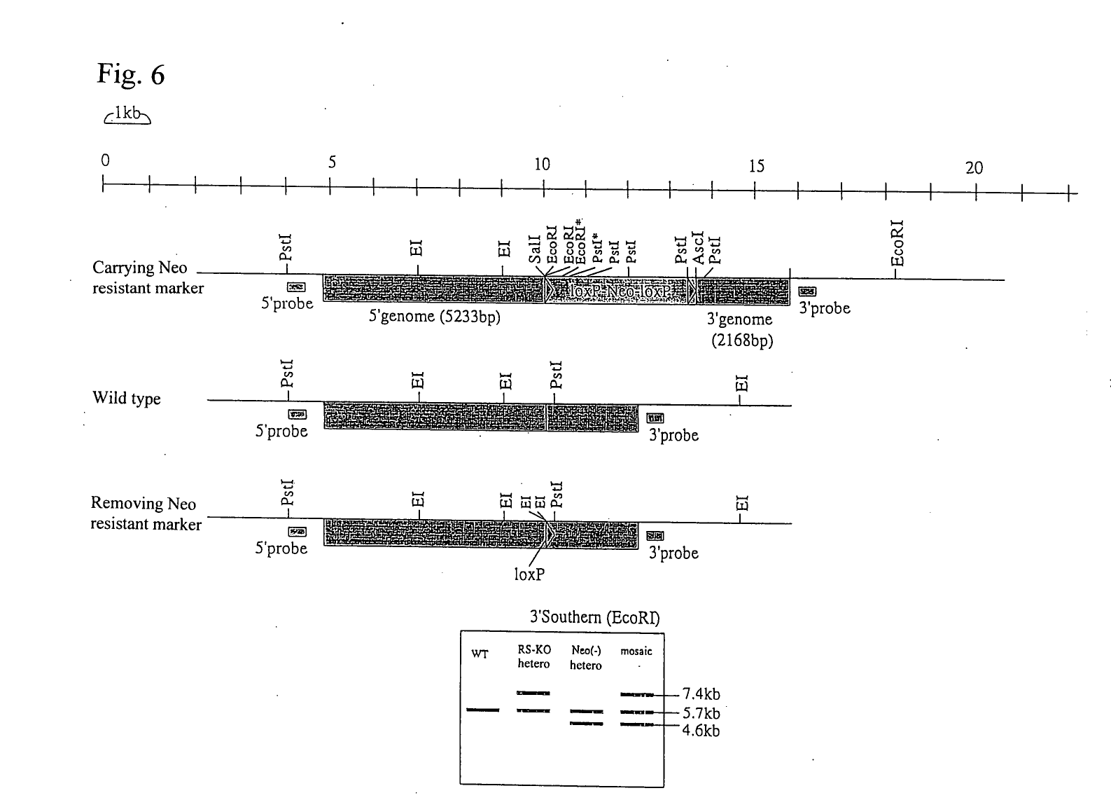

As a result, 5 clones died in the presence of G418. These G418 sensitive cell lines were grown on a 35-mm Petri dish up to confluent state, and 80% of the cells were suspended in 0.5 ml of the stock medium (ES medium+10% DMSO), frozen, and stored at −80° C. The remaining 20% of the cells were seeded on a 12-well plate coated with gelatin and cultured for 2 days. Genomic DNA was prepared from 10⁶ to 10⁷ cells by Puregene DNA isolation kits (Gentra System, USA). Of the G418 sensitive cell lines, the genomic DNAs of RS32#10G- and RS32#15G- cell lines were digested with restriction enzyme EcoRI, separated by agarose gel electrophoresis and subjected to Southern blot. In this manner, the removal of the Neo resistant gene was confirmed by use of the 3′KO-prob as used in Example 4. As a result, 7.4 kb band was observed in the RS32 cell line (RS-KO heterozygote), but not observed in the sensitive cell lines. Instead, 4.6 kb band, which was expected to be observed if the Neo resistant marker was removed, was detected (FIG. 6).

Furthermore, the removal of the Neo resistance marker gene was confirmed by PCR analysis using the following primers. A reaction mixture was prepared in accordance with the instructions of Takara Ex-Taq (Takara Shuzo, Japan), and PCR was performed using the genomic DNA from the G418 sensitive cell line as a template. The reaction conditions of the PCR were: 1 cycle of 94° C. for 3 minutes, 35 cycles of 94° C. for 15 seconds+68° C. for 4 minutes, and 1 cycle of 68° C. for 3 minutes. The reaction mixture was subjected to 0.8% agarose gel electrophoresis to detect an amplified product.

| | |
|---|---|
| Neo(-)loxP FW5: | |
| GGAATTCCGATCATATTCAATAACCCTTAAT | (SEQ ID NO:30) |
| RSwtRV3: | |
| ACTGCCAAGCCCTTAACTTTGTTATCGTAAG | (SEQ ID NO:31) |

When PCR analysis was performed by use of the primers under the same conditions as above, if the Neo resistant marker was present then 4 kb band would be amplified, and if the Neo resistant marker was absent then 430 bp band would be amplified. Since Neo(−)loxP FW5 primer is constituted of the sequence from a plasmid upstream of loxP, wild allele would not be amplified. As a result, the 430 bp band indicating the removal of the Neo resistant marker, was detected in two cell lines, RS32#10G- and RS32#15G-.

From the results mentioned above, it was confirmed that the Neo resistant marker gene has been removed in the obtained G418 sensitive cell line. Then, chimeric mice were produced from RS32#10G- and RS32#15G- cell lines in accordance with the method described in Example 10. As a result, mice which had a chimeric rate of 100% in terms of hair color were obtained. Thus, RS32#10G- and RS32#15G- cell lines were demonstrated to have a high chimera formation potency.

### Example 13

Study on Efficiency of Homologous Recombination in RS Element Targeting Murine ES Cell Lines (RS32#10G- and RS32#15G-) from Which the Neo Resistant Marker Gene (Comprising SV Enhancer) Had Been Removed.

The human EPO targeting vector as prepared in Example 7 was linearized by restriction enzyme NotI (Takara Shuzo, Japan) and introduced into each of the RS element targeting murine ES cell line (RS32#10G- and RS32#15G-) from which the Neo resistant marker gene had been removed, in accordance with the established method (Shinichi Aizawa, ibid). The RS32#10G- and RS32#15G- cell lines were cultured in accordance with the method (Shinichi Aizawa, ibid) using, as a trophocyte, the G418 resistant primary cultured cell (Invitrogen, USA) treated with mitomycin C (Sigma, USA). The amplified RS32#10G- and RS32#15G-cells were independently treated with trypsin and suspended in HBS at 3×10⁷ cells/ml. Then, 0.5 ml of the cell suspension was mixed with 10 μg of vector DNA and placed in a gene pulsar cuvette (distance between electrodes: 0.4 cm; Biorad, USA) and subjected to electroporation (capacity: 960 μF, voltage: 240 V, room temperature). After electroporation, the cells were suspended in 10 ml of the ES medium (Shinichi Aizawa, ibid) and seeded on a 100-mm plastic tissue-culture Petri dish (Falcon; Becton Dickinson, USA) having feeder cells previously seeded therein. After 36 hours, the medium was replaced with fresh ES medium containing 0.8 μg/ml puromycin (Sigma, USA). After 7 days, colonies generated. Of them, 30 colonies were picked up, individually transferred to 24-well plates, and grown up to the confluent state. Two thirds of the grown cells were suspended in 0.2 ml of stock medium (ES medium+10% DMSO, Sigma, USA) and stored at −80° C. The remaining one thirds was seeded on a 12-well gelatin coated plate and cultured for 2 days. From 10⁶-10⁷ cells, genomic DNA was prepared by use of Puregene DNA Isolation Kits (Gentra System, USA). The genomic DNA of the puromycin-resistant cell line was digested with restriction enzyme EcoRI (Takara Shuzo, Japan) and separated by agarose gel electrophoresis. Subsequently, Southern blot was performed by use of, as a probe, a DNA fragment (XhoI to EcoRI, about 1.4 kb, FIG. 5), which was at the 3′ end of the Ig light chain Jκ-Cκ genomic DNA and had been used in the invention described in WO 00/10383 (see Example 48), to detect homologous recombinants. As a result, homologous recombinants were obtained in the rate of 8 of 30 cell lines (26.7%) in the RS32#10G-cell line and in the rate of 2 of 30 cell lines (6.8%) in the RS32#15G- cell line. In the control wild-type TT2F cell, a single band was detected by EcoRI digestion. In the homologous recombinants, a new band was expected to appear below this band (WO 00/10383, see Example 58). Actually, the new band was detected in the puromycin resistant cell line. In short, these clones had human EPO-cDNA inserted downstream of the immunoglobulin κ-chain gene of one allele.

In the RS element targeting murine ES cell lines (RS32#10G- and RS32#15-) from which the Neo resistant marker gene (comprising SV 40 enhancer) was previously removed, the rate of homologous recombinants was 10 of 60 cell lines (16.7%) in sum of the results of two clones when the human EPO targeting vector (FIG. 4) was used. On the other hand, the rate was 15 of 89 cell lines (16.9%, Example 8) in the wild type TT2F cell line, and 15 of 24 cell lines (62.5%, Example 9) in the RS32 cell line having the Neo resistant marker gene. This means that high efficiency of homologous recombination of said cell line (RS32) having the Neo resistant marker gene in the Cκ region was not achieved by removal of the Neo resistant marker gene. This suggests that particularly the presence of SV 40 enhancer of the Neo resistant marker gene inserted in the RS element region improves the homologous recombination efficiency in the Cκ region located at 25 Kb upstream thereof. On the other hand, it was also suggested that deletion of the RS element itself did not affect the efficiency of homologous recombination. These results demonstrate that the efficiency of homologous recombination could be improved by modifying the genomic region, which was not contained in the targeting vector but was present in the vicinity of the target region.

### Example 14

Preparation of pRS-KOSV40PE Vector for Targeting Murine RS Element

The murine RS element targeting vector pRS-KOSV40PE (FIG. 7) was constructed by inserting an SV40 enhancer/promoter sequence (SV40PE) into the AscI site (located outside the loxP-Neo-loxP sequence) of the murine RS element targeting vector, (pBlueRS-LoxP-Neo-DT-A-3′KO-5′KO) prepared in Example 1. The SV40 enhancer/promoter sequence (SV40PE; Yamaizumi, protein/nucleic acid/enzyme, Vol. 28, No. 14, p. 1599-, 1983, published by Kyoritsu Shuppan, Japan), which comprises an enhancer sequence consisting of tandem repeats of a 72-bp unit, a replication origin, and the early in RNA promoter, is about 0.35 kb region contained in the Neo resistant marker gene unit of the vector pBlueRS-LoxP-Neo-DT-A-3′KO-5′KO (FIG. 8). The SV40 PE fragment to be inserted into the aforementioned AscI site may be prepared by amplifying a fragment by PCR using primers designed such that both ends of the SV40PE fragment have the AscI site and digesting the amplified fragment with Asc I. The RS element targeting vector (pRS-KOSV40PE) thus constructed was then introduced into murine ES cells by the method described in Example 4, and the G418 resistant cell line obtained was analyzed by the method described in Example 4. As a result, it was found that the ES cell line contains no chromosomal region having the murine RS element, and instead, contains the DNA fragment having the SV40PE sequence and the Neo resistant marker gene (having the LoxP sequence at both ends) mutually connected (FIG. 9: recombinant). The karyotype of the ES cell line thus obtained was analyzed in the same manner as in Example 4. As a result, it was confirmed that no abnormal karyotype was detected in the obtained ES cell line.

(1) Preparation of Full Length SV40PE Fragment

The following primers were synthesized in order to amplify the region consisting of early promoter/enhancer/replication origin derived from SV40 viral genome by PCR based on the pBlueRS-LoxP-Neo-DT-A-3′KO-5′KO vector.

| | |
|---|---|
| SV40PE-F: | |
| GGCGCGCCGCTGTGGAATGTGTGTCAGT | (SEQ ID NO:32) |
| SV40PE-R: | |
| GGCGCGCCAAGCTTTTGCAAAAGCCTAG | (SEQ ID NO:33) |

A reaction solution was prepared using KOD-plus- (TOYOBO, Japan) in accordance with the instructions attached thereto. To the reaction solution (50 μl), the two types of primers as mentioned above (10 pmol each) and the pBlueRS-LoxP-Neo-DT-A-3′KO-5′KO vector serving as a template were added. After the reaction mixture was maintained at 94° C. for 2 minutes, a PCR cycle consisting of 94° C. for 20 seconds, 60° C. for 20 seconds and 68° C. for 30 seconds was repeated 30 times. Amplified fragments of 361 bp were digested by restriction enzyme AscI and separated by 2% gel electrophoresis. From the recovered gel, SV40PE/AscI fragment wa recovered by QIAquick Gel Extraction Kit (Qiagen, Germany) in accordance with the instructions attached thereto.

(2) Construction of pRS-KOSV40PE

The pBlueRS-LoxP-Neo-DT-A-3′KO-5′KO vector was digested with restriction enzyme AscI and separated by 0.8% agarose gel electrophoresis. About 15 kb of an enzyme-treated fragment was recovered from the gel by QIAquick Gel Extraction Kit (Qiagen) in accordance with the instructions. The ends of the AscI fragment of pBlueRS-LoxP-Neo-DT-A-3′KO-5′KO vector thus obtained were dephosphorylated with alkaline phosphatase derived from the fetal bovine intestine. Into the resultant vector fragment, the DNA fragment prepared in (1) above were inserted, and then the vector was introduced into Escherichia coli XL10-Gold Ultracompetent Cells (STRATAGENE, USA). DNA was prepared from the obtained transformant and the nucleotide sequence of the ligated portion was confirmed. In this manner, the pRS-KOSV40PE vector was obtained (FIG. 7).

### Example 15

Preparation of pRS-KOSV40PE/NotI Vector for Electroporation

60 μg of the pRS-KOSV40PE vector was digested with NotI at 37° C. for 5 hours in a buffer (H buffer for restriction enzyme; Roche Diagnostics, Germany) supplemented with spermidine (1 mM pH7.0; Sigma, USA). After extraction with phenol/chloroform, 2.5 volumes of 100% ethanol and 0.1 volumes of 3M sodium acetate were added to the resultant mixture and stored at −20° C. for 16 hours. The vector that was single-standed with NotI was centrifugally collected and sterilized by adding 70% ethanol. Then, the 70% ethanol was removed in a clean ventilator, and the residue was air-dried for one hour. To this matter was added an HBS solution to prepare a 0.5 μg/μl DNA solution, which was stored at room temperature for one hour. In this way, pRS-KOSV40PE/NotI vector for electroporation was prepared.

### Example 16

Obtaining Murine ES Cell Targeted by pRS-KOSV40PE

The pRS-KOSV40PE/NotI vector prepared in Example 15 was introduced into murine ES cell TT2F (Yagi et al., Analytical Biochemistry, 214:70, 1993) in accordance with the established method (Shinichi Aizawa, ibid). The TT2F cells were cultured in accordance with the method (Shinichi Aizawa, as above) using, as a trophocyte, the G418 resistant primary culture cell (Invitrogen, USA) which had been treated with mitomycin C (Sigma, USA). The TT2F cells grown were treated with trypsin and suspended in HBS at 3×10⁷ cells/ml. Thereafter, 0.5 ml of the cell suspension was mixed with 10 μg of vector DNA, loaded in a gene pulsar cuvette (distance between electrodes: 0.4 cm; Biorad, USA), and subjected to electroporation (capacity: 960 μF, voltage: 240 V, room temperature). After electroporation, the cells were suspended in 10 ml of ES medium and seeded on a 100 mm plastic tissue-culture Petri dish (Falcon; Becton Dickinson, USA) having feeder cells previously seeded. After 24 hours, the medium was replaced with a fresh ES medium containing 200 μg/ml G418 (Sigma, USA). After 7 days, colonies generated were picked up, individually transferred to a 24-well plate, and grown up to the confluent state. Two thirds of the grown cells were suspended in 0.2 ml of a stock medium (ES medium+10% DMSO; Sigma, USA) and stored at −80° C. The remaining one thirds was seeded on a 12-well gelatin coated plate and cultured for 2 days. From 10⁶-10⁷ cells, genomic DNA was prepared by use of Puregene DNA Isolation Kits (Gentra System, USA). The genomic DNA from the neomycin (G418)-resistant TT2F cells was digested with restriction enzyme EcoRI (Takara Shuzo, Japan) and resolved by agarose gel electrophoresis. Subsequently, Southern blot was performed by use of, as a probe, a DNA fragment (3′KO-prob, see Example 3, FIG. 2), which was located downstream of the 3′ homologous region of the targeting vector, thereby detecting homologous recombinants. In the wild-type TT2F cell, a single band (about 5.7 Kb) was detected by EcoRI digestion. In the homologous recombinant, the detection of two bands (about 5.7 Kb and about 7.8 Kb) was expected. However, indeed, a new band of about 7.8 Kb (about 7.4 kb+about 0.35 kb SV40PE fragment) was detected in part of the G418 resistant cell line. Furthermore, the genomic DNA of the clones in which homologous recombination was confirmed by Southern analysis using 3′KO-prob was digested with restriction enzyme PstI (Takara Shuzo., Japan) and resolved by 0.8% agarose gel electrophoresis. Subsequently, Southern blot was performed by use of, as a probe, the DNA fragment (5′KO-prob, see Example 3, FIG. 2) located upstream of the 5′ homologous region of the targeting vector, thereby detecting homologous recombinants. In the wild-type TT2F cell, a single band (about 6.1 Kb) was detected by PstI digestion. In the homologous recombinant, the detection of two bands (about 6.7 Kb and about 6.1 Kb) was expected. However, indeed, a new band of about 6.7 Kb was detected in the G418 resistant cell line. These clones were devoid of the chromosomal region containing the murine RS element, and instead, the Neo-resistant marker and the SV40PE fragment were inserted therein. As a result of Southern analysis using 3′KO-prob and 5′KO-prob, it was found that 10 cell lines (25%) out of 40 cell lines were homologous recombinants when pRS-KOSV40PE was linearized by restriction enzyme NotI. The karyotype of murine ES cells targeted by pRS-KOSV40PE was analyzed in accordance with the method described in Bio-manual series 8, gene targeting (Shinichi Aizawa, as above). As a result, it was confirmed that no abnormal karyotype was detected in the ES cells targeted.

### Example 17

Removal of Neomycin-Resistant Marker Gene from Murine ES Cell Line Targeted by pRS-KOSV40PE

The Neo resistant marker gene was removed from the murine ES cell lines (G418:Neo-resistant cell lines), namely RSSV40PE#8 and RSSV40PE#18, targeted by pRS-KOSV40PE (obtained in Example 16) confirmed to have a normal karyotype, in accordance with the following procedure. Use was made of expression vector pBS185 which contains the Cre recombinase gene, responsible for causing a site-directed recombination between loxP sequences inserted into both sides of the Neo resistance marker gene. The expression vector pBS185 was introduced into each of the RSSV40PE#8 and RSSV40PE#18 cell lines in accordance with the method described by Shinichi Aizawa (as above). The cells were treated with trypsin and suspended in HBS at 2.5×10⁷ cells/ml. To the suspension, 30 μg of pBS185 DNA was added and subjected to electroporation using gene pulsar (Biorad). More specifically, a voltage of 250V (960 μF in capacity) was applied to a 4 mm-long electroporation cell (165-2088; Biorad) containing said suspension. The electroporated cells were suspended in 5 ml of ES medium and seeded on a 100 mm tissue-culture plastic Petri dish having feeder cells previously seeded. After 2 days, the cells were treated with trypsin and seeded again on three 100 mm Petri dishes having feeder cells seeded in a rate of 100, 300 or 800 cells per dish, respectively. After 7 days, colonies generated were picked up, treated with trypsin, and divided into two portions. One of them was seeded on a 48-well plate having feeder cells seeded, whereas the other was seeded on a 48-well plate coated only with gelatin. The latter one was cultured in a medium containing 200 μg/ml of G418 for 3 days. G418 resistance was determined based on the survival of cells. The resultant G418 sensitive cells were grown on a 35-mm Petri dish up to confluent state and 80% of the cells were suspended in 0.5 ml of stock medium (ES medium+10% DMSO) and stored at −80° C. in a freezer. The remaining 20% of the cells were seeded on a 12-well plate coated with gelatin and cultured for 2 days. Genomic DNA was prepared from 10⁶-10⁷ cells by Puregene DNA isolation kit (Gentra System). Of the G418 sensitive cell lines derived from RSSV40PE#8, two cell lines of RSSV40PE8G-#32 and RSSV40PE8G-#36 were chosen. Of the G418 sensitive cell lines derived from RSSV40PE#18, two cell lines of RSSV40PE18G-#37 and RSSV40PE18G-#39 were chosen. The genomic DNAs of these 4 cell lines were digested with EcoRI, resolved by agarose gel electrophoresis, and analyzed by the Southern blot with 3′KO-prob as used in Example 4. In this manner, removal of the Neo resistant gene was confirmed. As a result, although the band of about 7.8 kb (about 7.4 kb+about 0.35 kb SVPE fragment) was observed in both of RSSV40PE#8 and RSSV40PE#18 cell lines, such a band was not observed in the 4 types of sensitive cell lines. Instead, a band of about 4.9 kb (about 4.6 kb+about 0.35 kb SVPE fragment), which was expected to be observed when the Neo resistant marker was removed, was detected (FIG. 6).

From the results above, it was confirmed that the Neo resistant marker gene was removed from the 4 types of G418 sensitive cell lines without fail (FIG. 9, Neo(−)).

### Example 18

Preparation of Murine RS Element Targeting Vector pRS-KOSV4072bp

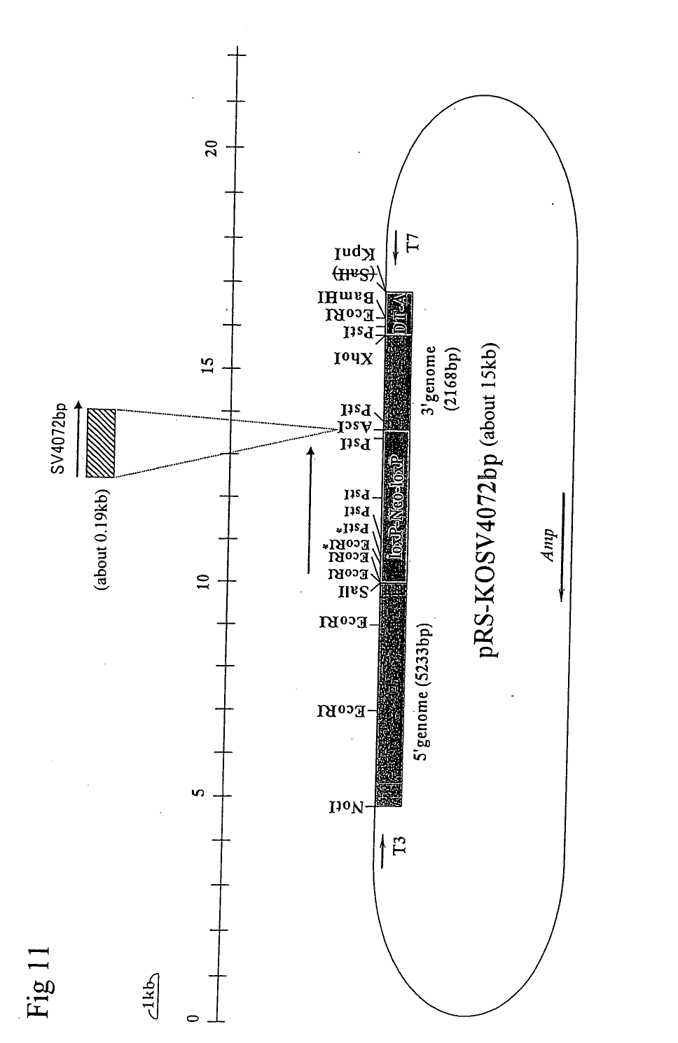

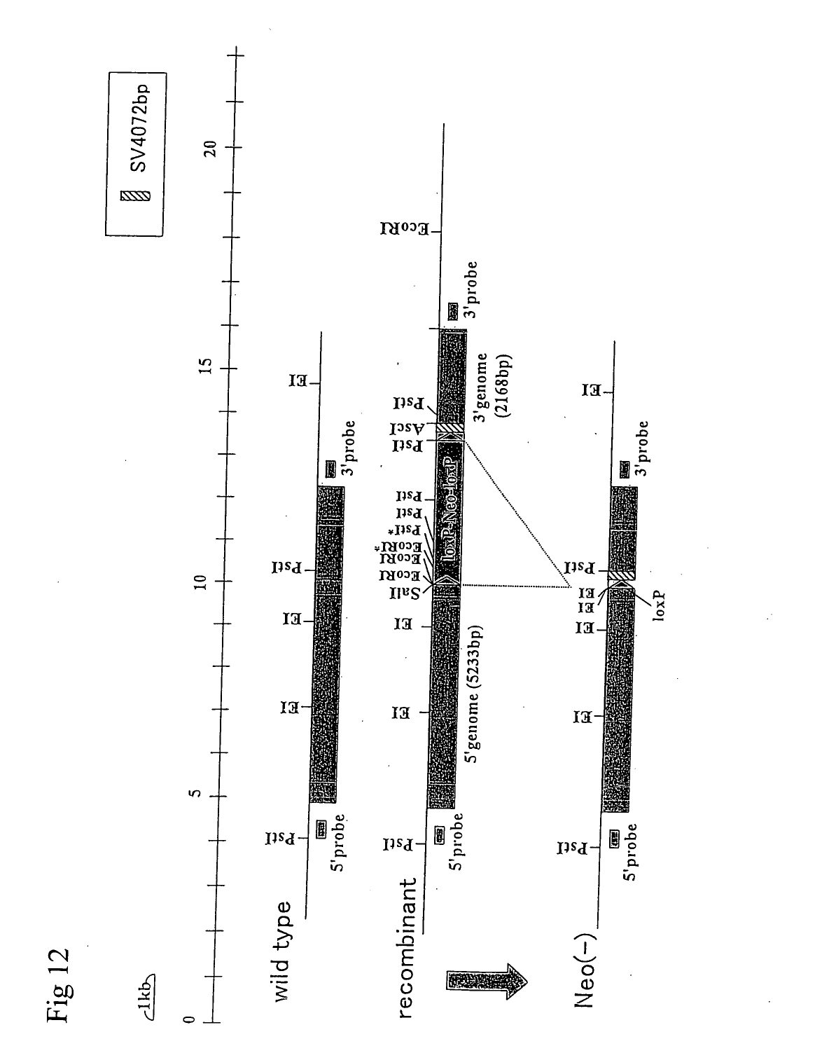

The SV40 enhancer/promoter sequence (SV40PE; Yamaizumi, protein/nucleic acid/enzyme, Vol. 28, No. 14, p. 1599-, 1983, published by Kyoritsu Shuppan, Japan) containing an enhancer sequence consisting of tandem repeats of a 72-bp unit, a replication origin, and the early mRNA promoter, is about 0.35 kb region contained in the Neo resistant marker gene unit of pBlueRS-LoxP-Neo-DT-A-3′KO-5′KO (FIG. 8). A murine RS element targeting vector (pRS-KOSV4072bp) (FIG. 11) was constructed by inserting SV40 enhancer sequence (SV4072bp) alone into the AscI site of the murine RS element targeting vector, pBlueRS-LoxP-Neo-DT-A-3′KO-5′KO constructed in Example 1. The SV 4072 bp fragment inserted into the AscI site may be prepared by amplifying a fragment by PCR using primers designed such that both ends of the SV4072bp fragment have the AscI sites and digesting the amplified fragment with AscI. The RS element targeting vector (pRS-KOSV4072bp) thus constructed was introduced into murine ES cells by the method as described in Example 4 and the G418 resistant cell line obtained was analyzed by the method as described in Example 4. As a result, it was found that ES cell line contains no chromosomal region containing the murine RS element, and instead, contains the DNA fragment having the SV4072bp sequence and the Neo resistant marker gene (having the LoxP sequence at both ends) mutually connected (FIG. 12: recombinant). The karyotype of the ES cell line thus obtained was analyzed as in Example 4. As a result, it was confirmed that no abnormal karyotype was detected in the obtained ES cell line.

(1) Preparation of SV40 Enhancer (Tandem Repeats of 72-bp Unit×2) Fragment

The following primers were designed to amplify the enhancer region (having tandem repeats of 72-bp unit×2) derived from SV40 viral genome by PCR based on the pBlueRS-LoxP-Neo-DT-A-3′KO-5′KO vector.

| | |
|---|---|
| SV4072bp-F: | |
| GGCGCGCC GTG TGT CAG TTA GGG TGT GG | (SEQ ID NO:34) |
| SV4072bp-R: | |
| GGCGCGCC AGG GGC GGG ACT ATG GTT GC | (SEQ ID NO:35) |

A reaction mixture was prepared using KOD-plus- (TOYOBO, Japan) in accordance with the instructions attached thereto. To the reaction mixture (50 μl), the two types of primers as mentioned above (10 pmol each) and the pBlueRS-LoxP-Neo-DT-A-3′KO-5′KO vector as a template were added. After the reaction mixture was maintained at 94° C. for 2 minutes, a PCR cycle consisting of 94° C. for 20 seconds, 62° C. for 20 seconds and 68° C. for 20 seconds was repeated 30 times. Amplified fragment of 186 bp was digested by restriction enzyme AscI and separated by 2% gel electrophoresis. From the recovered gel, SV40 enhancer (AscI) fragment was recovered by QIAquick Gel Extraction Kit (Qiagen) in accordance with the instructions.

(2) Construction of pRS-KOSV4072bp Vector

The ends of the AscI fragment of the pBlueRS-LoxP-Neo-DT-A-3′KO-5′KO vector obtained in Example 14-(2) were dephosphorylated. The SV40 enhancer AscI fragment prepared in the step (1) was inserted into the resultant vector fragment, and then the vector was introduced into Escherichia coli XL10-Gold Ultracompetent Cells. DNA was prepared from the obtained transformants and the nucleotide sequence of the ligated portion was confirmed. In this manner, the pRS-KOSV4072bp vector was obtained (FIG. 11).

### Example 19

Preparation of pRS-KOSV4072bp Vector for Electroporation

60 μg of pRS-KOSV4072bp vectors was digested with NotI at 37° C. for 5 hours in a buffer (H buffer for restriction enzyme, Roche Diagnostics) supplemented with spermidine (1 mM pH7.0; Sigma, USA). After extraction with phenol/chloroform, 2.5 volumes of 100% ethanol and 0.1 volumes of 3M sodium acetate were added to the resultant mixture and stored at −20° C. for 16 hours. The vector single-standed with NotI was centrifugally collected and sterilized by adding 70% ethanol. Then, 70% ethanol was removed in a clean ventilator and the residue was air-dried for one hour. To the dried matter was added an HBS solution to prepare a 0.5 μg/μl DNA solution and stored at room temperature for one hour. In this way, the pRS-KOSV4072bp/NotI vectors for electroporation were prepared.

### Example 20

Obtaining Murine ES Cell Targeted by pRS-KOSV4072bp

The pRS-KOSV4072bp/NotI vector as prepared in Example 19 was introduced into murine ES cells TT2F (Yagi et al., Analytical Biochemistry, 214:70, 1993) in accordance with the established method (Shinichi Aizawa, ibid). The TT2F cell was cultured in accordance with the method (Shinichi Aizawa, as above) using, as a trophocyte, the G418 resistant primary culture cell (Invitrogen, USA) treated with mitomycin C (Sigma, USA). The TT2F cells grown were treated with trypsin and suspended in HBS at 3×10⁷ cells/ml. Thereafter, 0.5 ml of the cell suspension was mixed with 10 μg of the vector DNA, loaded in a gene pulsar cuvette (distance between electrodes: 0.4 cm; Biorad, USA) and subjected to electroporation (capacity: 960 μF, voltage: 240 V, room temperature). After electroporation, the cells were suspended in 10 ml of ES medium and seeded on a 100 mm plastic tissue-culture Petri dish (Falcon, Becton Dickinson, USA) having feeder cells seeded. After 24 hours, the medium was replaced with a fresh ES medium containing 200 μg/ml G418 (Sigma, USA). The colonies generated after 7 days were picked up, individually transferred to a 24-well plate, and grown up to the confluent state. Two thirds of the grown cells were suspended in 0.2 ml of stock medium (ES medium+10% DMSO; Sigma, USA) and stored at −80° C. The remaining one thirds was seeded on a 12-well gelatin coated plate and cultured for 2 days. From 10⁶-10⁷ cells, genomic DNA was prepared by use of Puregene DNA Isolation Kits (Gentra System, USA). The genomic DNA of the G418-resistant TT2F cells was digested with restriction enzyme EcoRI (Takara Shuzo, Japan) and resolved by 0.8% agarose gel electrophoresis. Subsequently, Southern blot was performed by use of, as a probe, a DNA fragment (3′KO-prob, Example 3, FIG. 2), which was located downstream of the 3′ homologous region of the targeting vector to detect homologous recombinants. In the wild-type TT2F cells, a single band (about 5.7 Kb) was detected by EcoRI digestion. In the homologous recombinants, the detection of two bands (about 5.7 Kb and about 7.6 Kb) was expected. However, actually a new band of about 7.6 Kb (about 7.4 kb+about 0.19 kb SV4072bp fragment) was detected in part of the G418 resistant cell line. Furthermore, the genomic DNA of the clones which were confirmed as homologous recombinants by Southern analysis using 3′KO-prob, was digested with restriction enzyme PstI (Takara Shuzo, Japan) and separated by 0.8% agarose gel electrophoresis. Subsequently, Southern blot was performed by use of, as a probe, the DNA fragment (5′KO-prob, see Example 3, FIG. 2) located upstream of the 5′ homologous region of the targeting vector to detect homologous recombinants. In the wild-type TT2F cell, a single band (about 6.1 Kb) was detected by PstI digestion. In the homologous recombinants, the detection of two bands (about 6.7 Kb and about 6.1 Kb) was expected. However, indeed, a new band of about 6.7 Kb was detected in the G418 resistant cell line. These clones are devoid of the chromosomal region containing the murine RS element, and instead, the Neo-resistant marker gene and SV40 enhancer (containing tandem repeats of 72-bp unit×2) were inserted therein. As a result of Southern analysis using 3′KO-prob and 5′KO-prob, it was found that 4 cell lines (10%) out of 40 cell lines were homologous recombinants when pRS-KOSV4072bp was linearized by restriction enzyme NotI. The karyotype of the murine ES cell targeted by pRS-KOSV4072bp was analyzed by the method described by Shinichi Aizawa (as above). As a result, it was confirmed that no abnormal karyotype was detected in the ES cell targeted.

### Example 21

Removal of Neomycin-Resistant Marker Gene from Murine ES Cell Line Targeted by pRS-KOSV4072bp

The Neo resistant marker gene was removed from the murine ES cell lines (G418: Neo-resistant cell lines), namely RSSV4072bp#37 and RSSV4072bp#38 (obtained in Example 20), confirmed to have a normal karyotype and targeted by pRS-KOSV4072bp, in accordance with the following procedure (FIG. 12). Use was made of expression vector pBS185, which contains the Cre recombinase gene, responsible for causing site-directed recombination between loxP sequences inserted onto both sides of the Neo resistance marker gene. The expression vector pBS185 was introduced into each of the RSSV4072bp#37 and RSSV4072bp#38 cell lines in accordance with the method described by Shinichi Aizawa (as above). The cells were treated with trypsin and suspended in HBS at 2.5×10⁷ cells/ml. To the suspension, 30 μg of pBS185 DNA was added and subjected to electroporation using gene pulsar (Biorad). More specifically, a voltage of 250V (960 μF in capacity) was applied to a 4 mm-long electroporation cell (165-2088; Biorad) containing the suspension. Then, the cells treated by electroporation were suspended in 5 ml of ES medium and seeded on a 100 mm tissue-culture plastic Petri dish having feeder cells seeded. After 2 days, the cells were treated with trypsin and seeded again in three 100 mm Petri dishes having feeder cells seeded in a rate of 100, 300 or 800 cells per dish, respectively. After 7 days, the colonies generated were picked up, treated with trypsin, and divided into two portions. One of them was seeded on a 48-well plate having feeder cells seeded while the other was seeded on a 48-well plate coated only with gelatin. The latter one was cultured in a medium containing 200 μg/ml of G418 for 3 days. G418 resistance was determined based on the survival of cells.

The resultant G418 sensitive cells were grown on a 35-mm Petri dish up to confluent state and 80% of the cells were suspended in 0.5 ml of stock medium (ES medium+10% DMSO) and stored at −80° C. in a freezer. The remaining 20% of the cells were seeded on a 12-well plate coated with gelatin and cultured for 2 days. Genomic DNA was prepared from 10⁶-10⁷ cells by Puregene DNA isolation kit (Gentra System). Of the G418 sensitive cell lines derived from RSSV4072bp#37, two cell lines of RSSV4072bp37G-#4 and RSSV4072bp37G-#4 were chosen. Of the G418 sensitive cell lines derived from RSSV4072bp#38, two cell lines, RSSV4072bp38G-#26 and RSSV4072bp37G-#28 were chosen. The genomic DNA of the 4 cell lines were digested with restriction enzyme EcoRI, separated by agarose gel electrophoresis, and analyzed by the Southern blot with 3′KO-prob as used in Example 4. In this manner, removal of the Neo resistant gene was confirmed. As a result, although the band of about 7.6 kb (about 7.4 kb+about 0.19 kb SV4072bp fragment) was observed in RSSV4072bp#37 and RSSV4072bp#38; it was not observed in these 4 types of sensitive cell lines. Instead, a band of about 4.8 kb (about 4.6 kb+about 0.19 kb SV4072bp fragment), which was expected to be observed when the Neo resistant marker was removed, was detected (FIG. 6).

From the results above, it was confirmed that the Neo resistant marker was removed from the 4 types of G418 sensitive cell lines.

### Example 22

Construction of CκP2TPO(DT−) Vector

pCκP2TPOKI vector (International Publication WO 2003/041495) was digested with restriction enzymes KpnI and XhoI, and about 20 kb fragment was separated by 0.8% agarose gel electrophoresis. From the recovered gel, Cκ TPO DT− fragments were recovered by QIAquick Gel Extraction Kit (QIAGEN) in accordance with the instructions, blunt ended with Blunting high (TOYOBO, Japan), self-circularized, and introduced into Escherichia coli XL10-Gold Ultracompetent Cells (STRATAGENE, USA). DNA was prepared from the obtained transformant and the nucleotide sequence of the ligated portion was confirmed. In this manner, the pCκP2TPO(DT−) vector was obtained.

### Example 23

Insertion of Human FGF7 Gene into CκP2 Targeting Vector

(1) Preparation of Human FGF7-DNA Fragment

| | | |
|---|---|---|
| FGF7SalIFW: | ACGCGTCGACCACCATGCACAAATGGATACTGACATGGA | (SEQ ID NO:36) |
| FGF7NheIRV: | CTAGCTAGCTTAAGTTATTGCCATAGGAAGAAAG | (SEQ ID NO:37) |

A reaction mixture was prepared using KOD-plus- (TOYOBO, Japan) in accordance with the instructions attached thereto. To the reaction mixture (50 μl), the two types of primers as mentioned above (10 pmol each) and the human FGF7 cDNA as a template were added. After the reaction mixture was maintained at 94° C. for 2 minutes, a PCR cycle consisting of 94° C. for 15 seconds and 68° C. for 1 minute was repeated 30 times. Amplified fragment of 603 bp was separated by 0.8% gel electrophoresis. From the recovered gel, the amplified fragment was recovered by QIAquick Gel Extraction Kit (Qiagen) in accordance with the instructions attached thereto. The amplified fragment recovered was digested with SalI and NheI and separated by 0.8% agarose gel electrophoresis. From the recovered gel, fragments digested with enzymes were recovered by QIAquick Gel Extraction Kit (Qiagen) in accordance with the instructions. After pBluescriptIISK(−) (STRATAGENE, USA) was digested with SalI and NheI and separated and purified by 0.8% agarose gel electrophoresis, the ends of pBluescriptIISK(−) were dephosphorylated with alkaline phosphatase derived from the fetal bovine intestine. The DNA fragments recovered above were inserted into the obtained pBluescriptIISK(−), which was then introduced into Escherichia coli DH5α. DNA was prepared from the obtained transformants and the inserted fragment was sequenced. Clones having no mutation due to PCR were selected, digested with XhoI, and separated by 0.8% agarose gel electrophoresis. From the agarose gel thus recovered, the human FGF7-DNA fragment was recovered by QIAquick Gel Extraction Kit (Qiagen) in accordance with the instructions.

(2) Construction of pCκP2FGF Vector

The CκP2 targeting vector (FIG. 3) was digested with SalI and NheI and the ends of the vector were dephosphorylated with alkaline phosphatase derived from the fetal bovine intestine. Into the vector was introduced the human FGF7-cDNA fragment prepared in (1) above. The obtained vector was introduced into Escherichia coli XL10-Gold Ultracompetent Cells (STRATAGENE, USA). DNA was prepared from transformants and the nucleotide sequence of the ligated portion was confirmed. In this manner, the CκP2 human FGF7 target vector (pCκP2FGF7) was obtained.

### Example 24

Preparation of pCκP2TPO Vector for Electroporation

60 μg of pCκP2TPO vector (International Publication WO 2003/041495) was digested with NotI at 37° C. for 5 hours in a buffer (H buffer for restriction enzyme; Roche Diagnostics) supplemented with spermidine (1 mM pH7.0; Sigma, USA). After extraction with phenol/chloroform, 2.5 volumes of 100% ethanol and 0.1 volumes of 3M sodium acetate were added to the resultant mixture and stored at −20° C. for 16 hours. The vector single-standed with NotI was centrifugally collected and sterilized by adding 70% ethanol. Then, 70% ethanol was removed in a clean ventilator and the residue was air-dried for one hour. To the obtained matter, an HBS solution was added to prepare a 0.5 μg/μl DNA solution and stored at room temperature for one hour. In this way, the pCκP2TPO vector for electroporation was prepared.

### Example 25

Preparation of pCκP2TPO(DT−) Vector for Electroporation

60 μg of pCκP2TPO(DT−) vector as constructed in Example 22 was digested with NotI at 37° C. for 5 hours in a buffer (H buffer for restriction enzyme; Roche Diagnostics) supplemented with spermidine (1 mM pH7.0; Sigma, USA). After extraction with phenol/chloroform, 2.5 volumes of 100% ethanol and 0.1 volumes of 3M sodium acetate were added to the resultant mixture and stored at −20° C. for 16 hours. The vector single-standsed with NotI was centrifugally collected and sterilized by adding 70% ethanol. Then, 70% ethanol was removed in a clean ventilator and the residue was air-dried for one hour. To the obtained matter, an HBS solution was added to prepare a 0.5 μg/μl DNA solution and stored at room temperature for one hour. In this way, the pCκP2TPO(DT−) vector for electroporation was prepared.

### Example 26

Preparation of pCκP2FGF7 Vector for Electroporation

60 μg of pCκP2FGF7 vector as constructed in Example 23 was digested with NotI at 37° C. for 5 hours in a buffer (H buffer for restriction enzyme; Roche Diagnostics) supplemented with spermidine (1 mM pH7.0; Sigma, USA). After extraction with phenol/chloroform, 2.5 volumes of 100% ethanol and 0.1 volumes of 3M sodium acetate were added to the resultant mixture and stored at −20° C. for 16 hours. The vector single-standed with NotI was centrifugally collected and sterilized by adding 70% ethanol. Then, 70% ethanol was removed in a clean ventilator and the residue was air-dried for one hour. To the obtained matter, an HBS solution was added to prepare a 0.5 μg/μl DNA solution and stored at room temperature for one hour. In this way, the pCκP2FGF7 vector for electroporation was prepared.

### Example 27

Obtaining ES Cell Having Human TPO Gene Introduced by pCκP2TPO Vector

To obtain a murine ES cell line having the human TPO-cDNA which was introduced by homologous recombination downstream of the immunoglobulin κ light-chain gene, the pCκP2TPO vector as prepared in Example 24 was introduced into each of the wild-type murine ES cells, namely TT2F-F8 (Yagi et al., Analytical Biochemistry, 214:70, 1993), RS32 cell line (Example 4), RS32#15G(−) cell line (Example 12) and RSSV40PE8G(−)#36 cell line (Example 17) in accordance with the established method (Shinichi Aizawa, ibid). Culturing murine ES cells was performed in accordance with the method (Shinichi Aizawa, as above) using, as a trophocyte, the G418 resistant primary culture cell (Invitrogen, USA) treated with mitomycin C (Sigma, USA). First, the TT2F cell grown was treated with trypsin and suspended in HBS at 3×10⁷ cells/ml. Thereafter, 0.5 ml of the cell suspension was mixed with 10 μg of vector DNA, loaded in a gene pulsar cuvette (distance between electrodes: 0.4 cm; Biorad, USA), and subjected to electroporation (capacity: 960 μF, voltage: 240 V, room temperature). After electroporation, the cells were suspended in 10 ml of ES medium (Shinichi Aizawa, ibid) and seeded on a 100 mm plastic tissue-culture Petri dish (Falcon; Becton Dickinson, USA) having feeder cells seeded. After 36 hours, the medium was replaced with a fresh ES medium containing 0.8 μg/ml puromycin Sigma, USA). After 7 days, colonies generated. Of them, 40 (TT2F-F8), 12 (RS32), 72 (RS32#15G-) and 72 (RSSV40PE8G-#36) colonies were picked up, individually transferred to 24-well plates, and grown up to confluent state. Two thirds of the grown cells were suspended in 0.2 ml of stock medium (ES medium+10% DMSO; Sigma, USA) and stored at −80° C. The remaining one thirds was seeded on a 12-well gelatin coated plate and cultured for 2 days. From 10⁶-10⁷ cells, genomic DNA was prepared by use of Puregene DNA Isolation Kits (Gentra System, USA). The genomic DNA of each puromycin-resistant ES cell was digested with restriction enzyme EcoRI (Takara Shuzo Co., Ltd., Japan) and separated by 0.8% agarose gel electrophoresis. Subsequently, Southern blot was performed by use of, as a probe, the DNA fragment (XhoI-EcoRI, about 1.4 kb, FIG. 5) which was at the 3′ end of the Ig light chain Jκ-Cκ genomic DNA and which was used in the invention described in WO 00/10383 (see Example 48), thereby detecting homologous recombinants (HRs). The results are shown in Table 1.

TABLE 1

| Sequence present in RS region | | | | | | | |
|---|---|---|---|---|---|---|---|
| RS | Neo resistant | | | | Number of | Number of | |
| sequence | marker | SV40PE | SVE | Cell line | cells analyzed | HRs | % HR |
| ∘ | x | x | x | TT2-F8 | 40 | 3 | 8% |
| x | ∘ | x | x | RS32 | 12 | 4 | 33% |
| x | x | x | x | RS32#15G- | 72 | 6 | 8% |
| x | x | ∘ | x | RSSV40PE8G-#36 | 72 | 40 | 56% |

The percentage of homologous recombinants was 8%, when pCκP2TPO vector was used in the RS element targeting murine ES cell line (RS32#15G-) form which the Neo resistant marker gene (containing SV enhancer) was removed. The percentage of homologous recombinants was 8% in the wild type TT2F-F8 cell line; while 33% in the RS32 cell line carrying the Neo resistant marker gene. Thus, it was found that the homologous recombination efficiency of the cell line (RS32) having the neo resistant marker gene in the Cκ region was higher than that of the wild type ES cell line (TT2-F8), as in the case of human EPO targeting vector (Example 13). It was further shown that the high homologous recombination efficiency disapperaed after removal of the Neo resistant marker gene (RS32#15G-). More importantly, high homologous recombination efficiency (56%) was observed in the cell line (RSSV40PE8G-#36) from which the Neo resistant marker gene in the RS region was removed and in which SV40PE sequence was remained. This demonstrates that the presence of the SV40 enhancer/promoter (SV40PE) sequence inserted into the RS element region improved a homologous recombination efficiency in the Cκ region that was at 25-kb upstream therefrom. These results suggest that the efficiency of homologous recombination in a target region can be enhanced by inserting the SV40 enhancer/promoter (SV40PE) sequence into a genomic region in the vicinity of the target region, even though SV40PE is not contained in the targeting vector.

### Example 28

Obtaining ES Cell Having the Human TPO Gene Introduced by pCκP2TPO(DT−) Vector

To obtain a murine ES cell line having the human thrombopoietin (TPO)-cDNA which was introduced downstream of the immunoglobulin κ light-chain gene by homologous recombination, the pCκP2TPO(DT−) vector as prepared in Example 25 was introduced into each of the RS32 cell line (Example 4), RS32#15G(−) cell line (Example 12) and RSSV40PE8G(−)#36 cell line (Example 17) in accordance with the established method (Shinichi Aizawa, ibid). Culturing murine ES cells was performed in accordance with the method (Shinichi Aizawa, as above) using, as a trophocyte, the G418 resistant primary culture cell (Invitrogen, USA) treated with mitomycin C (Sigma, USA). First, the TT2F cell grown was treated with trypsin and suspended in HBS at 3×10⁷ cells/ml. Thereafter, 0.5 ml of the cell suspension was mixed with 10 μg of the vector DNA, loaded in a gene pulsar cuvette (distance between electrodes: 0.4 cm; Biorad, USA), and subjected to electroporation (capacity: 960 μF, voltage: 240 V, room temperature). After electroporation, the cells were suspended in 10 ml of ES medium (Shinichi Aizawa, as above) and seeded on a 100 mm plastic tissue-culture Petri dish (Falcon; Becton Dickinson, USA) having feeder cells seeded. After 36 hours, the medium was replaced with fresh ES medium containing 0.8 μg/ml puromycin (available from Sigma, USA). After 7 days, colonies generated. Of them, 72 colonies were picked up for each cell line, individually transferred to 24-well plates, and grown up to the confluent state. Two thirds of the grown cells were suspended in 0.2 ml of stock medium (ES medium+10% DMSO; Sigma, USA) and stored at −80° C. The remaining one thirds was seeded on a 12-well gelatin coated plate and cultured for 2 days. From 10⁶-10⁷ cells, genomic DNA was prepared by use of Puregene DNA Isolation Kits (Gentra System, USA). The genomic DNA of each puromycin-resistant ES cell line was digested with restriction enzyme EcoRI (Takara Shuzo, Japan) and separated by agarose gel electrophoresis. Subsequently, Southern blot was performed by use of, as a probe, the DNA fragment (XhoI-EcoRI, about 1.4 kb, FIG. 5), which was at the 3′ end of the Ig light chain Jκ-Cκ genomic DNA and used in the invention described in WO 00/10383 (see Example 48), thereby detecting homologous recombinants (HRs). The results are shown in Table 2.

TABLE 2

| Sequence present in RS region | | | | | | | |
|---|---|---|---|---|---|---|---|
| RS | Neo resistant | | | | Number of | Number of | |
| sequence | marker | SV40PE | SVE | Cell line | cells analyzed | HRs | % HR |
| x | ∘ | x | x | RS32 | 72 | 25 | 35% |
| x | x | x | x | RS32#15G- | 72 | 8 | 11% |
| x | x | ∘ | x | RSSV40PE8G-#36 | 72 | 34 | 47% |

The percentage of homologous recombinants was 11%, when pCκP2TPO(DT−) vector was used in the RS element targeting murine ES cell line (RS32#15G-) form which the neo resistant marker gene was removed; while 35% in the RS32 cell line having the Neo resistant marker gene. The homologous recombination efficiency of the cell line (RS32) having the neo resistant marker gene in the Cκ region is high, as in the cases of human EPO targeting vector (Example 13) and the pCκP2TPO vector (Example 27). It is further shown that the high homologous recombination efficiency disappeared after removal of the Neo resistant marker gene (RS32#15G-). More importantly, high homologous recombination efficiency (47%) was observed in the cell line (RSSV40PE8G-#36) from which the Neo resistant marker gene in the RS region was removed and in which the SV40PE sequence was remained. This demonstrates that the presence of SV40 enhancer/promoter (SV40PE) inserted into the RS element region improved the efficiency of homologous recombination in the Cc region that was at 25-kb upstream therefrom. In addition, in this Example, it was shown that the efficiency of homologous recombination of the targeting vector (pCκP2TPO(DT−)) with no negative selection marker (DT) was equivalent to that of the targeting vector (pCκP2TPO) with negative selection marker (DT).

These results suggest that the efficiency of homologous recombination in a target region can be enhanced by inserting the SV40 enhancer/promoter (SV40PE) sequence in a genomic region in the vicinity of the target region, even though SV40PE was not contained in the targeting vector. Furthermore, it was shown that the effect can not be achieved by enhancing the efficiency of negative selection but can be achieved by enhancing the homologous recombination efficiency itself

### Example 29

Obtaining ES Cell Having the Human FGF7 Gene Introduced by pCκP2FGF7 Vector

To obtain a murine ES cell line having the human FGF7-cDNA introduced downstream of the immunoglobulin κ light-chain gene by homologous recombination, the pCκP2FGF7 vector as prepared in Example 26 was introduced into each of the wild-type murine ES cells, namely TT2F-F8 cell line (Yagi et al., Analytical Biochem., 214:70, 1993), RS32 cell line (Example 4), RS32#15G- cell line (Example 12), RSSV40PE8G-#32 cell line (Example 17), RSSV40PE8G-#36 cell line (Example 17), RSSV40PE18G-#37 cell line (Example 17), RSSV40PE18G-#39 cell line (Example 17), RSSV4072bp37G-#4 cell line (Example 21), RSSV4072bp37G-#5 cell line (Example 21), RSSV4072bp38G-#26 cell line (Example 21), and RSSV4072bp38G-#28 cell line (Example 21) in accordance with the established method (Shinichi Aizawa, ibid). Culturing murine ES cells was performed in accordance with the method (Shinichi Aizawa, as above) using, as a trophocytes, the G418 resistant primary culture cell (Invitrogen, USA) treated with mitomycin C (Sigma, USA). First, the TT2F cell grown was treated with trypsin and suspended in HBS at 3×10⁷ cells/ml. Thereafter, 0.5 ml of the cell suspension was mixed with 10 μg of the vector DNA, loaded in a gene pulsar cuvette (distance between electrodes: 0.4 cm; Biorad, USA), and subjected to electroporation (capacity: 960 μF, voltage: 240 V, room temperature). After electroporation, the cells were suspended in 10 ml of ES medium (Shinichi Aizawa, as above) and seeded on a 100 mm plastic tissue-culture Petri dish (Falcon; Becton, Dickinson, USA) having feeder cells seeded. After 36 hours, the medium was replaced with a fresh ES medium containing 0.8 μg/ml puromycin (Sigma, USA). After 7 days, colonies generated. Colonies were picked up for each cell line, individually transferred to 24-well plates, and grown up to confluent state. Two thirds of the grown cells were suspended in 0.2 ml of stock medium (ES medium+10% DMSO; Sigma, USA) and stored at −80° C. The remaining one thirds was seeded on a 12-well gelatin coated plate and cultured for 2 days. From 10⁶-10⁷ cells, genomic DNA was prepared by use of Puregene DNA Isolation Kits (Gentra System, USA). The genomic DNA of each puromycin-resistant ES cell was digested with restriction enzyme EcoRI (Takara Shuzo, Japan) and separated by agarose gel electrophoresis. Subsequently, Southern blot was performed by use of, as a probe, the DNA fragment (XhoI-EcoRI, about 1.4 kb, FIG. 5), which was at the 3′ end of the Ig light chain Jκ-Cκ genomic DNA and used in the invention described in WO 00/10383 (see Example 48), thereby detecing homologous recombinants (HRs). The results are shown in Table 3.

TABLE 3

| Sequence present in RS region | | | | | | | |
|---|---|---|---|---|---|---|---|
| RS | Neo resistant | | | | Number of cells | Number of | |
| sequence | marker | SV40PE | SVE | Cell line | analyzed | HRs | % HR |
| ∘ | x | x | x | TT2-F8 | 72 | 8 | 11% |
| x | ∘ | x | x | RS32 | 24 | 16 | 67% |
| x | x | x | x | RS32#15G- | 72 | 9 | 13% |
| x | x | ∘ | x | RSSV40PE8G-#32 | 36 | 21 | 58% |
| x | x | ∘ | x | RSSV40PE8G-#36 | 33 | 25 | 76% |
| x | x | ∘ | x | RSSV40PE18G-#37 | 34 | 21 | 62% |
| x | x | ∘ | x | RSSV40PE18G-#39 | 36 | 13 | 36% |
| | | | | Sub total | 139 | 80 | 58% |
| x | x | x | ∘ | RSSV4072bp37G-#4 | 36 | 16 | 44% |
| x | x | x | ∘ | RSSV4072bp37G-#5 | 36 | 12 | 33% |
| x | x | x | ∘ | RSSV4072bp38G-#26 | 36 | 25 | 69% |
| x | x | x | ∘ | RSSV4072bp38G-#28 | 36 | 19 | 53% |
| | | | | Sub total | 144 | 72 | 50% |

The percentage of homologous recombinants was 13%, when pCκP2FGF7 vector was used in the RS element targeting murine ES cell line (RS32#15G-) form which the Neo resistant marker gene was removed, while 11% in the wild type TT2F-F8 cell line and 67% in the RS32 cell line carrying the Neo resistant marker gene. Thus, it was found that the homologous recombination efficiency of the cell line (RS32) having the neo resistant marker gene in the Cκ region is higher than that of the wild type ES cell line (TT2-F8), as in the case of the human EPO targeting vector (Example 13). It is further shown that the high homologous recombination efficiency disappeared after removal of the Neo resistant marker gene (RS32#15G-). Furthermore, in the cell lines RSSV40PE8G-#32, RSSV40PE8G-#36, RSSV40PE18G-#37, and RSSV40PE18G-#39, from which the Neo resistant marker gene in the RS region was removed and in which the SV40PE sequence were remained, high homologous recombination rate (58% in total) was observed. This demonstrates that the presence of SV40 enhancer/promoter (SV40PE) inserted into the RS element region enhanced the efficiency of homologous recombination in the Cκ region that was 25-kb upstream therefrom.

These results suggest that the efficiency of homologous recombination in a target region can be enhanced by inserting the SV40 enhancer/promoter (SV40PE) sequence in a genomic region in the vicinity of the target region, even though SV40PE was not contained in the targeting vector.

Furthermore, in the cell lines RSSV4072bp37G-#4, RSSV4072bp37G-#5, RSSV4072bp38G-#26, and RSSV4072bp38G-#28, from which the Neo resistant marker gene in the RS region was removed and in which the SV40 enhancer (tandem repeat of 72 bp-unit×2) sequence was remained, high homologous recombination rate (50% in total) was observed. This demonstrates that the presence of the SV40 enhancer (tandem repeat of 72 bp-unit×2) sequence inserted into the RS element region enhanced the efficiency of homologous recombination in the Cκ region that was at 25-kb upstream therefrom. These results suggest that the efficiency of homologous recombination in a target region can be enhanced by inserting the SV40 enhancer (tandem repeat of 72 bp-unit×2) sequence into a genomic region in the vicinity of the target region, even though the SV40 enhancer sequence was not contained in the targeting vector.

### INDUSTRIAL APPLICABILITY

According to the present invention, chimeric non-human animals (e.g., chimeric mouse) which express a desired protein can be obtained efficiently without fail compared to conventional methods. In the present invention, since an embryo devoid of the cells and/or tissue in which a gene encoding the desired protein to be introduced is expressed, is used as a host embryo, all of the cells and/or tissue in the chimeric non-human animal to be prepared are derived from the pluripotent cells containing the nucleic acid sequence or gene introduced. As a result, the desired protein can be expressed with high efficiency. Further in the present invention, the expression system of an immunoglobulin light chain, preferably κ chain, is used. The homologous recombination efficiency in the Igκ locus is 50 to 60% or more, when, as the embryonic stem cells, use is made of the murine ES cells in which a foreign enhancer is inserted, if necessary, together with a foreign gene under the transcriptional control, at a site within 100 kb or less, preferably 50 Kb or less, and more preferably, 30 Kb or less downstream of the 3′ end of the immunoglobulin κ chain constant region gene on chromosome, more specifically in the region where one of the alleles of the RS element is located. The homologous recombination efficiency achieved by the present invention is extremely high as compared to those of conventional methods. By virtue of this feature, the present invention is applicable to producing a desired protein by expressing a gene encoding the desired protein at a high level, or to analyzing the function of a gene or protein unknown in terms of in vivo function.

Sequence Listing Free Text

SEQ ID NOS: 1 to 18: synthetic oligonucleotide primer

SEQ ID NO: 19: SalI recognition sequence

SEQ ID NOS: 20 to 21: synthetic oligonucleotide primer

SEQ ID NO: 22: synthetic oligonucleotide primer comprising a multicloning site

SEQ ID NOS: 23 to 37: synthetic oligonucleotide primer

SEQ ID NO: 38: multicloning site

As to all publications, patents and patent applications cited in this specification, their disclosures are incorporated herein by reference in their entirety.

What is claimed is:

1. A pluripotent cell derived from a non-human animal, comprising a foreign enhancer at a site downstream of an immunoglobulin gene on chromosome.

2. The cell of claim 1, further comprising a desired foreign gene at the site downstream of the immunoglobulin gene and upstream of the foreign enhancer.

3. A chimeric non-human animal overexpressing a desired foreign gene, which is obtained by injecting a pluripotent cell of claim 1 or claim 2 into a host embryo of a non-human animal.

4. A non-human animal progeny overexpressing a desired foreign gene, which is produced by crossing chimeric non-human animals of claim 3.

5. A method of analyzing a function of a desired foreign gene, comprising comparing a phenotype based on a desired foreign gene which is overexpressed in a chimeric non-human animal of claim 3 or a non-human animal progeny of claim 4, with that of a control animal, and analyzing the function of the gene based on difference in phenotype.

6. A method of producing a useful protein by expressing a desired foreign gene in a chimeric non-human animal of claim 3 or a non-human animal progeny of claim 4, and recovering a produced protein, which is encoded by the gene expressed.


SEQUENCE LISTING (39 sequences)

SEQ ID NO: 1 - DNA, 28 bp, Artificial Sequence
  Description of Artificial Sequence Synthetic primer
tcgagtcgcgacaccggcgggcgcgccc

SEQ ID NO: 2 - DNA, 28 bp, Artificial Sequence
  Description of Artificial Sequence Synthetic primer
tcgagggcgcgcccgccggtgtcgcgac

SEQ ID NO: 3 - DNA, 29 bp, Artificial Sequence
  Description of Artificial Sequence Synthetic primer
ggccgcttaattaaggccggccgtcgacg

SEQ ID NO: 4 - DNA, 29 bp, Artificial Sequence
  Description of Artificial Sequence Synthetic primer
aattcgtcgacggccggccttaattaagc

SEQ ID NO: 5 - DNA, 49 bp, Artificial Sequence
  Description of Artificial Sequence Synthetic primer
ataagaatgcggccgcaaagctggtgggttaagactatctcgtgaagtg

SEQ ID NO: 6 - DNA, 45 bp, Artificial Sequence
  Description of Artificial Sequence Synthetic primer
acgcgtcgactcacaggttggtccctctctgtgtgtggttgctgt

SEQ ID NO: 7 - DNA, 42 bp, Artificial Sequence
  Description of Artificial Sequence Synthetic primer
ttggcgcgccctccctaggactgcagttgagctcagatttga

SEQ ID NO: 8 - DNA, 44 bp, Artificial Sequence
  Description of Artificial Sequence Synthetic primer
ccgctcgagtcttactgtctcagcaacaataatataaacagggg

SEQ ID NO: 9 - DNA, 25 bp, Artificial Sequence
  Description of Artificial Sequence Synthetic primer
catacaaacagatacacacatatac

SEQ ID NO: 10 - DNA, 25 bp, Artificial Sequence
  Description of Artificial Sequence Synthetic primer
gtcattaatggaaggaagctctcta

SEQ ID NO: 11 - DNA, 25 bp, Artificial Sequence
  Description of Artificial Sequence Synthetic primer
tcttactagagttctcactagctct

SEQ ID NO: 12 - DNA, 25 bp, Artificial Sequence
  Description of Artificial Sequence Synthetic primer
ggaaccaaagaatgaggaagctgtt

SEQ ID NO: 13 - DNA, 38 bp, Artificial Sequence
  Description of Artificial Sequence Synthetic primer
atctcgaggaaccactttcctgaggacacagtgatagg

SEQ ID NO: 14 - DNA, 38 bp, Artificial Sequence
  Description of Artificial Sequence Synthetic primer
atgaattcctaacactcattcctgttgaagctcttgac

SEQ ID NO: 15 - DNA, 32 bp, Artificial Sequence
  Description of Artificial Sequence Synthetic primer
atgaattcagacaaaggtcctgagacgccacc

SEQ ID NO: 16 - DNA, 42 bp, Artificial Sequence
  Description of Artificial Sequence Synthetic primer
atggatcctcgagtcgactggatttcagggcaactaaacatt

SEQ ID NO: 17 - DNA, 32 bp, Artificial Sequence
  Description of Artificial Sequence Synthetic primer
atgaattcgcccctctccctccccccccccta

SEQ ID NO: 18 - DNA, 38 bp, Artificial Sequence
  Description of Artificial Sequence Synthetic primer
atgaattcgtcgacttgtggcaagcttatcatcgtgtt

SEQ ID NO: 19 - DNA, 14 bp, Artificial Sequence
  Description of Artificial Sequence Synthetic Sal I recognition sequence
aatttgtcgactgc

SEQ ID NO: 20 - DNA, 44 bp, Artificial Sequence
  Description of Artificial Sequence Synthetic primer
cccaagctttggtgattattcagagtagttttagatgagtgcat

SEQ ID NO: 21 - DNA, 45 bp, Artificial Sequence
  Description of Artificial Sequence Synthetic primer
acgcgtcgactttgtctttgaactttggtccctagctaattacta

SEQ ID NO: 22 - DNA, 63 bp, Artificial Sequence
  Description of Artificial Sequence Synthetic primer with multicloning site
acgcgtcgacgcggccggccgcgctagcagacaaaggtcctgagacgccaccaccagctcccc

SEQ ID NO: 23 - DNA, 43 bp, Artificial Sequence
  Description of Artificial Sequence Synthetic primer
gaagatctcaagtgcaaagactcactttattgaatattttctg

SEQ ID NO: 24 - DNA, 42 bp, Artificial Sequence
  Description of Artificial Sequence Synthetic primer
ggaattcagacaaaggtcctgagacgccaccaccagctcccc

SEQ ID NO: 25 - DNA, 44 bp, Artificial Sequence
  Description of Artificial Sequence Synthetic primer
cccaagcttgcctcctcaaacctaccatggcccagagaaataag

SEQ ID NO: 26 - DNA, 51 bp, Artificial Sequence
  Description of Artificial Sequence Synthetic primer
ataagaatgcggccgcctcagagcaaatgggttctacaggcctaacaacct

SEQ ID NO: 27 - DNA, 44 bp, Artificial Sequence
  Description of Artificial Sequence Synthetic primer
ccggaattcctaacactcattcctgttgaagctcttgacaatgg

SEQ ID NO: 28 - DNA, 39 bp, Artificial Sequence
  Description of Artificial Sequence Synthetic primer
ccgctcgagcggccaccatgggggtgcacgaatgtcctg

SEQ ID NO: 29 - DNA, 33 bp, Artificial Sequence
  Description of Artificial Sequence Synthetic primer
ccgctcgagcggtcatctgtcccctgtcctgca

SEQ ID NO: 30 - DNA, 31 bp, Artificial Sequence
  Description of Artificial Sequence Synthetic primer
ggaattccgatcatattcaataacccttaat

SEQ ID NO: 31 - DNA, 31 bp, Artificial Sequence
  Description of Artificial Sequence Synthetic primer
actgccaagcccttaactttgttatcgtaag

SEQ ID NO: 32 - DNA, 28 bp, Artificial Sequence
  Description of Artificial Sequence Synthetic primer
ggcgcgccgctgtggaatgtgtgtcagt

SEQ ID NO: 33 - DNA, 28 bp, Artificial Sequence
  Description of Artificial Sequence Synthetic primer
ggcgcgccaagcttttgcaaaagcctag

SEQ ID NO: 34 - DNA, 28 bp, Artificial Sequence
  Description of Artificial Sequence Synthetic primer
ggcgcgccgtgtgtcagttagggtgtgg

SEQ ID NO: 35 - DNA, 28 bp, Artificial Sequence
  Description of Artificial Sequence Synthetic primer
ggcgcgccaggggcgggactatggttgc

SEQ ID NO: 36 - DNA, 39 bp, Artificial Sequence
  Description of Artificial Sequence Synthetic primer
acgcgtcgaccaccatgcacaaatggatactgacatgga

SEQ ID NO: 37 - DNA, 34 bp, Artificial Sequence
  Description of Artificial Sequence Synthetic primer
ctagctagcttaagttattgccataggaagaaag

SEQ ID NO: 38 - DNA, 24 bp, Artificial Sequence
  Description of Artificial Sequence Synthetic multicloning site
gtcgacgcggccggccgcgctagc

SEQ ID NO: 39 - DNA, 39 bp, Mus musculus
agtttctgcacgggcagtcagttagcagcactcactgtg